SEQUENCE LISTING (21 sequences)

SEQ ID NO: 1 - protein, 564 aa, Limosilactobacillus fermentum
MIYTPKWWWQNSVVYQVYPRSFQDSNHDGIGDLKGIISRLDYIKKLGADIIWLNPIYRSPNVDNGYDISDYRAIDPTFGSLTDFKELLTKAHELGLKIMMDLVVNHSSDENEWFKQSRQGKENPYRDYYIWRDPVDGHEPTNWGSYFSGSAWQYDETSGQYYLHLFAVKQPDLNWENEAVRHSVYDIMNWWADLGVDGFRMDVINLISKPAVYKDVPTAPGMQYGDVEPVVANGHRMHEFLQEMHQAVMAKHDLVTVGETPGATTDDAKKYANLEQTELNMVFEFEHVGLDGNDNPALGKWSDKKVSLPELRDNLVKWQTQLNGKAWNSLYWNNHDQPRVVSRFGNDDPKYRVVSAKMLATMLHCLQGTPYIYAGEELGMTNTTFNSLSDYRDLESINAYHQLVDEEHLVDGKTMSRYLAIHSRDNARTPMQWDDSKNAGFSDAEPWIAVNPNYSEINAKAALADPSSVFYHYQKLIQMRHDLPVMTEGKFALVNGNELDEQVFAYTRDDGETTLLVVANFTKETIKREYAAGQGKLLLSNYEDDMGETLRPYEAKVYEFSSKR

SEQ ID NO: 2 - DNA, 1695 bp, Limosilactobacillus fermentum
  source 1..1695: mol_type = genomic DNA
atgatatatacaccaaaatggtggtggcaaaattcagttgtctaccaagtctatccacggagttttcaagacagcaatcatgatggcattggtgatttaaaaggaatcatcagtcggcttgactatattaaaaaactaggtgctgatattatctggctcaatccaatctaccgttcgccaaacgttgataatgggtatgacatcagtgattatcgggcaattgatccaacttttggctcattgaccgactttaaggagctgctgacaaaggctcatgaacttggattaaaaataatgatggatctggtggtcaatcactcatcagatgaaaatgaatggtttaagcaaagccgccaaggaaaagaaaatccataccgtgactattatatctggcgtgatccagtcgatggccatgaaccaactaattggggatcatacttctctggttcagcttggcagtacgatgagacaagcggtcaatactaccttcacctgttcgctgtaaagcagccggatctaaactgggaaaatgaagccgttcgtcattcggtatacgacatcatgaattggtgggcagatctcggtgtcgatggatttcgaatggatgtcatcaacttaatttcaaaaccggctgtctacaaagacgttccaaccgcgcccggaatgcagtatggcgatgtcgagccggttgttgccaatggtcatcggatgcacgaattccttcaagaaatgcatcaagctgtaatggccaagcatgatttggtaacagttggcgaaacacctggtgctacaacagatgatgccaagaagtacgccaaccttgaacagactgagctgaacatggtttttgagtttgagcatgtcggcttagacggtaacgacaacccagctttaggaaaatggagcgacaaaaaagtcagtctgccagaattgcgagataacctggtgaaatggcagactcagctgaatggtaaggcttggaactcgctttactggaacaaccatgatcagccacgcgtcgtatcccgttttggcaatgatgatccaaaatatcgtgtcgtttcggctaaaatgttggcaacaatgctgcactgtctgcaaggcacgccttatatctacgcaggtgaggaactgggaatgaccaacacgacctttaactctttgtctgactatcgtgatttggagagcattaatgcctaccatcagctggttgacgaagagcatctagttgatggaaagacaatgagcagatatctggcaattcattctcgggacaatgccagaacgccaatgcaatgggatgacagcaagaatgccggattttcggatgctgaaccttggatcgcggtcaatcccaactattcagagatcaacgcaaaggcggcactggcggatccatcatccgtcttctatcactaccagaaactcattcaaatgcgccatgacttgccagtaatgacagaaggaaagtttgcgttggtcaatggcaacgaattggatgagcaggtctttgcttacacgcgtgatgatggagaaacaacgctgcttgtagtagccaactttactaaggaaacaatcaagcgagaatacgcggctggtcaaggcaagctcttattaagcaactacgaggatgacatgggagaaactctgcgtccatatgaagctaaagtatatgaattcagttcaaagaggtaa

SEQ ID NO: 3 - protein, 564 aa, synthetic construct
MIYTPKWWWQNSVVYQVYPRSFQDSNHDGIGDLKGIISRLDYIKKLGADIIWLNPIYRSPNVDNGYDISDYRAIDPTFGSLTDFKELLTKAHELGLKIMMDLVVNHSSDENEWFKQSRQGKENPYRDYYIWKDPVDGHEPTNWGSYFSGSAWQYDETSGQYYLHLFAVKQPDLNWENEAVRHSVYDIMNWWADLGVDGFRMDVINLISKPAVYKDVPTAPGMQYGDVEPVVANGHRMHEFLQEMHQAVMAKHDLVTVGETPGATTDDAKKYANLEQTELNMVFEFEHVGLDGNDNPALGKWSDKKVSLPELRDNLVKWQTQLNGKAWNSLYWNNHDQPRVVSRFGNDDPKYRVVSAKMLATMLHCLQGTPYIYAGEELGMTNTTFNSLSDYRDLESINAYHQLVDEEHLVDGKTMSRYLAIHSRDNARTPMQWDDSKNAGFSDAEPWIAVNPNYSEINAKAALADPSSVFYHYQKLIQMRHDLPVMTEGKFALVNGNDLDEQVFAYTRDDGETTLLVVANFTKETIKREYDAGQGKLLLSNYEDDMGETLRPYEAKVYEFSSKR

SEQ ID NO: 4 - protein, 564 aa, synthetic construct
MIYTPKWWWQNSVVYQVYPRSFQDSNHDGIGDLKGIISRLDYIKKLGADIIWLNPIYRSPNVDNGYDISDYRAIDPTFGSLTDFKELLTKAHELGLKIMMDLVVNHSSDENEWFIQSRQGKENPYRDYYIWKDPVDGHEPTNWGSYFSGSAWQYDETSGQYYLHLFAVKQPDLNWENEAVRHSVYDIMNWWADLGVDGFRMDVINLISKPAVYKDVPTAPGMQYGDVEPVVANGHRMHEFLQEMHQAVMAKHDLVTVGETPGATTDDAKKYANLEQTELNMVFEFEHVGLDGNDNPALGKWSDKKVSLPELRDNLVKWQTQLNGKAWNSLYWNNHDQPRVVSRFGNDDPKYRVVSAKMLATMLHCLQGTPYIYAGEELGMTNTTFNSLSDYRDLESINAYHQLVDEEHLVDGKTMSRYLAIHSRDNARTPMQWDDSKNAGFSDGEPWIAVNPNYSEINAKAALADPSSVFYHYQKLIQMRHDLPVMTEGKFALVNGNELDEQVFAYTRDDGETTLLVVANFTKETIKREYDAGQGKLLLSNYEDDMGETLRPYEAKVYEFSSKR

SEQ ID NO: 5 - protein, 564 aa, synthetic construct
MIYTPKWWWQNSVVYQVYPRSFQDSNHDGIGDLKGIISRLDYIKKLGADIIWLNPIYRSPNVDNGYDISDYRAIDPTFGSLTDFKELLTKAHELGLKIMMDLVVNHSSDENEWFIQSRQGKENPYRDYYIWKDPVDGHEPTNWGSYFSGSAWQYDETSGQYYLHLFAVKQPDLNWENEAVRHSVYDIMNWWADLGVDGFRMDVINLISKPAVYKDVPTAPGMQYGSVEPVVANGHRMHEFLQEMHQAVMAKHDLVTVGETPGATTDDAKKYANLEQTELNMVFEFEHVGLDGNDNPALGKWSDKKVSLPELRDNLVKWQTQLNGKAWNSLYWNNHDQPRVVSRFGNDDPKYRVVSAKMLATMLHCLQGTPYIYAGEELGMTNTTFNSLSDYRDLESINAYHQLVDEEHLVDGKTMSRYLAAHSRDNARTPMQWDDSKNAGFSDGEPWIAVNPNYSEINAKAALADPSSVFYHYQKLIQMRHDLPVMTEGKFALVNGNELDEQVFAYTRDDGETTLLVVANFTKETIKREYDAGQGKLLLSNYEDDMGETLRPYEAKVYELSSKR

SEQ ID NO: 6 - protein, 564 aa, synthetic construct
MIYTPKWWWQNSVVYQVYPRSFQDSNHDGIGDLKGIISRLDYIKKLGADIIWLNPIYRSPNVDNGYDISDYRAIDPTFGSLADFKELLTKAHELGLKIMMDLVVNHSSDENEWFIQSRQGKENPYRDYYIWKDPVDGHEPTNWGSYFSGSAWQYDETSGQYYLHLFAVKQPDLNWENEAVRHSVYDIMNWWADLGVDGFRMDVINLISKPAVYKDVPTAPGMQYGSVEPVVANGHRMHEFLQEMHQAVMAKHDLVTVGETPGATTDDAKKYANLEQTELNMVFEFEHVGLDGNDNPALGKWSDKKVSLPELRDNLVKWQTQLNGKAWNSLYWNNHDQPRVVSRFGNDDPKYRVVSAKMLATMLHCLQGTPYIYAGEELGMTNTTFNSLSDYRDIESINAYHQLVDEEHLVDGKTMSRYLAIHSRDNARTPMQWDDSKNAGFSDGEPWIAVNPNYSEINAKAALADPSSVFYHYQKLIQMRHDLPVMTEGKFALVNGNELDEQVFAYTRDDGETTLLVVANFTKETIKREYDAGQGKLLLSNYEDDMGETLRPYEAKVYEFSSKR

SEQ ID NO: 7 - protein, 564 aa, synthetic construct
MIYTPKWWWQNSVVYQVYPRSFQDSNHDGIGDLKGIISRLDYIKKLGADIIWLNPIYRSPNVDNGYDISDYRAIDPTFGSLTDFKELLTKAHELGLKIMMDLVVNHSSDENEWFIQSRQGKENPYRDYYIWKDPVDGHEPTNWGSYFSGSAWQYDETSGQYYLHLFAVKQPDLNWENEAVRHSVYDIMNWWADLGVDGFRMDVINLISKPAVYKDVPTAPGMQYGSVEPVVANGHRMHEFLQEMHQAVMAKHDLVTVGETPGATTDDAKKYANLEQTELNMVFEFEHVGLDGNDNPALGKWSDKKVSLPALRDNLVKWQTQLNGKAWNSLYWNNHDQPRVVSRFGNDDPKYRVVSAKMLATMLHCMQGTPYIYAGEELGMTNTTFNSLSDYRDLESINAYHQLVDEEHLVDGKTMSRYLAAHSRDNARTPMQWDDSKNAGFSDGEPWIAVNPNYSEINAKAALADPSSVFYHYQKLIQMRHDLPVMTEGKFALVNGNELDEQVFAYTRDDGETTLLVVANFTKETIKREYDAGQGKLLLSNYEDDMGETLRPYEAKVYEVSSKR

SEQ ID NO: 8 - protein, 564 aa, synthetic construct
MIYTPKWWWQNSVVYQVYPRSFQDSNHDGIGDLKGIISRLDYIKKLGADIIWLNPIYRSPNVDNGYDISDYRAIDPTFGSLTDFKELLTKAHELGLKIMMDLVVNHSSDENEWFKQSRQGKENPYRDYYIWRDPVDGHEPTNWGSYFSGSAWQYDEASGQYYLHLFAVKQPDLNWENEAVRHSVYDIMNWWADLGVDGFRMDVINLISKPAVYKDVPTAPGMQYGDVEPVVANGHRMHEFLQEMHQAVMAKHDLVTVGETPGATTDDAKKYANLDQTELNMVFEFEHVGLDGNDNPALGKWSDKKVSLPELRDNLVKWQTQLNGKAWNSLYWNNHDQPRVVSRFGNDDPKYRVVSAKMLATMLHCMQGTPYIYAGEELGMTNTTFNSLSDYRDLESINAYHQLVDEEHLVDGKTMSRYLAIHSRDNARTPMQWDDSKNAGFSDAEPWIAVNPNYSEINAKAALADPSSVFYHYQKLIQMRHDLPVMTEGKFALVNGNELDEQVFAYTRDDGETTLLVVANFTKETIKREYAAGQGKLLLSNYEDDMGETLRPYEAKVYEFSSKR

SEQ ID NO: 9 - protein, 564 aa, synthetic construct
MIYTPKWWWQNSVVYQVYPRSFQDSNHDGIGDLKGIISRLDYIKKLGADIIWLNPIYRSPNVDNGYDISDYRAIDPTFGSLTDFKELLTKAHELGLKIMMDLVVNHSSDENEWFKQSRQGKENPYRDYYIWRDPVDGHEPTNWGSYFSGSAWQYDETSGQYYLHLFAVKQPDLNWENEAVRHSVYDIMNWWADLGVDGFRMDVINLISKPAVYKDVPTAPGMQYGDVEPVVANGHRMHEFLQEMHQAVMAKHDLVTVGETPGATTDDAKKYANLEQTELNMVFEFEHVGLDGNDNPALGKWSDKKVSLPELRDNLVKWQTQLNGKAWNSLYWNNHDQPRVVSRFGNDDPKYRVVSAKMLATMLHCLQGTPYIYAGEELGMTNTTFNSLSDYRDLESINAYHQLVDEEHLVDGKTMSRYLAIHSRDNARTPMQWDDSKNAGFSDAEPWIAVNPNYSEINAKAALADPSSVFYHYQKLIQMRHDLPVMTEGKFALVNGNELDEQVFAYTRDDGETTLLVVANFTKETIKREYAAEQGKLLLSNYDDDMSETLRPYEAKVYEFSSKR

SEQ ID NO: 10 - protein, 564 aa, synthetic construct
MIYTPKWWWQNSVVYQVYPRSFQDSNHDGIGDLKGIISRLDYIKKLGADIIWLNPIYRSPNVDNGYDISDYRAIDPTFGSLTDFKELLAKAHELGLKIMMDLVVNHSSDENEWFKQSRQGKENPYRDYYIWKDPVDGHEPTNWGSYFSGSAWQYDETSGQYYLHLFAVKQPDLNWENPAVRHSVYDIMNWWADLGVDGFRMDVINLISKPAVYKDVPTAPGMQYGDVEPVVANGHRMHEFLQEMHQAVMAKHDLVTVGETPGATTDDAKKYANLEQTELNMVFEFEHVGLDGNDNPALGKWSDKKVSLPELRDNLVKWQTQLNGKAWNSLYWNNHDQPRVVSRFGNDDPKYRVVSAKMLATMLHCLQGTPYIYAGEELGMTNTTFNSLSDYRDLESINAYHQLVDEEHLVDGKTMSRYLAIHSRDNARTPMQWDDSKNAGFSDAEPWIAVNPNYSEINAKAALADPSSVFYHYQKLIQMRHDLPVMTEGKFALVNGNDLDEQVFAYTRDDGETTLLVVANFTKETIKREYDAGQGKLLLSNYEDDMGETLRPYEAKVYELSSKR

SEQ ID NO: 11 - protein, 564 aa, synthetic construct
MIYTPKWWWQNSVVYQVYPRSFQDSNHDGIGDLKGIISRLDYIKKLGADIIWLNPIYRSPNVDNGYDISDYRAIDPTFGSLTDFKELLTKAHELGLKIMMDLVVNHSSDENEWFIQSRQGKENPYRDYYIWKDPVDGHEPTNWGSYFSGSAWQYDETSGQYYLHLFAVKQPDLNWENEAVRHSVYEIMNWWADLGVDGFRMDVINLISKPAVYKDVPTAPGMQYGDVEPVVANGHRMHEFLQEMHQAVMAKHDLVTVGETPGATTDDAKKYANLEQTELNMVFEFEHVGLDGNDNPALGKWSDKKVSLPELRDNLVKWQTQLNGKAWNSLYWNNHDQPRVVSRFGNDDPKYRVVSAKMLATMLHCLQGTPYIYAGEELGMTNTTFNSLSDYRDLESINAYHQLVDEEHLVDGKTMSRYLAIHSRDNARTPMQWDDSKNAGFSDAEPWIAVNPNYAEINAKAALADPSSVFYHYQKLIQMRHDLPVMTEGKFALVNGNDLDEQVFAYTRDDGETTLLVVANFTKETIKREYDAGQGKLLLSNYEDDMGETLRPYEAKVYEFSSKR

SEQ ID NO: 12 - protein, 564 aa, synthetic construct
MIYTPKWWWQNSVVYQVYPRSFQDSNHDGIGDLKGIISRLDYIKKLGADIIWLNPIYRSPNVDNGYDISDYRAIDPTFGSLADFKELLTKAHELGLKIMMDLVVNHSSDENEWFKQSRQGKENPYRDYYIWKDPVDGHEPTNWGSYFSGSAWQYDETSGQYYLHLFAVKQPDLNWENPAVRHSVYDIMNWWADLGVDGFRMDVINLISKPAVYKDVPTAPGMQYGDVEPVVANGHRMHEFLQEMHQAVMAKHDLVTVGETPGATTDDAKKYANLEQTELNMVFEFEHVGLDGNDNPALGKWSDKKVSLPELRDNLVKWQTQLNGKAWNSLYWNNHDQPRVVSRFGNDDPKYRVVSAKMLATMLHCLQGTPYIYAGEELGMTNTTFNSLSDYRDLESINAYHQLVDEEHLVDGKTMSRYLAIHSRDNARTPMQWDDSKNAGFSDAEPWIAVNPNYSEINAKAALADPSSVFYHYQKLIQMRHDLPVMTEGKFALVNGNDLDDQVFAYTRDDGETTLLVVANFTKETIKREYDAGQGKLLLSNYEDDMGETLRPYEAKVYEFSSKR

SEQ ID NO: 13 - protein, 532 aa, Saccharomyces cerevisiae
MLLQAFLFLLAGFAAKISASMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 14 - protein, 513 aa, Saccharomyces cerevisiae
SMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 15 - DNA, 1599 bp, Saccharomyces cerevisiae
  source 1..1599: mol_type = genomic DNA
atgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactcattgggaagatgaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgctaatgaaggtttcttaggctaccaatatgaatgtccaggtttgattgaagtcccaactgagcaagatccttccaaatcctattgggtcatgtttatttctatcaatccaggtgcacctgctggcggttccttcaaccaatattttgttggatccttcaatggtactcattttgaagcgtttgacaatcaatctagagtggtagattttggtaaggactactatgccttgcaaactttcttcaacacagacccaacgtacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttcccgacttatcactttggttcaagggtttagaagatcctgaagaatatttaagaatgggttttgaagccagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatag

SEQ ID NO: 16 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDEKDAKWHLYFQYNPNDTVWGTPLFWGHATSDDLTHWEDEPIAIAPKRNDSGAYSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISAAGPWSRFATNTTLTKANSYNVDLSNSTGTLEFELVYAVNTTQTISKSVFADLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 17 - protein, 513 aa, synthetic construct
SMTNETSDRPLVHFTPNKGWMNDPNGLWYDEKDAKWHLYFQYNPNDTVWGTPLFWGHATSDDLTHWEDEPIAIAPKRNDSGAYSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISAAGPWSRFATNTTLTKANSYNVDLSNSTGTLEFELVYAVNTTQTISKSVFADLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 18 - protein, 89 aa, unidentified
  source 1..89: note = FAKS signal sequence
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 19 - protein, 532 aa, synthetic construct
MLLQAFLFLLAGFAAKISASMTNETSDRPLVHFTPNKGWMNDPNGLWYDEKDAKWHLYFQYNPNDTVWGTPLFWGHATSDDLTNWEDQPIAIAPKRNDSGAYSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISAAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 20 - protein, 513 aa, synthetic construct
SMTNETSDRPLVHFTPNKGWMNDPNGLWYDEKDAKWHLYFQYNPNDTVWGTPLFWGHATSDDLTNWEDQPIAIAPKRNDSGAYSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISAAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 21 - protein, 6 aa, synthetic construct
HHHHHH

(19) United States
(12) Patent Application Publication
(10) Pub. No.: US 2025/0243475 A1
(43) Pub. Date: Jul. 31, 2025

(54) METHODS AND COMPOSITIONS FOR TREATING CONGENITAL SUCRASE-ISOMALTASE DEFICIENCY

(71) Applicant: Anagram Therapeutics, Inc., Framingham, MA (US)

(72) Inventors: Sridhar Govindarajan, Los Altos, CA (US); Mark Welch, Fremont, CA (US); Alexey Margolin, Newton, MA (US)

(21) Appl. No.: 18/992,838

(22) PCT Filed: Jul. 13, 2023
(86) PCT No.: PCT/US2023/070180

§ 371 Date: Jan. 9, 2025

Related U.S. Application Data
(60) Provisional application No. 63/388,810, filed on Jul. 13, 2022.
(60) Provisional application No. 63/388,845, filed on Jul. 13, 2022.

(51) Int. Cl. C12N 9/44; A61K 38/00; C12N 1/18; C12N 1/20; C12N 9/26; C12N 15/70; C12N 15/81

(52) U.S. Cl. CPC C12N 9/2451; C12N 1/18; C12N 1/20; C12N 9/2431; C12N 15/70; C12N 15/81; A61K 38/00; C12Y 302/0101; C12Y 302/01026

(57) ABSTRACT

Disclosed are recombinant mutant isomaltase enzymes, and pharmaceutical compositions comprising an isomaltase enzyme, e.g., a recombinant mutant isomaltase enzyme, and an optional invertase enzyme. The enzymes and compositions can be used, among other things, to treat congenital sucrase-isomaltase deficiency (CSID).

### CROSS-REFERENCE TO RELATED APPLICATIONS

This application claims the benefit of and priority to U.S. Provisional Patent Application No. 63/388,845, filed on Jul. 13, 2022 and U.S. Provisional Patent Application No. 63/388,810, filed on Jul. 13, 2022, the disclosures of each of which are hereby incorporated by reference in their entireties for all purposes.

### REFERENCE TO A SEQUENCE LISTING XML

This application contains a Sequence Listing which has been submitted electronically in XML format. The Sequence Listing XML is incorporated herein by reference. Said XML file, created on Jul. 12, 2023, is named ANR-005WO_SL.xml and is 32,102 bytes in size.

### FIELD OF THE INVENTION

The invention relates generally to recombinant mutant isomaltase enzymes and methods and compositions for treating congenital sucrase-isomaltase deficiency (CSID).

### BACKGROUND

Congenital sucrose-isomaltase deficiency (CSID) is a rare autosomal recessive inherited disease of the small intestine resulting from genetic mutations in sucrase-isomaltase, an enzyme complex responsible for catalyzing the hydrolysis of dietary sucrose and starch (Ritz et al. GASTROENTEROLOGY 125(6):1678-85). Decreased or absent sucrase and/or isomaltase enzymatic activity has been found in patients with CSID, and investigations at the subcellular and molecular levels in intestinal biopsy specimens have led to the description of several phenotypes, differing in transport efficiency, processing, and sorting of the protein, which result in impaired physiologic functions (Ritz et al. (2003) supra, Jacob et al. (2000) J. CLIN. INVEST. 106(2):281-7, Alfalah et al. (2009) GASTROENTEROLOGY 136(3):883-92, and Ouwendijk (1996) J. CLIN. INVEST. 97(3):633-41).

Affected subjects may present with osmotic diarrhea, mild steatorrhea, chronic diarrhea, irritability, and vomiting after consuming sucrose. The difficulty in providing adequate nutrition to these subjects can lead to dehydration, metabolic acidosis, hypercalcemia, failure to thrive, and developmental delay (Cohen (2016) MOL. CELL. PEDIATR. 3:5). Failure to absorb dietary disaccharides and starch has implications for the absorption of other nutrients and the hormonal regulation of gastrointestinal function.

The prevalence of CSID in the European population has been estimated at 1 in 5000, but it is higher among the indigenous populations of Alaska, Greenland, and Canada (Marcadier et al. (2015) CANADIAN MED. ASSOC. J. 187(2):102-107). There is also evidence that heterozygous carriers also experience symptoms of CSID, such as chronic diarrhea, abdominal pain, and bloating. It is currently estimated that 2-9% of Americans of European descent may be affected, suggesting that sucrase-isomaltase deficiency has been underrecognized (DeJonge et al. (2014) GASTROENTEROLOGY 146: S705).

CSID currently may be treated by the oral administration of a solution of sacrosidase (SUCRAID®). However, sacrosidase does not fully replace the enzymatic activity of the endogenous sucrase-isomaltase enzyme complex. The liquid formulation of sacrosidase in 50% glycerol is thermally unstable and unstable under acidic conditions. As a result, sacrosidase solution must be discarded four weeks after opening and cannot be used in conjunction with warm beverages and infant formula or fruit juices, negatively impacting ease of use and patient compliance.

Although developments have been made to date, there is still an ongoing need for new and effective pharmaceutical compositions and therapies for treating and managing CSID.

### SUMMARY

The invention is based, in part, upon the development of recombinant mutant isomaltase (α-glucosidase) enzymes (also referred to as isomaltase muteins) that are active in humans and have greater stability and/or activity than naturally occurring enzymes. In particular, the recombinant mutant enzymes disclosed herein may exhibit improved pH stability, thermal stability, and/or stability against proteolytic digestion compared to naturally occurring versions of the enzyme. Furthermore, the recombinant mutant enzymes disclosed herein can have greater specific activity than a wild type isomaltase (α-glucosidase) enzyme. Furthermore, it is contemplated that the recombinant mutant enzymes disclosed herein, given their enhanced stability, may be suitable for oral administration, and potentially safer, more tolerable, and/or more effective than commercially available isomaltase (α-glucosidase) enzymes. The invention is also based, in part, upon the development of pharmaceutical compositions, e.g., solid pharmaceutical compositions, comprising an isomaltase (α-glucosidase) enzyme and an optional sucrase (invertase) enzyme that are stable at room temperature, allowing for easier distribution, storage, and administration to subjects relative to compositions that are unstable at room temperature and/or require refrigeration. The recombinant enzymes and compositions can be used, among other things, to treat congenital sucrase-isomaltase deficiency (CSID).

Accordingly, in one aspect, the disclosure relates to a recombinant mutant isomaltase enzyme (e.g., a recombinant mutant Gram positive bacterial isomaltase enzyme, e.g., a recombinant mutant Limosilactobacillus fermentum isomaltase enzyme, formerly Lactobacillus fermentum). In certain embodiments, the isomaltase has (i) increased activity at acidic pH (e.g., in the range of about pH 4.0 to about 6.0) relative to a corresponding wild-type isomaltase, (ii) increased activity at neutral pH (e.g., about pH 7.0) relative to a corresponding wild-type isomaltase, (iii) increased stability at acidic pH (e.g., about pH 4) relative to a corresponding wild-type isomaltase, (iv) increased thermal stability relative to a corresponding wild-type isomaltase, (v) increased proteolytic stability in the presence of pancreatin and/or pepsin relative to a corresponding wild-type isomaltase, or (vi) a combination of any of the foregoing features (i) to (v).

In certain embodiments, the isomaltase comprises: a substitution of a residue at a position corresponding to position 115 of wild-type L. fermentum isomaltase; a substitution of a residue at a position corresponding to position 132 of wild-type L. fermentum isomaltase; a substitution of a residue at a position corresponding to position 226 of wild-type L. fermentum isomaltase; a substitution of a residue at a position corresponding to position 310 of wild-type L. fermentum isomaltase; a substitution of a residue at a position corresponding to position 366 of wild-type L. fermentum isomaltase; a substitution of a residue at a position corresponding to position 421 of wild-type L. fermentum isomaltase; a substitution of a residue at a position corresponding to position 444 of wild-type L. fermentum isomaltase; a substitution of a residue at a position corresponding to position 531 of wild-type L. fermentum isomaltase; a substitution of a residue at a position corresponding to position 560 of wild-type L. fermentum isomaltase; a substitution of a residue at a position corresponding to position 12 of wild-type L. fermentum isomaltase; a substitution of a residue at a position corresponding to position 82 of wild-type L. fermentum isomaltase; a substitution of a residue at a position corresponding to position 86 of wild-type L. fermentum isomaltase; a substitution of a residue at a position corresponding to position 89 of wild-type L. fermentum isomaltase; a substitution of a residue at a position corresponding to position 93 of wild-type L. fermentum isomaltase; a substitution of a residue at a position corresponding to position 122 of wild-type L. fermentum isomaltase; a substitution of a residue at a position corresponding to position 136 of wild-type L. fermentum isomaltase; a substitution of a residue at a position corresponding to position 157 of wild-type L. fermentum isomaltase; a substitution of a residue at a position corresponding to position 168 of wild-type L. fermentum isomaltase; a substitution of a residue at a position corresponding to position 178 of wild-type L. fermentum isomaltase; a substitution of a residue at a position corresponding to position 182 of wild-type L. fermentum isomaltase; a substitution of a residue at a position corresponding to position 186 of wild-type L. fermentum isomaltase; a substitution of a residue at a position corresponding to position 211 of wild-type L. fermentum isomaltase; a substitution of a residue at a position corresponding to position 246 of wild-type L. fermentum isomaltase; a substitution of a residue at a position corresponding to position 269 of wild-type L. fermentum isomaltase; a substitution of a residue at a position corresponding to position 275 of wild-type L. fermentum isomaltase; a substitution of a residue at a position corresponding to position 309 of wild-type L. fermentum isomaltase; a substitution of a residue at a position corresponding to position 394 of wild-type L. fermentum isomaltase; a substitution of a residue at a position corresponding to position 455 of wild-type L. fermentum isomaltase; a substitution of a residue at a position corresponding to position 486 of wild-type L. fermentum isomaltase; a substitution of a residue at a position corresponding to position 498 of wild-type L. fermentum isomaltase; a substitution of a residue at a position corresponding to position 501 of wild-type L. fermentum isomaltase; a substitution of a residue at a position corresponding to position 502 of wild-type L. fermentum isomaltase; a substitution of a residue at a position corresponding to position 514 of wild-type L. fermentum isomaltase; a substitution of a residue at a position corresponding to position 524 of wild-type L. fermentum isomaltase; a substitution of a residue at a position corresponding to position 533 of wild-type L. fermentum isomaltase; a substitution of a residue at a position corresponding to position 535 of wild-type L. fermentum isomaltase; a substitution of a residue at a position corresponding to position 543 of wild-type L. fermentum isomaltase; a substitution of a residue at a position corresponding to position 554 of wild-type L. fermentum isomaltase; a substitution of a residue at a position corresponding to position 556 of wild-type L. fermentum isomaltase; or a combination of any of the foregoing substitutions, wherein the wild-type L. fermentum isomaltase comprises the amino acid sequence of SEQ ID NO:1. Also contemplated herein are isomaltase enzymes comprising the foregoing substitutions at the corresponding positions in SEQ ID NO: 1, namely wild-type isomaltase enzyme derived from L. fermentum (see TABLE 1 below).

In certain embodiments, in the isomaltase, the residue at a position corresponding to position 115 of wild-type L. fermentum isomaltase is substituted by I; the residue at a position corresponding to position 132 of wild-type L. fermentum isomaltase is substituted by K; the residue at a position corresponding to position 226 of wild-type L. fermentum isomaltase is substituted by S; the residue at a position corresponding to position 310 of wild-type L. fermentum isomaltase is substituted by A; the residue at a position corresponding to position 366 of wild-type L. fermentum isomaltase is substituted by M; the residue at a position corresponding to position 421 of wild-type L. fermentum isomaltase is substituted by A; the residue at a position corresponding to position 444 of wild-type L. fermentum isomaltase is substituted by G; the residue at a position corresponding to position 531 of wild-type L. fermentum isomaltase is substituted by D; the residue at a position corresponding to position 560 of wild-type L. fermentum isomaltase is substituted by V; the residue at a position corresponding to position 12 of wild-type L. fermentum isomaltase is substituted by A; the residue at a position corresponding to position 82 of wild-type L. fermentum isomaltase is substituted by A; the residue at a position corresponding to position 86 of wild-type L. fermentum isomaltase is substituted by Q; the residue at a position corresponding to position 89 of wild-type L. fermentum isomaltase is substituted by A; the residue at a position corresponding to position 93 of wild-type L. fermentum isomaltase is substituted by K; the residue at a position corresponding to position 122 of wild-type L. fermentum isomaltase is substituted by D; the residue at a position corresponding to position 136 of wild-type L. fermentum isomaltase is substituted by N; the residue at a position corresponding to position 157 of wild-type L. fermentum isomaltase is substituted by A; the residue at a position corresponding to position 168 of wild-type L. fermentum isomaltase is substituted by K; the residue at a position corresponding to position 178 of wild-type L. fermentum isomaltase is substituted by D; the residue at a position corresponding to position 178 of wild-type L. fermentum isomaltase is substituted by P; the residue at a position corresponding to position 182 of wild-type L. fermentum isomaltase is substituted by R; the residue at a position corresponding to position 186 of wild-type L. fermentum isomaltase is substituted by E; the residue at a position corresponding to position 211 of wild-type L. fermentum isomaltase is substituted by E; the residue at a position corresponding to position 246 of wild-type L. fermentum isomaltase is substituted by R; the residue at a position corresponding to position 269 of wild-type L. fermentum isomaltase is substituted by R; the residue at a position corresponding to position 275 of wild-type L. fermentum isomaltase is substituted by D; the residue at a position corresponding to position 309 of wild-type L. fermentum isomaltase is substituted by R; the residue at a position corresponding to position 394 of wild-type L. fermentum isomaltase is substituted by I; the residue at a position corresponding to position 455 of wild-type L. fermentum isomaltase is substituted by A; the residue at a position corresponding to position 486 of wild-type L. fermentum isomaltase is substituted by I; the residue at a position corresponding to position 498 of wild-type L. fermentum isomaltase is substituted by N; the residue at a position corresponding to position 498 of wild-type L. fermentum isomaltase is substituted by D; the residue at a position corresponding to position 501 of wild-type L. fermentum isomaltase is substituted by D; the residue at a position corresponding to position 502 of wild-type L. fermentum isomaltase is substituted by E; the residue at a position corresponding to position 514 of wild-type L. fermentum isomaltase is substituted by K; the residue at a position corresponding to position 524 of wild-type L. fermentum isomaltase is substituted by Q; the residue at a position corresponding to position 533 of wild-type L. fermentum isomaltase is substituted by E; the residue at a position corresponding to position 535 of wild-type L. fermentum isomaltase is substituted by S; the residue at a position corresponding to position 543 of wild-type L. fermentum isomaltase is substituted by D; the residue at a position corresponding to position 543 of wild-type L. fermentum isomaltase is substituted by N; the residue at a position corresponding to position 547 of wild-type L. fermentum isomaltase is substituted by S; the residue at a position corresponding to position 551 of wild-type L. fermentum isomaltase is substituted by N; the residue at a position corresponding to position 554 of wild-type L. fermentum isomaltase is substituted by Q; the residue at a position corresponding to position 556 of wild-type L. fermentum isomaltase is substituted by I; the residue at a position corresponding to position 560 of wild-type L. fermentum isomaltase is substituted by I; the residue at a position corresponding to position 560 of wild-type L. fermentum isomaltase is substituted by L; or the isomaltase comprises a combination of any of the foregoing substitutions. Also contemplated herein are isomaltase enzymes comprising the foregoing substitutions at the corresponding positions in SEQ ID NO: 1, namely wild-type isomaltase enzyme derived from L. fermentum (see TABLE 1 below).

In certain embodiments, the isomaltase comprises: a substitution of a K residue at a position corresponding to position 115 of wild-type L. fermentum isomaltase (SEQ ID NO: 1); a substitution of a R residue at a position corresponding to position 132 of wild-type L. fermentum isomaltase (SEQ ID NO: 1); a substitution of a D residue at a position corresponding to position 226 of wild-type L. fermentum isomaltase (SEQ ID NO: 1); a substitution of an E residue at a position corresponding to position 310 of wild-type L. fermentum isomaltase (SEQ ID NO: 1); a substitution of a L residue at a position corresponding to position 366 of wild-type L. fermentum isomaltase (SEQ ID NO: 1); a substitution of a I residue at a position corresponding to position 421 of wild-type L. fermentum isomaltase (SEQ ID NO: 1); a substitution of an A residue at a position corresponding to position 444 of wild-type L. fermentum isomaltase (SEQ ID NO: 1); a substitution of an A residue at a position corresponding to position 531 of wild-type L. fermentum isomaltase (SEQ ID NO: 1); a substitution of a F residue at a position corresponding to position 560 of wild-type L. fermentum isomaltase (SEQ ID NO: 1); a substitution of a T residue at a position corresponding to position 82 of wild-type L. fermentum isomaltase (SEQ ID NO: 1); a substitution of an E residue at a position corresponding to position 86 of wild-type L. fermentum isomaltase (SEQ ID NO: 1); a substitution of a T residue at a position corresponding to position 89 of wild-type L. fermentum isomaltase (SEQ ID NO: 1); a substitution of an E residue at a position corresponding to position 93 of wild-type L. fermentum isomaltase (SEQ ID NO: 1); a substitution of an E residue at a position corresponding to position 122 of wild-type L. fermentum isomaltase (SEQ ID NO: 1); a substitution of a D residue at a position corresponding to position 136 of wild-type L. fermentum isomaltase (SEQ ID NO: 1); a substitution of a T residue at a position corresponding to position 157 of wild-type L. fermentum isomaltase (SEQ ID NO: 1); a substitution of a V residue at a position corresponding to position 168 of wild-type L. fermentum isomaltase (SEQ ID NO: 1); a substitution of an E residue at a position corresponding to position 178 of wild-type L. fermentum isomaltase (SEQ ID NO: 1); a substitution of a H residue at a position corresponding to position 182 of wild-type L. fermentum isomaltase (SEQ ID NO: 1); a substitution of a D residue at a position corresponding to position 186 of wild-type L. fermentum isomaltase (SEQ ID NO: 1); a substitution of an A residue at a position corresponding to position 211 of wild-type L. fermentum isomaltase (SEQ ID NO: 1); a substitution of a Q residue at a position corresponding to position 246 of wild-type L. fermentum isomaltase (SEQ ID NO: 1); a substitution of a K residue at a position corresponding to position 269 of wild-type L. fermentum isomaltase (SEQ ID NO: 1); a substitution of an E residue at a position corresponding to position 275 of wild-type L. fermentum isomaltase (SEQ ID NO: 1); a substitution of a P residue at a position corresponding to position 309 of wild-type L. fermentum isomaltase (SEQ ID NO: 1); a substitution of a L residue at a position corresponding to position 394 of wild-type L. fermentum isomaltase (SEQ ID NO: 1); a substitution of a S residue at a position corresponding to position 455 of wild-type L. fermentum isomaltase (SEQ ID NO: 1); a substitution of a M residue at a position corresponding to position 486 of wild-type L. fermentum isomaltase (SEQ ID NO: 1); a substitution of a E residue at a position corresponding to position 498 of wild-type L. fermentum isomaltase (SEQ ID NO: 1); a substitution of an E residue at a position corresponding to position 501 of wild-type L. fermentum isomaltase (SEQ ID NO: 1); a substitution of a Q residue at a position corresponding to position 502 of wild-type L. fermentum isomaltase (SEQ ID NO: 1); a substitution of a T residue at a position corresponding to position 514 of wild-type L. fermentum isomaltase (SEQ ID NO: 1); a substitution of an E residue at a position corresponding to position 524 of wild-type L. fermentum isomaltase (SEQ ID NO: 1); a substitution of a G residue at a position corresponding to position 533 of wild-type L. fermentum isomaltase (SEQ ID NO: 1); a substitution of a G residue at a position corresponding to position 535 of wild-type L. fermentum isomaltase (SEQ ID NO: 1); a substitution of an E residue at a position corresponding to position 543 of wild-type L. fermentum isomaltase (SEQ ID NO: 1); a substitution of a G residue at a position corresponding to position 547 of wild-type L. fermentum isomaltase (SEQ ID NO: 1); a substitution of a R residue at a position corresponding to position 551 of wild-type L. fermentum isomaltase (SEQ ID NO: 1); a substitution of an E residue at a position corresponding to position 554 of wild-type L. fermentum isomaltase (SEQ ID NO: 1); a substitution of a K residue at a position corresponding to position 556 of wild-type L. fermentum isomaltase (SEQ ID NO: 1); or a combination of any of the foregoing substitutions.

In certain embodiments, the isomaltase comprises: the K residue at a position corresponding to position 115 of wild-type L. fermentum isomaltase is substituted by I (K115I); the R residue at a position corresponding to position 132 of wild-type L. fermentum isomaltase is substituted by K (R132K); the D residue at a position corresponding to position 226 of wild-type L. fermentum isomaltase is substituted by S (D226S); the E residue at a position corresponding to position 310 of wild-type L. fermentum isomaltase is substituted by A (E310A); the L residue at a position corresponding to position 366 of wild-type L. fermentum isomaltase is substituted by M (L366M); the I residue at a position corresponding to position 421 of wild-type L. fermentum isomaltase is substituted by A (I421A); the A residue at a position corresponding to position 444 of wild-type L. fermentum isomaltase is substituted by G (A444G); the A residue at a position corresponding to position 531 of wild-type L. fermentum isomaltase is substituted by D (A531D); the F residue at a position corresponding to position 560 of wild-type L. fermentum isomaltase is substituted by V (F560V); the S residue at a position corresponding to position 12 of wild-type L. fermentum isomaltase is substituted by A (S12A); the T residue at a position corresponding to position 82 of wild-type L. fermentum isomaltase is substituted by A (T82A); the E residue at a position corresponding to position 86 of wild-type L. fermentum isomaltase is substituted by Q (E86Q); the T residue at a position corresponding to position 89 of wild-type L. fermentum isomaltase is substituted by A (T89A); the E residue at a position corresponding to position 93 of wild-type L. fermentum isomaltase is substituted by K (E93K); the E residue at a position corresponding to position 122 of wild-type L. fermentum isomaltase is substituted by D (E122D); the D residue at a position corresponding to position 136 of wild-type L. fermentum isomaltase is substituted by N (D136N); the T residue at a position corresponding to position 157 of wild-type L. fermentum isomaltase is substituted by A (T157A); the V residue at a position corresponding to position 168 of wild-type L. fermentum isomaltase is substituted by K (V168K); the E residue at a position corresponding to position 178 of wild-type L. fermentum isomaltase is substituted by D (E178D); the E residue at a position corresponding to position 178 of wild-type L. fermentum isomaltase is substituted by P (E178P); the H residue at a position corresponding to position 182 of wild-type L. fermentum isomaltase is substituted by R (H182R); the D residue at a position corresponding to position 186 of wild-type L. fermentum isomaltase is substituted by E (D186E); the A residue at a position corresponding to position 211 of wild-type L. fermentum isomaltase is substituted by E (A211E); the Q residue at a position corresponding to position 246 of wild-type L. fermentum isomaltase is substituted by R (Q246R); the K residue at a position corresponding to position 269 of wild-type L. fermentum isomaltase is substituted by R (K269R); the E residue at a position corresponding to position 275 of wild-type L. fermentum isomaltase is substituted by D (E275D); the P residue at a position corresponding to position 309 of wild-type L. fermentum isomaltase is substituted by R (P309R); the L residue at a position corresponding to position 394 of wild-type L. fermentum isomaltase is substituted by I (L394I); the S residue at a position corresponding to position 455 of wild-type L. fermentum isomaltase is substituted by A (S455A); the M residue at a position corresponding to position 486 of wild-type L. fermentum isomaltase is substituted by I (M486I); the E residue at a position corresponding to position 498 of wild-type L. fermentum isomaltase is substituted by N (E498N); the E residue at a position corresponding to position 498 of wild-type L. fermentum isomaltase is substituted by D (E498D); the E residue at a position corresponding to position 501 of wild-type L. fermentum isomaltase is substituted by D (E501D); the Q residue at a position corresponding to position 502 of wild-type L. fermentum isomaltase is substituted by E (Q502E); the T residue at a position corresponding to position 514 of wild-type L. fermentum isomaltase is substituted by K (T514K); the E residue at a position corresponding to position 524 of wild-type L. fermentum isomaltase is substituted by Q (E524Q); the G residue at a position corresponding to position 533 of wild-type L. fermentum isomaltase is substituted by E (G533E); the G residue at a position corresponding to position 535 of wild-type L. fermentum isomaltase is substituted by S (G535S); the E residue at a position corresponding to position 543 of wild-type L. fermentum isomaltase is substituted by D (E543D); the E residue at a position corresponding to position 543 of wild-type L. fermentum isomaltase is substituted by N (E543N); the G residue at a position corresponding to position 547 of wild-type L. fermentum isomaltase is substituted by S (G547S); the R residue at a position corresponding to position 551 of wild-type L. fermentum isomaltase is substituted by N (R551N); the E residue at a position corresponding to position 554 of wild-type L. fermentum isomaltase is substituted by Q (E554Q); the K residue at a position corresponding to position 556 of wild-type L. fermentum isomaltase is substituted by I (K556I); the F residue at a position corresponding to position 560 of wild-type L. fermentum isomaltase is substituted by I (F560I); the F residue at a position corresponding to position 560 of wild-type L. fermentum isomaltase is substituted by L (F560L); or the isomaltase comprises a combination of any of the foregoing substitutions.

In certain embodiments, the isomaltase comprises one, two, three, four, five, six, seven, eight, nine, ten, or more than ten mutations relative to the corresponding wild-type L. fermentum isomaltase (SEQ ID NO:1).

In certain embodiments, the recombinant mutant isomaltase comprises an amino acid sequence comprising: (a) at least one substitution relative to SEQ ID NO: 1 selected from the group consisting of K115I, R132K, A444G and A531D; (b) the amino acid substitutions K115I, R132K, A444G and A531D relative to SEQ ID NO: 1; (c) at least one substitution selected from the group consisting of K115I, D226S, I421A, A444G, and A531D relative to SEQ ID NO: 1; (d) the amino acid substitutions K115I, D226S, I421A, A444G, and A531D, and optionally further comprises either F560V or F560L relative to SEQ ID NO: 1; (e) the amino acid substitutions K115I, D226S, I421A, A444G, and A531D, and optionally further comprises one or more substitutions selected from the group consisting of T89A, E93K, E122D, R132K, H182R, A211E, E275D, E310A, L366M, and E524Q relative to SEQ ID NO. 1; (f) an amino acid sequence of SEQ. ID NO: 1 with at least the amino acid substitutions K115I, D226S, I421A, A444G, and A531D; or (g) an amino acid sequence of SEQ. ID NO: 1 with at least the amino acid substitutions K115I, D226S, I421A, A444G, A531D, and either F560V or F560L.

In certain embodiments, the recombinant mutant isomaltase comprises at least three substitutions selected from: (a) the T157A, E275D, and L366M substitutions relative to SEQ ID NO: 1; (b) the G533E, E543D, and G547S substitutions relative to SEQ ID NO: 1; (c) the R132K, E498D, A531D, T89A, E178P, and F560L substitutions relative to SEQ ID NO: 1; (d) the R132K, S455A, E498D, A531D, K115I, and D186E substitutions relative to SEQ ID NO: 1; and (e) the R132K, E498D, A531D, T82A, E178P, and E501D substitutions relative to SEQ ID NO: 1.

In certain embodiments, the isomaltase comprises the following substitutions at positions corresponding to the amino acids set forth in SEQ ID NO: 1, including the S12A, T89A, K115I, R132K, E275D, A444G, and A531D substitutions; the T89A, K115I, R132K, A444G, S455A, A531D, and F560I substitutions; the T89A, K115I, R132K, D226S, P309R, A444G, and A531D substitutions; the T82A, K115I, R132K, E275D, A444G, S455A, and A531D substitutions; the K115I, R132K, H182R, A444G, T514K, A531D, and E543N substitutions; the E93K, K115I, T157A, A444G, and A531D substitutions; the K115I, R132K, A211E, K269R, E275D, A444G, and A53 iD substitutions; the E86Q, K115I, R132K, A444G, M486I, A531D, and F560V substitutions; the K115I, R132K, T157A, D226S, A444G, E524Q, and A531D substitutions; the E93K, K115I, R132K, D136N, A444G, A531D, and K556I substitutions; the K115I, R132K, T157A, H182R, A444G, S455A, and A531D substitutions; the K115I, R132K, E275D, A444G, A531D, R551N, and F560V substitutions; the K115I, E275D, P309R, A444G, and A531D substitutions; the K115I, R132K, E178D, L366M, A444G, A531D, and F560I substitutions; the S12A, K115I, R132K, H182R, K269R, A444G, and A531D substitutions; the T89A, E93K, K115I, R132K, H182R, A444G, and A531D substitutions; the K115I, A211E, L366M, A444G, and A531D substitutions; the T82A, K115I, R132K, A444G, Q502E, A531D, and F560L substitutions; the S12A, K115I, R132K, E178D, E310A, A444G, and A531D substitutions; the K115I, R132K, Q246R, A444G, A531D, G535S, and K556I substitutions; the E93K, K115I, R132K, E275D, A444G, A531D, and F560I substitutions; the T82A, E93K, K115I, R132K, L366M, A444G, and A531D substitutions; the E93K, K115I, R132K, V168K, A444G, M486I, and A531D substitutions; the S12A, T82A, K115I, R132K, and A531D substitutions; the E86Q, K115I, R132K, E275D, L366M, A444G, and A531D substitutions; the E93K, K115I, R132K, A444G, and G535S substitutions; the K115I, R132K, D226S, I421A, A444G, A531D, and F560L substitutions; the S12A, E93K, K115I, R132K, A444G, S455A, and A531D substitutions; the K115I, R132K, K269R, A444G, A531D, E554Q, and K556I substitutions; the K115I, E122D, R132K, E310A, A444G, A531D, and E543N substitutions; the S12A, K115I, R132K, D136N, A444G, A531D, and G535S substitutions; the K115I, R132K, E178D, E498N, and A531D substitutions; the K115I, R132K, K269R, A444G, and S455A substitutions; the T82A, K115I, R132K, D226S, L394I, A444G, and A531D substitutions; the K115I, R132K, S455A, A531D, and F560L substitutions; the K115I, R132K, E310A, A444G, Q502E, T514K, and A531D substitutions; the S12A, K115I, R132K, L366M, and A444G substitutions; the T82A, T89A, K115I, R132K, and A444G substitutions; the K115I, R132K, T157A, A444G, A531D, G535S, and F560L substitutions; the T82A, K115I, R132K, Q246R, A444G, A531D, and E543N substitutions; the E93K, K115I, R132K, A211E, A444G, T514K, and A531D substitutions; or the K115I, R132K, E178D, A211E, A444G, A531D, and F560L substitutions.

In certain embodiments, the isomaltase comprises the following substitutions at positions corresponding to the amino acids set forth in SEQ ID NO: 1, including the K115I, R132K, D226S, E310A, L366M, I421A, A444G, A531D, and F560V substitutions; the E93K, K115I, R132K, D226S, E310A, L366M, I421A, A444G, A531D, and F560L substitutions; the E93K, K115I, R132K, D226S, E310A, I421A, A444G, E524Q, A531D, and F560L substitutions; the E93K, K115I, R132K, D226S, E310A, I421A, A444G, A531D, and F560V substitutions; the E93K, K115I, R132K, D226S, L366M, I421A, A444G, E524Q, A531D, and F560L substitutions; the E93K, K115I, R132K, D226S, L366M, I421A, A444G, A531D, and F560V substitutions; the E93K, K115I, R132K, D226S, I421A, A444G, E524Q, A531D, and F560V substitutions; the K115I, R132K, D226S, E310A, L366M, I421A, A444G, E524Q, A531D, and F560L substitutions; the K115I, R132K, D226S, E310A, I421A, A444G, E524Q, A531D, and F560V substitutions; the K115I, R132K, D226S, L366M, I421A, A444G, E524Q, A531D, and F560V substitutions; the E93K, K115I, R132K, A211E, D226S, I421A, A444G, E524Q, A531D, and F560V substitutions; the E93K, K115I, R132K, D226S, L366M, I421A, A444G, E524Q, A531D, and F560V substitutions; the E93K, K115I, R132K, D226S, E275D, I421A, A444G, E524Q, A531D, and F560V substitutions; the T89A, E93K, K115I, R132K, D226S, I421A, A444G, E524Q, A531D, and F560V substitutions; the E93K, K115I, R132K, H182R, D226S, I421A, A444G, E524Q, A531D, and F560V substitutions; the E93K, K115I, E122D, R132K, D226S, I421A, A444G, E524Q, A531D, and F560V substitutions; the E93K, K115I, D226S, I421A, A444G, E524Q, A531D, and F560V substitutions; the E93K, K115I, D226S, I421A, A444G, E524Q, A531D, and F560L substitutions; the K115I, A211E, D226S, I421A, A444G, A531D, and F560V substitutions; the K115I, D226S, L366M, I421A, A444G, E524Q, A531D, and F560L substitutions; the K115I, A211E, D226S, I421A, A444G, E524Q, A531D, and F560L substitutions; the K115I, R132K, D226S, E310A, L366M, I421A, A444G, A531D, and F560L substitutions; the E93K, K115I, D226S, E310A, I421A, A444G, A531D, and F560L substitutions; the K115I, D226S, E310A, I421A, A444G, A531D, and F560V substitutions; the E93K, K115I, D226S, L366M, I421A, A444G, A531D, and F560L substitutions; the K115I, D226S, I421A, A444G, E524Q, A531D, and F560V substitutions; the K115I, D226S, E310A, I421A, A444G, E524Q, A531D, and F560L substitutions; the K115I, A211E, D226S, L366M, I421A, A444G, A531D, and F560L substitutions; the K115I, A211E, D226S, E310A, I421A, A444G, A531D, and F560L substitutions; the E93K, K115I, D226S, I421A, A444G, A531D, and F560V substitutions; the E93K, K115I, A211E, D226S, I421A, A444G, A531D, and F560L substitutions; or the K115I, D226S, L366M, I421A, A444G, A531D, and F560V substitutions.

In certain embodiments, the isomaltase comprises the amino acid sequence of any one of SEQ ID NOs: 7, 1-6, and 8-12, or an amino acid sequence that has at least 85%, 90%, 91%, 92%, 93%, 94%, 95%, 96%, 97%, 98%, or 99% sequence identity to any one of SEQ ID NOs: 7, 1-6, and 8-12. In certain embodiments, the isomaltase enzyme variant comprises at least one amino acid substitution (e.g., 1, 2, 3, 4, 5, 6, 7, 8, 9, 10 amino acid substitutions) and an amino acid sequence that has at least 98%, 98.5%, 99% or 99.5% sequence identity to SEQ ID NO: 1.

In another aspect, the disclosure relates to a recombinant mutant L. fermentum isomaltase enzyme comprising a substitution, or combination of substitutions listed in TABLE 1.

In each of the foregoing aspects and embodiments, the isomaltase has (i) a specific activity at about pH 4 of at least 25, 30, 35, 40, 45, 50, 55, 60, 65, 70, 75, or 80 μmol isomaltose consumed per minute per milligram of isomaltase, (ii) at least 1 fold, 1.5 fold, 2 fold, 2.5 fold, or 3 fold higher activity at about pH 4, compared to a corresponding wild-type isomaltase, (iii) a specific activity at about pH 6.0 of at least 30, 35, 40, 45, 50, 55, 60, 65, 70, 75, 80, 85, 90, 95, 100, 105, 110, or 115 μmol isomaltose consumed per minute per milligram of isomaltase, (iv) at least 1 fold, 1.5 fold, 2 fold, 2.5 fold, or 3 fold higher activity at about pH 6.0, compared to a corresponding wild-type isomaltase, (v) a specific activity at about pH 7.0 of at least 25, 30, 35, 40, 45, 50, 55, 60, 65, 70, 75, 80, 85, 90, or 95 μmol isomaltose consumed per minute per milligram of isomaltase, (vi) at least 0.5 fold, 1 fold, 1.5 fold, 2 fold, 2.5 fold, or 3 fold higher activity at about pH 7.0, compared to a corresponding wild-type isomaltase, (vii) a Tm of at least 50, 51, 52, 53, 54, 55, 56° C., (viii) a Tm that is at least 1, 2, 3, 4, 5, 6, 7, 8, 9, or 10° C. higher than a corresponding wild-type isomaltase, (ix) higher proteolytic stability in the presence of pancreatin or pepsin compared to a corresponding wild-type isomaltase, or (x) a combination of any one of features (i) to (ix).

In another aspect, the disclosure relates to a nucleic acid encoding an isomaltase disclosed herein, an expression vector comprising such a nucleic acid, a cell (e.g., an E. coli host cell or a S. cerevisiae host cell) comprising such an expression vector. In another aspect, the disclosure relates to method of producing a recombinant mutant L. fermentum isomaltase enzyme, the method comprising growing such a host cell under conditions so that the host cell expresses the isomaltase, and purifying the isomaltase.

In another aspect, the disclosure relates to a pharmaceutical composition comprising an isomaltase as described herein, and a pharmaceutically acceptable carrier and/or an excipient. Depending upon the circumstances, the isomaltase can be spray-dried.

In another aspect, the disclosure relates to a pharmaceutical composition comprising an isomaltase enzyme (e.g., a recombinant mutant isomaltases described herein); a separate invertase enzyme; and a pharmaceutically acceptable carrier and/or an excipient. Depending upon the circumstances and the given the pharmaceutical composition of interest, the isomaltase is spray-dried, the invertase is spray-dried, or both the isomaltase and invertase are spray-dried.

In certain embodiments of the pharmaceutical compositions disclosed herein (i) the isomaltase is a microbial isomaltase (e.g., derived from L. fermentum), or a functional fragment or variant thereof, (ii) the isomaltase comprises a sequence of any one of SEQ ID NOs: 7, 1-6, and 8-12, or a functional fragment or variant thereof, (iii) the isomaltase is a recombinant mutant L. fermentum isomaltase as described herein, (iv) the invertase is a microbial invertase (e.g., derived from Saccharomyces cerevisiae), or a functional fragment or variant thereof, (v) the invertase comprises any one of SEQ ID NOs: 16-17, or a functional fragment or variant thereof, (vi) the invertase comprises any one of SEQ ID NOs: 13-14, or a functional fragment or variant thereof, (vii) the invertase comprises any one of SEQ ID NOs: 19-20, or a functional fragment or variant thereof, (viii) the invertase is a recombinant mutant invertase set forth in U.S. Provisional Patent Application No. 63/388,810, filed on Jul. 13, 2022, or (ix) or a combination of any one of features (i)-(viii).

In certain embodiments, the pharmaceutical compositions described herein are formulated as an oral dosage form (e.g., formulated as a powder, satchel, granulate, pellet, micropellet, tablet, or minitablet). Under certain circumstances, the pharmaceutical composition has a shelf-life at room temperature of at least 3 months, 6 months, 9 months, 12 months, 15 months, 18 months, 21 months, 24 months, 36 months, 48 months, 60 months, 72 months, 84 months, 96 months, 108 months, or 120 months.

In another aspect, the disclosure relates to a method of treating congenital sucrase-isomaltase deficiency (CSID) in a subject (e.g., a mammal, e.g., a human) in need thereof, the method comprising administering (e.g., orally administering) to the subject an enzyme or pharmaceutical composition as described herein. The enzyme or pharmaceutical composition is administered to the subject together with a meal or snack.

In another aspect, the disclosure relates to a method of reducing sucrose and branched (1-6 linked) α-limit dextrin concentration in a subject (e.g., a mammal, e.g., a human), the method comprising administering (e.g., orally administering) to the subject an enzyme or pharmaceutical composition as described herein. Upon administration the enzyme or pharmaceutical composition reduces sucrose and/or branched (1-6 linked) α-limit dextrin concentration in the subject as measured by a hydrogen breath test, e.g., a sucrose methane hydrogen breath test or a ¹³C-sucrose breath test.

In another aspect, the disclosure relates to a method of treating congenital sucrase-isomaltase deficiency (CSID) in a subject (e.g., a mammal, e.g., a human) in need thereof, the method comprising administering (e.g., orally administering) to the subject (i) an isomaltase enzyme and (ii) a separate invertase enzyme. The isomaltase can be a recombinant mutant L. fermentum isomaltase enzyme disclosed herein.

These and other aspects and features of the invention are described in the following detailed description and claims.

### DETAILED DESCRIPTION

The invention is based, in part, upon the development of recombinant mutant isomaltase (α-glucosidase) enzymes that are active in humans and have greater stability and/or activity than naturally occurring enzymes. In particular, the recombinant mutant enzymes disclosed herein may exhibit improved pH stability, thermal stability, and/or stability against proteolytic digestion compared to naturally occurring versions of the enzyme. Furthermore, the recombinant mutant enzymes disclosed herein can have greater specific activity than a wild type isomaltase (α-glucosidase) enzyme. Furthermore, it is contemplated that the recombinant mutant enzymes disclosed herein, given their enhanced stability, may be suitable for oral administration, and potentially safer, more tolerable, and/or more effective than commercially available isomaltase (α-glucosidase) enzymes. The invention is also based, in part, upon the development of pharmaceutical compositions, e.g., solid pharmaceutical compositions, comprising an isomaltase (α-glucosidase) enzyme (e.g., a recombinant isomaltase enzyme disclosed herein) and an optional sucrase (invertase) enzyme that are stable at room temperature, allowing for easier distribution, storage, and administration to subjects relative to compositions that are unstable at room temperature and/or require refrigeration. The recombinant enzymes and compositions can be used, among other things, to treat congenital sucrase-isomaltase deficiency (CSID).

Various features and aspects of the invention are discussed in detail below.

### I. Engineered Isomaltase Enzymes

Among other things, provided herein are recombinant, engineered isomaltase enzymes (enzyme variants or muteins) and pharmaceutical compositions comprising such recombinant mutant isomaltase enzymes that, for example, are useful in treating certain disorders such as congenital sucrase-isomaltase deficiency (CSID).

As used herein, the term “isomaltase” refers to any enzyme, or a functional fragment thereof, that is capable of catalyzing the cleavage of branched (1-6 linked) α-limit dextrins (starches), and isomaltase. Isomaltase enzymes are also called α-glucosidases, alpha-glucosidases, oligo-1,6-glucosidases, and EC 3.2.1.10, and, unless indicated otherwise, the terms are used interchangeably herein. The term isomaltase includes variants having one or more amino acid substitutions, deletions, or insertions relative to a wild-type isomaltase sequence, and/or fusion proteins or conjugates including an isomaltase. As used herein, the term “functional fragment” of an isomaltase refers to fragment of a full-length isomaltase that retains, for example, at least 10%, at least 20%, at least 30%, at least 40%, at least 50%, at least 60%, at least 70%, at least 80%, at least 85%, at least 90%, at least 95% or 100% of the enzymatic activity of the corresponding full-length, naturally occurring isomaltase. Exemplary isomaltase activity assays are described in Yamamoto et al. (2004) EURO. J. BIOCHEM. 271:3414-3420 and also in Example 1B hereinbelow. Additionally, because glucose is a product of the enzymatic reactions catalyzed by both invertase and isomaltase, it is understood that an assay based on the detection of glucose may in certain embodiments be used to measure the activity of both an invertase enzyme and an isomaltase enzyme.

Exemplary isomaltase enzymes include recombinant isomaltase enzyme variants derived from, for example, Limosilactobacillus fermentum (formerly Lactobacillus fermentum, ID: EQC59045.1, L. fermentum MTCC 8711). The amino acid sequence of an exemplary wild-type isomaltase enzyme derived from L. fermentum is set forth in SEQ ID NO: 1, also referred to as V014 herein, and a nucleotide sequence encoding an exemplary wild-type isomaltase enzyme derived from L. fermentum is depicted in SEQ ID NO: 2.

SEQ ID NO: 1
MIYTPKWWWQNSVVYQVYPRSFQDSNHDGIGDLKGIISRLDYIKKLGAD
IIWLNPIYRSPNVDNGYDISDYRAIDPTFGSLTDFKELLTKAHELGLKI
MMDLVVNHSSDENEWFKQSRQGKENPYRDYYIWRDPVDGHEPTNWGSYF
SGSAWQYDETSGQYYLHLFAVKQPDLNWENEAVRHSVYDIMNWWADLGV
DGFRMDVINLISKPAVYKDVPTAPGMQYGDVEPVVANGHRMHEFLQEMH
QAVMAKHDLVTVGETPGATTDDAKKYANLEQTELNMVFEFEHVGLDGND
NPALGKWSDKKVSLPELRDNLVKWQTQLNGKAWNSLYWNNHDQPRVVSR
FGNDDPKYRVVSAKMLATMLHCLQGTPYIYAGEELGMTNTTFNSLSDYR
DLESINAYHQLVDEEHLVDGKTMSRYLAIHSRDNARTPMQWDDSKNAGE
SDAEPWIAVNPNYSEINAKAALADPSSVFYHYQKLIQMRHDLPVMTEGK
FALVNGNELDEQVFAYTRDDGETTLLVVANFTKETIKREYAAGQGKLLL
SNYEDDMGETLRPYEAKVYEFSSKR

An exemplary isomaltase enzyme variant may comprise an amino acid sequence that has at least 80%, 85%, 90%, 91%, 92%, 93%, 94%, 95%, 96%, 97%, 98%, or 99% sequence identity to SEQ ID NO: 1. In certain embodiments, the isomaltase enzyme variant comprises at least one amino acid substitution (e.g., 1, 2, 3, 4, 5, 6, 7, 8, 9, 10 amino acid substitutions, e.g., 1 to 10, 2 to 10, 3 to 10, 4 to 10, 5 to 10, 6 to 10, 1 to 9, 1 to 8, 1 to 7, 1 to 6, 1 to 5, 1 to 4, 2 to 9, 2 to 8, 2 to 7, 2 to 6, 2 to 5 or 2 to 4 amino acid substitutions) and an amino acid sequence that has at least 98%, 98.5%, 99% or 99.5% sequence identity to SEQ ID NO: 1.

Among other things, provided are recombinant isomaltase enzyme variants that are useful, for example, in treating disorders associated with a reduced ability to digest or absorb dietary sucrose and starches.

In certain embodiments, the isomaltase comprises: (i) increased activity at acidic pH (e.g., about pH 4.0 and 6.0) relative to a corresponding wild-type isomaltase; (ii) increased activity at neutral pH (e.g., about pH 7.0) relative to a corresponding wild-type isomaltase; (iii) increased stability at acidic pH (e.g., about pH 4.0) relative to a corresponding wild-type isomaltase, (iv) increased thermal stability relative to a corresponding wild-type isomaltase, (v) increased resistance to pepsin, relative to a corresponding wild-type isomaltase, (vi) increased resistance to pancreatin relative to a corresponding wild-type isomaltase, and (vii) a combination of any of features (i)-(vi).

In certain embodiments, the recombinant mutant isomaltase comprises at least one (e.g., one, two, three, four, five, six, seven, eight, nine or ten, 2-10, 3-10, 4-10, 5-10, 6-10, 7-10, 8-10, 9-10, 2-9, 3-9, 4-9, 5-9, 6-9, 7-9, 8-9, 2-8, 3-8, 4-8, 5-8, 6-8, 7-8, 2-6, 3-6, 4-6, or 5-6) mutation(s) at a position corresponding to wild type L. fermentum isomaltase of SEQ ID NO: 1. Throughout the specification a single letter code for amino acids are used, which include alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y), and valine (V).

In certain embodiments, at least one mutation is selected from a substitution of a residue at a position corresponding to position 12 of wild-type L. fermentum isomaltase; a substitution of a residue at a position corresponding to position 82 of wild-type L. fermentum isomaltase; a substitution of a residue at a position corresponding to position 86 of wild-type L. fermentum isomaltase; a substitution of a residue at a position corresponding to position 89 of wild-type L. fermentum isomaltase; a substitution of a residue at a position corresponding to position 93 of wild-type L. fermentum isomaltase; a substitution of a residue at a position corresponding to position 115 of wild-type L. fermentum isomaltase; a substitution of a residue at a position corresponding to position 122 of wild-type L. fermentum isomaltase; a substitution of a residue at a position corresponding to position 132 of wild-type L. fermentum isomaltase; a substitution of a residue at a position corresponding to position 136 of wild-type L. fermentum isomaltase; a substitution of a residue at a position corresponding to position 157 of wild-type L. fermentum isomaltase; a substitution of a residue at a position corresponding to position 168 of wild-type L. fermentum isomaltase; a substitution of a residue at a position corresponding to position 178 of wild-type L. fermentum isomaltase; a substitution of a residue at a position corresponding to position 182 of wild-type L. fermentum isomaltase; a substitution of a residue at a position corresponding to position 186 of wild-type L. fermentum isomaltase; a substitution of a residue at a position corresponding to position 211 of wild-type L. fermentum isomaltase; a substitution of a residue at a position corresponding to position 226 of wild-type L. fermentum isomaltase; a substitution of a residue at a position corresponding to position 246 of wild-type L. fermentum isomaltase; a substitution of a residue at a position corresponding to position 269 of wild-type L. fermentum isomaltase; a substitution of a residue at a position corresponding to position 275 of wild-type L. fermentum isomaltase; a substitution of a residue at a position corresponding to position 309 of wild-type L. fermentum isomaltase; a substitution of a residue at a position corresponding to position 310 of wild-type L. fermentum isomaltase; a substitution of a residue at a position corresponding to position 366 of wild-type L. fermentum isomaltase; a substitution of a residue at a position corresponding to position 394 of wild-type L. fermentum isomaltase; a substitution of a residue at a position corresponding to position 421 of wild-type L. fermentum isomaltase; a substitution of a residue at a position corresponding to position 444 of wild-type L. fermentum isomaltase; a substitution of a residue at a position corresponding to position 455 of wild-type L. fermentum isomaltase; a substitution of a residue at a position corresponding to position 486 of wild-type L. fermentum isomaltase; a substitution of a residue at a position corresponding to position 498 of wild-type L. fermentum isomaltase; a substitution of a residue at a position corresponding to position 501 of wild-type L. fermentum isomaltase; a substitution of a residue at a position corresponding to position 502 of wild-type L. fermentum isomaltase; a substitution of a residue at a position corresponding to position 514 of wild-type L. fermentum isomaltase; a substitution of a residue at a position corresponding to position 524 of wild-type L. fermentum isomaltase; a substitution of a residue at a position corresponding to position 531 of wild-type L. fermentum isomaltase; a substitution of a residue at a position corresponding to position 533 of wild-type L. fermentum isomaltase; a substitution of a residue at a position corresponding to position 535 of wild-type L. fermentum isomaltase; a substitution of a residue at a position corresponding to position 543 of wild-type L. fermentum isomaltase; a substitution of a residue at a position corresponding to position 554 of wild-type L. fermentum isomaltase; a substitution of a residue at a position corresponding to position 556 of wild-type L. fermentum isomaltase; or a substitution of a residue at a position corresponding to position 560 of wild-type L. fermentum isomaltase, wherein the wild-type L. fermentum isomaltase comprises the amino acid sequence of SEQ ID NO:1. In certain embodiments, the recombinant mutant isomaltase comprises a combination of any of the foregoing substitutions.

In certain embodiments, the isomaltase comprises: the residue at a position corresponding to position 12 of wild-type L. fermentum isomaltase is substituted by A; the residue at a position corresponding to position 82 of wild-type L. fermentum isomaltase is substituted by A; the residue at a position corresponding to position 86 of wild-type L. fermentum isomaltase is substituted by Q; the residue at a position corresponding to position 89 of wild-type L. fermentum isomaltase is substituted by A; the residue at a position corresponding to position 93 of wild-type L. fermentum isomaltase is substituted by K; the residue at a position corresponding to position 115 of wild-type L. fermentum isomaltase is substituted by I; the residue at a position corresponding to position 122 of wild-type L. fermentum isomaltase is substituted by D; the residue at a position corresponding to position 132 of wild-type L. fermentum isomaltase is substituted by K; the residue at a position corresponding to position 136 of wild-type L. fermentum isomaltase is substituted by N; the residue at a position corresponding to position 157 of wild-type L. fermentum isomaltase is substituted by A; the residue at a position corresponding to position 168 of wild-type L. fermentum isomaltase is substituted by K; the residue at a position corresponding to position 178 of wild-type L. fermentum isomaltase is substituted by D; the residue at a position corresponding to position 178 of wild-type L. fermentum isomaltase is substituted by P; the residue at a position corresponding to position 182 of wild-type L. fermentum isomaltase is substituted by R; the residue at a position corresponding to position 186 of wild-type L. fermentum isomaltase is substituted by E; the residue at a position corresponding to position 211 of wild-type L. fermentum isomaltase is substituted by E; the residue at a position corresponding to position 226 of wild-type L. fermentum isomaltase is substituted by S; the residue at a position corresponding to position 246 of wild-type L. fermentum isomaltase is substituted by R; the residue at a position corresponding to position 269 of wild-type L. fermentum isomaltase is substituted by R; the residue at a position corresponding to position 275 of wild-type L. fermentum isomaltase is substituted by D; the residue at a position corresponding to position 309 of wild-type L. fermentum isomaltase is substituted by R; the residue at a position corresponding to position 310 of wild-type L. fermentum isomaltase is substituted by A; the residue at a position corresponding to position 366 of wild-type L. fermentum isomaltase is substituted by M; the residue at a position corresponding to position 394 of wild-type L. fermentum isomaltase is substituted by I; the residue at a position corresponding to position 421 of wild-type L. fermentum isomaltase is substituted by A; the residue at a position corresponding to position 444 of wild-type L. fermentum isomaltase is substituted by G; the residue at a position corresponding to position 455 of wild-type L. fermentum isomaltase is substituted by A; the residue at a position corresponding to position 486 of wild-type L. fermentum isomaltase is substituted by I; the residue at a position corresponding to position 498 of wild-type L. fermentum isomaltase is substituted by N; the residue at a position corresponding to position 498 of wild-type L. fermentum isomaltase is substituted by D; the residue at a position corresponding to position 501 of wild-type L. fermentum isomaltase is substituted by D; the residue at a position corresponding to position 502 of wild-type L. fermentum isomaltase is substituted by E; the residue at a position corresponding to position 514 of wild-type L. fermentum isomaltase is substituted by K; the residue at a position corresponding to position 524 of wild-type L. fermentum isomaltase is substituted by Q; the residue at a position corresponding to position 531 of wild-type L. fermentum isomaltase is substituted by D; the residue at a position corresponding to position 533 of wild-type L. fermentum isomaltase is substituted by E; the residue at a position corresponding to position 535 of wild-type L. fermentum isomaltase is substituted by S; the residue at a position corresponding to position 543 of wild-type L. fermentum isomaltase is substituted by D; the residue at a position corresponding to position 543 of wild-type L. fermentum isomaltase is substituted by N; the residue at a position corresponding to position 547 of wild-type L. fermentum isomaltase is substituted by S; the residue at a position corresponding to position 551 of wild-type L. fermentum isomaltase is substituted by N; the residue at a position corresponding to position 554 of wild-type L. fermentum isomaltase is substituted by Q; the residue at a position corresponding to position 556 of wild-type L. fermentum isomaltase is substituted by I; the residue at a position corresponding to position 560 of wild-type L. fermentum isomaltase is substituted by I; the residue at a position corresponding to position 560 of wild-type L. fermentum isomaltase is substituted by L; or the residue at a position corresponding to position 560 of wild-type L. fermentum isomaltase is substituted by V. In certain embodiments, the recombinant mutant isomaltase comprises a combination of any of the foregoing substitutions.

In certain embodiments, the isomaltase comprises: a substitution of a T residue at a position corresponding to position 82 of wild-type L. fermentum isomaltase (SEQ ID NO: 1); a substitution of an E residue at a position corresponding to position 86 of wild-type L. fermentum isomaltase (SEQ ID NO: 1); a substitution of a T residue at a position corresponding to position 89 of wild-type L. fermentum isomaltase (SEQ ID NO: 1); a substitution of an E residue at a position corresponding to position 93 of wild-type L. fermentum isomaltase (SEQ ID NO: 1); a substitution of a K residue at a position corresponding to position 115 of wild-type L. fermentum isomaltase (SEQ ID NO: 1); a substitution of an E residue at a position corresponding to position 122 of wild-type L. fermentum isomaltase (SEQ ID NO: 1); a substitution of a R residue at a position corresponding to position 132 of wild-type L. fermentum isomaltase (SEQ ID NO: 1); a substitution of a D residue at a position corresponding to position 136 of wild-type L. fermentum isomaltase (SEQ ID NO: 1); a substitution of a T residue at a position corresponding to position 157 of wild-type L. fermentum isomaltase (SEQ ID NO: 1); a substitution of a V residue at a position corresponding to position 168 of wild-type L. fermentum isomaltase (SEQ ID NO: 1); a substitution of an E residue at a position corresponding to position 178 of wild-type L. fermentum isomaltase (SEQ ID NO: 1); a substitution of a H residue at a position corresponding to position 182 of wild-type L. fermentum isomaltase (SEQ ID NO: 1); a substitution of a D residue at a position corresponding to position 186 of wild-type L. fermentum isomaltase (SEQ ID NO: 1); a substitution of an A residue at a position corresponding to position 211 of wild-type L. fermentum isomaltase (SEQ ID NO: 1); a substitution of a D residue at a position corresponding to position 226 of wild-type L. fermentum isomaltase (SEQ ID NO: 1); a substitution of a Q residue at a position corresponding to position 246 of wild-type L. fermentum isomaltase (SEQ ID NO: 1); a substitution of a K residue at a position corresponding to position 269 of wild-type L. fermentum isomaltase (SEQ ID NO: 1); a substitution of an E residue at a position corresponding to position 275 of wild-type L. fermentum isomaltase (SEQ ID NO: 1); a substitution of a P residue at a position corresponding to position 309 of wild-type L. fermentum isomaltase (SEQ ID NO: 1); a substitution of an E residue at a position corresponding to position 310 of wild-type L. fermentum isomaltase (SEQ ID NO: 1); a substitution of a L residue at a position corresponding to position 366 of wild-type L. fermentum isomaltase (SEQ ID NO: 1); a substitution of a L residue at a position corresponding to position 394 of wild-type L. fermentum isomaltase (SEQ ID NO: 1); a substitution of a I residue at a position corresponding to position 421 of wild-type L. fermentum isomaltase (SEQ ID NO: 1); a substitution of an A residue at a position corresponding to position 444 of wild-type L. fermentum isomaltase (SEQ ID NO: 1); a substitution of a S residue at a position corresponding to position 455 of wild-type L. fermentum isomaltase (SEQ ID NO: 1); a substitution of a M residue at a position corresponding to position 486 of wild-type L. fermentum isomaltase (SEQ ID NO: 1); a substitution of a E residue at a position corresponding to position 498 of wild-type L. fermentum isomaltase (SEQ ID NO: 1); a substitution of an E residue at a position corresponding to position 501 of wild-type L. fermentum isomaltase (SEQ ID NO: 1); a substitution of a Q residue at a position corresponding to position 502 of wild-type L. fermentum isomaltase (SEQ ID NO: 1); a substitution of a T residue at a position corresponding to position 514 of wild-type L. fermentum isomaltase (SEQ ID NO: 1); a substitution of an E residue at a position corresponding to position 524 of wild-type L. fermentum isomaltase (SEQ ID NO: 1); a substitution of an A residue at a position corresponding to position 531 of wild-type L. fermentum isomaltase (SEQ ID NO: 1); a substitution of a G residue at a position corresponding to position 533 of wild-type L. fermentum isomaltase (SEQ ID NO: 1); a substitution of a G residue at a position corresponding to position 535 of wild-type L. fermentum isomaltase (SEQ ID NO: 1); a substitution of an E residue at a position corresponding to position 543 of wild-type L. fermentum isomaltase (SEQ ID NO: 1); a substitution of a G residue at a position corresponding to position 547 of wild-type L. fermentum isomaltase (SEQ ID NO: 1); a substitution of a R residue at a position corresponding to position 551 of wild-type L. fermentum isomaltase (SEQ ID NO: 1); a substitution of an E residue at a position corresponding to position 554 of wild-type L. fermentum isomaltase (SEQ ID NO: 1); a substitution of a K residue at a position corresponding to position 556 of wild-type L. fermentum isomaltase (SEQ ID NO: 1); or a substitution of a F residue at a position corresponding to position 560 of wild-type L. fermentum isomaltase (SEQ ID NO: 1). In certain embodiments, the recombinant mutant isomaltase comprises a combination of any of the foregoing substitutions.

In certain embodiments, the isomaltase comprises: the S residue at a position corresponding to position 12 of wild-type L. fermentum isomaltase is substituted by A (S12A); the T residue at a position corresponding to position 82 of wild-type L. fermentum isomaltase is substituted by A (T82A); the E residue at a position corresponding to position 86 of wild-type L. fermentum isomaltase is substituted by Q (E86Q); the T residue at a position corresponding to position 89 of wild-type L. fermentum isomaltase is substituted by A (T89A); the E residue at a position corresponding to position 93 of wild-type L. fermentum isomaltase is substituted by K (E93K); the K residue at a position corresponding to position 115 of wild-type L. fermentum isomaltase is substituted by I (K115I); the E residue at a position corresponding to position 122 of wild-type L. fermentum isomaltase is substituted by D (E122D); the R residue at a position corresponding to position 132 of wild-type L. fermentum isomaltase is substituted by K (R132K); the D residue at a position corresponding to position 136 of wild-type L. fermentum isomaltase is substituted by N (D136N); the T residue at a position corresponding to position 157 of wild-type L. fermentum isomaltase is substituted by A (T157A); the V residue at a position corresponding to position 168 of wild-type L. fermentum isomaltase is substituted by K (V168K); the E residue at a position corresponding to position 178 of wild-type L. fermentum isomaltase is substituted by D (E178D); the E residue at a position corresponding to position 178 of wild-type L. fermentum isomaltase is substituted by P (E178P); the H residue at a position corresponding to position 182 of wild-type L. fermentum isomaltase is substituted by R (H182R); the D residue at a position corresponding to position 186 of wild-type L. fermentum isomaltase is substituted by E (D186E); the A residue at a position corresponding to position 211 of wild-type L. fermentum isomaltase is substituted by E (A211E); the D residue at a position corresponding to position 226 of wild-type L. fermentum isomaltase is substituted by S (D226S); the Q residue at a position corresponding to position 246 of wild-type L. fermentum isomaltase is substituted by R (Q246R); the K residue at a position corresponding to position 269 of wild-type L. fermentum isomaltase is substituted by R (K269R); the E residue at a position corresponding to position 275 of wild-type L. fermentum isomaltase is substituted by D (E275D); the P residue at a position corresponding to position 309 of wild-type L. fermentum isomaltase is substituted by R (P309R); the E residue at a position corresponding to position 310 of wild-type L. fermentum isomaltase is substituted by A (E310A); the L residue at a position corresponding to position 366 of wild-type L. fermentum isomaltase is substituted by M (L366M); the L residue at a position corresponding to position 394 of wild-type L. fermentum isomaltase is substituted by I (L394I); the I residue at a position corresponding to position 421 of wild-type L. fermentum isomaltase is substituted by A (I421A); the A residue at a position corresponding to position 444 of wild-type L. fermentum isomaltase is substituted by G (A444G); the S residue at a position corresponding to position 455 of wild-type L. fermentum isomaltase is substituted by A (S455A); the M residue at a position corresponding to position 486 of wild-type L. fermentum isomaltase is substituted by I (M486I); the E residue at a position corresponding to position 498 of wild-type L. fermentum isomaltase is substituted by N (E498N); the E residue at a position corresponding to position 498 of wild-type L. fermentum isomaltase is substituted by D (E498D); the E residue at a position corresponding to position 501 of wild-type L. fermentum isomaltase is substituted by D (E501D); the Q residue at a position corresponding to position 502 of wild-type L. fermentum isomaltase is substituted by E (Q502E); the T residue at a position corresponding to position 514 of wild-type L. fermentum isomaltase is substituted by K (T514K); the E residue at a position corresponding to position 524 of wild-type L. fermentum isomaltase is substituted by Q (E524Q); the A residue at a position corresponding to position 531 of wild-type L. fermentum isomaltase is substituted by D (A531D); the G residue at a position corresponding to position 533 of wild-type L. fermentum isomaltase is substituted by E (G533E); the G residue at a position corresponding to position 535 of wild-type L. fermentum isomaltase is substituted by S (G535S); the E residue at a position corresponding to position 543 of wild-type L. fermentum isomaltase is substituted by D (E543D); the E residue at a position corresponding to position 543 of wild-type L. fermentum isomaltase is substituted by N (E543N); the G residue at a position corresponding to position 547 of wild-type L. fermentum isomaltase is substituted by S (G547S); the R residue at a position corresponding to position 551 of wild-type L. fermentum isomaltase is substituted by N (R551N); the E residue at a position corresponding to position 554 of wild-type L. fermentum isomaltase is substituted by Q (E554Q); the K residue at a position corresponding to position 556 of wild-type L. fermentum isomaltase is substituted by I (K556I); the F residue at a position corresponding to position 560 of wild-type L. fermentum isomaltase is substituted by I (F560I); the F residue at a position corresponding to position 560 of wild-type L. fermentum isomaltase is substituted by L (F560L); the F residue at a position corresponding to position 560 of wild-type L. fermentum isomaltase is substituted by V (F560V); or the isomaltase comprises a combination of any of the foregoing substitutions. In certain embodiments, the isomaltase comprises one, two, three, four, five, six, seven, eight, nine, ten, or more than ten mutations relative to the corresponding wild-type L. fermentum isomaltase.

In certain embodiments, one or more mutations may be conservative substitutions relative to wild type L. fermentum isomaltase of SEQ ID NO: 1, whereas in certain other embodiments, one or more mutations may be non-conservative substitutions relative to wild type L. fermentum isomaltase of SEQ ID NO: 1. As used herein, the term “conservative substitution” refers to a substitution with a structurally and/or functionally similar amino acid.

In certain embodiments, the substitution of a given amino acid is with a hydrophobic amino acid (e.g., A, I, L, M, or V), a positively charged amino acid (e.g., K, R or H), a negatively charged amino acid (e.g., D or E), a polar neutral amino acid (e.g., N, C, Q, S or T), an aromatic amino acid (e.g., F, Y or W) or a bulkier amino acid based on side chain volume or a smaller amino acid based on side chain volume. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix). Non-conservative substitutions are amino acid substitutions that are not conservative substitutions.

In certain embodiments, the recombinant mutant isomaltase enzyme comprises one or more substitutions from TABLE 1, wherein the positions of the substitutions are shown relative to SEQ ID NO: 1 (wild-type Limosilactobacillus fermentum isomaltase enzyme.

TABLE 1

| Position relative to | Exemplary |
|---|---|
| SEQ ID NO: 1 | Substitutions |
| S12 | A |
| E86 | Q |
| T82 | A |
| T89 | A |
| E93 | K |
| K115 | I |
| E122 | D |
| R132 | K |
| D136 | N |
| T157 | A |
| V168 | K |
| E178 | D, P |
| H182 | R |
| A211 | E |
| D226 | S |
| Q246 | R |
| K269 | R |
| E275 | D |
| P309 | R |
| E310 | A |
| L366 | M |
| L394 | I |
| I421 | A |
| A444 | G |
| S455 | A |
| M486 | I |
| E498 | N |
| E498 | D |
| E501 | D |
| Q502 | E |
| T514 | K |
| E524 | Q |
| A531 | D |
| G533 | E |
| G535 | S |
| E543 | N, D |
| G547 | S |
| R551 | N |
| E554 | Q |
| K556 | I |
| F560 | I, L, and V |

In certain embodiments, the recombinant mutant isomaltase comprises at least one (e.g., at least one, at least two, at least three, at least four, at least five, at least six, at least seven, at least eight, at least nine, at least ten, or at least eleven) mutation(s) selected from TABLE 1.

In certain embodiments, the recombinant mutant isomaltase comprises an amino acid sequence comprising:
(a) at least one substitution relative to SEQ. ID NO: 1 selected from the group consisting of K115I, R132K, A444G, and A531D;
(b) the amino acid substitutions K115I, R132K, A444G, and A531D relative to SEQ ID NO: 1;
(c) at least one substitution selected from the group consisting of K115I, D226S, I421A, A444G, and A531D relative to SEQ ID NO: 1;
(d) the amino acid substitutions K115I, D226S, I421A, A444G, and A531D, and optionally further comprises either F560V or F560L relative to SEQ ID NO: 1;
(e) the amino acid substitutions K115I, D226S, I421A, A444G, and A531D, and optionally further comprises one or more substitutions selected from the group consisting of T89A, E93K, E122D, R132K, H182R, A211E, E275D, E310A, L366M, and E524Q relative to SEQ ID NO. 1;
(f) an amino acid sequence of SEQ. ID NO: 1 with at least the amino acid substitutions K115I, D226S, I421A, A444G, and A531D; or
(g) an amino acid sequence of SEQ. ID NO: 1 with at least the amino acid substitutions K115I, D226S, I421A, A444G, A53 iD, and either F560V or F560L.

In certain embodiments, the recombinant mutant isomaltase comprises at least three substitutions selected from: (a) the T157A, E275D, and L366M substitutions relative to SEQ ID NO: 1; (b) the G533E, E543D, and G547S substitutions relative to SEQ ID NO: 1; (c) the R132K, E498D, A531D, T89A, E178P, and F560L substitutions relative to SEQ ID NO: 1; (d) the R132K, S455A, E498D, A531D, K115I, and D186E substitutions relative to SEQ ID NO: 1; and (e) the R132K, E498D, A531D, T82A, E178P, and E501D substitutions relative to SEQ ID NO: 1.

In certain embodiments, the isomaltase comprises the following substitutions at positions corresponding to the amino acids sequence set forth in SEQ ID NO: 1, including: the S12A, T89A, K115I, R132K, E275D, A444G, and A531D substitutions; the T89A, K115I, R132K, A444G, S455A, A531D, and F560I substitutions; the T89A, K115I, R132K, D226S, P309R, A444G, and A531D substitutions; the T82A, K115I, R132K, E275D, A444G, S455A, and A531D substitutions; the K115I, R132K, H182R, A444G, T514K, A531D, and E543N substitutions; the E93K, K115I, T157A, A444G, and A531D substitutions; the K115I, R132K, A211E, K269R, E275D, A444G, and A531D substitutions; the E86Q, K115I, R132K, A444G, M486I, A531D, and F560V substitutions; the K115I, R132K, T157A, D226S, A444G, E524Q, and A531D substitutions; the E93K, K115I, R132K, D136N, A444G, A531D, and K556I substitutions; the K115I, R132K, T157A, H182R, A444G, S455A, and A531D substitutions; the K115I, R132K, E275D, A444G, A531D, R551N, and F560V substitutions; the K115I, E275D, P309R, A444G, and A531D substitutions; the K115I, R132K, E178D, L366M, A444G, A531D, and F560I substitutions; the S12A, K115I, R132K, H182R, K269R, A444G, and A531D substitutions; the T89A, E93K, K115I, R132K, H182R, A444G, and A531D substitutions; the K115I, A211E, L366M, A444G, and A531D substitutions; the T82A, K115I, R132K, A444G, Q502E, A531D, and F560L substitutions; the S12A, K115I, R132K, E178D, E310A, A444G, and A531D substitutions; the K115I, R132K, Q246R, A444G, A531D, G535S, and K556I substitutions; the E93K, K115I, R132K, E275D, A444G, A531D, and F560I substitutions; the T82A, E93K, K115I, R132K, L366M, A444G, and A531D substitutions; the E93K, K115I, R132K, V168K, A444G, M486I, and A531D substitutions; the S12A, T82A, K115I, R132K, and A531D substitutions; the E86Q, K115I, R132K, E275D, L366M, A444G, and A531D substitutions; the E93K, K115I, R132K, A444G, and G535S substitutions; the K115I, R132K, D226S, I421A, A444G, A531D, and F560L substitutions; the S12A, E93K, K115I, R132K, A444G, S455A, and A531D substitutions; the K115I, R132K, K269R, A444G, A531D, E554Q, and K556I substitutions; the K115I, E122D, R132K, E310A, A444G, A531D, and E543N substitutions; the S12A, K115I, R132K, D136N, A444G, A531D, and G535S substitutions; the K115I, R132K, E178D, E498N, and A531D substitutions; the K115I, R132K, K269R, A444G, and S455A substitutions; the T82A, K115I, R132K, D226S, L394I, A444G, and A531D substitutions; the K115I, R132K, S455A, A531D, and F560L substitutions; the K115I, R132K, E310A, A444G, Q502E, T514K, and A531D substitutions; the S12A, K115I, R132K, L366M, and A444G substitutions; the T82A, T89A, K115I, R132K, and A444G substitutions; the K115I, R132K, T157A, A444G, A531D, G535S, and F560L substitutions; the T82A, K115I, R132K, Q246R, A444G, A531D, and E543N substitutions; the E93K, K115I, R132K, A211E, A444G, T514K, and A531D substitutions; or the K115I, R132K, E178D, A211E, A444G, A531D, and F560L substitutions.

In certain embodiments, the isomaltase comprises the following substitutions at positions corresponding to the amino acids sequence set forth in SEQ ID NO: 1, including: the E93K, K115I, R132K, D226S, E310A, L366M, I421A, A444G, A531D, and F560L substitutions; the E93K, K115I, R132K, D226S, E310A, I421A, A444G, E524Q, A531D, and F560L substitutions; the E93K, K115I, R132K, D226S, E310A, I421A, A444G, A531D, and F560V substitutions; the E93K, K115I, R132K, D226S, L366M, I421A, A444G, E524Q, A531D, and F560L substitutions; the E93K, K115I, R132K, D226S, L366M, I421A, A444G, A531D, and F560V substitutions; the E93K, K115I, R132K, D226S, I421A, A444G, E524Q, A531D, and F560V substitutions; the K115I, R132K, D226S, E310A, L366M, I421A, A444G, E524Q, A531D, and F560L substitutions; the K115I, R132K, D226S, E310A, L366M, I421A, A444G, A531D, and F560V substitutions; the K115I, R132K, D226S, E310A, I421A, A444G, E524Q, A531D, and F560V substitutions; the K115I, R132K, D226S, L366M, I421A, A444G, E524Q, A531D, and F560V substitutions; the E93K, K115I, R132K, A211E, D226S, I421A, A444G, E524Q, A531D, and F560V substitutions; the E93K, K115I, R132K, D226S, L366M, I421A, A444G, E524Q, A531D, and F560V substitutions; the E93K, K115I, R132K, D226S, E275D, I421A, A444G, E524Q, A531D, and F560V substitutions; the T89A, E93K, K115I, R132K, D226S, I421A, A444G, E524Q, A531D, and F560V substitutions; the E93K, K115I, R132K, H182R, D226S, I421A, A444G, E524Q, A531D, and F560V substitutions; the E93K, K115I, E122D, R132K, D226S, I421A, A444G, E524Q, A531D, and F560V substitutions; the E93K, K115I, D226S, I421A, A444G, E524Q, A531D, and F560V substitutions; the E93K, K115I, D226S, I421A, A444G, E524Q, A531D, and F560L substitutions; the K115I, A211E, D226S, I421A, A444G, A531D, and F560V substitutions; the K115I, D226S, L366M, I421A, A444G, E524Q, A531D, and F560L substitutions; the K115I, A211E, D226S, I421A, A444G, E524Q, A531D, and F560L substitutions; the K115I, R132K, D226S, E310A, L366M, I421A, A444G, A531D, and F560L substitutions; the E93K, K115I, D226S, E310A, I421A, A444G, A531D, and F560L substitutions; the K115I, D226S, E310A, I421A, A444G, A531D, and F560V substitutions; the E93K, K115I, D226S, L366M, I421A, A444G, A531D, and F560L substitutions; the K115I, D226S, I421A, A444G, E524Q, A531D, and F560V substitutions; the K115I, D226S, E310A, I421A, A444G, E524Q, A531D, and F560L substitutions; the K115I, A211E, D226S, L366M, I421A, A444G, A531D, and F560L substitutions; the K115I, A211E, D226S, E310A, I421A, A444G, A531D, and F560L substitutions; the E93K, K115I, D226S, I421A, A444G, A531D, and F560V substitutions; the E93K, K115I, A211E, D226S, I421A, A444G, A531D, and F560L substitutions; or the K115I, D226S, L366M, I421A, A444G, A531D, and F560V substitutions.

In certain embodiments, the recombinant mutant isomaltase comprises the R132K, E498D, and A531D substitutions (relative to SEQ ID NO: 1). For example, the recombinant mutant isomaltase may comprise SEQ ID NO: 3, also referred to as V131 herein.

SEQ ID NO: 3
MIYTPKWWWQNSVVYQVYPRSFQDSNHDGIGDLKGIISRLDYIKKLGAD
IIWLNPIYRSPNVDNGYDISDYRAIDPTFGSLTDFKELLTKAHELGLKI
MMDLVVNHSSDENEWFKQSRQGKENPYRDYYIWKDPVDGHEPTNWGSYF
SGSAWQYDETSGQYYLHLFAVKQPDLNWENEAVRHSVYDIMNWWADLGV
DGFRMDVINLISKPAVYKDVPTAPGMQYGDVEPVVANGHRMHEFLQEMH
QAVMAKHDLVTVGETPGATTDDAKKYANLEQTELNMVFEFEHVGLDGND
NPALGKWSDKKVSLPELRDNLVKWQTQLNGKAWNSLYWNNHDQPRVVSR
FGNDDPKYRVVSAKMLATMLHCLQGTPYIYAGEELGMINTTFNSLSDYR
DLESINAYHQLVDEEHLVDGKTMSRYLAIHSRDNARTPMQWDDSKNAGF
SDAEPWIAVNPNYSEINAKAALADPSSVFYHYQKLIQMRHDLPVMTEGK
FALVNGNDLDEQVFAYTRDDGETTLLVVANFTKETIKREYDAGQGKLLL
SNYEDDMGETLRPYEAKVYEFSSKR.

In certain embodiments, the recombinant mutant isomaltase comprises the K115I, R132K, A444G, and A531D substitutions relative to SEQ ID NO: 1. For example, the recombinant mutant isomaltase may comprise SEQ ID NO: 4, also referred to as V203 herein.

SEQ ID NO: 4
MIYTPKWWWQNSVVYQVYPRSFQDSNHDGIGDLKGIISRLDYIKKLGAD
IIWLNPIYRSPNVDNGYDISDYRAIDPTFGSLTDFKELLTKAHELGLKI
MMDLVVNHSSDENEWFIQSRQGKENPYRDYYIWKDPVDGHEPTNWGSYF
SGSAWQYDETSGQYYLHLFAVKQPDLNWENEAVRHSVYDIMNWWADLGV
DGFRMDVINLISKPAVYKDVPTAPGMQYGDVEPVVANGHRMHEFLQEMH
QAVMAKHDLVTVGETPGATTDDAKKYANLEQTELNMVFEFEHVGLDGND
NPALGKWSDKKVSLPELRDNLVKWQTQLNGKAWNSLYWNNHDQPRVVSR
FGNDDPKYRVVSAKMLATMLHCLQGTPYIYAGEELGMTNTTFNSLSDYR
DLESINAYHQLVDEEHLVDGKTMSRYLAIHSRDNARTPMQWDDSKNAGF
SDGEPWIAVNPNYSEINAKAALADPSSVFYHYQKLIQMRHDLPVMTEGK
FALVNGNELDEQVFAYTRDDGETTLLVVANFTKETIKREYDAGQGKLLL
SNYEDDMGETLRPYEAKVYEFSSKR.

In certain embodiments, the recombinant mutant isomaltase comprises the K115I, R132K, D226S, I421A, A444G, A531D, and F560L substitutions relative to SEQ ID NO: 1. For example, the recombinant mutant isomaltase may comprise SEQ ID NO: 5, also referred to as V353 herein.

SEQ ID NO: 5
MIYTPKWWWQNSVVYQVYPRSFQDSNHDGIGDLKGIISRLDYIKKLGAD
IIWLNPIYRSPNVDNGYDISDYRAIDPTFGSLTDFKELLTKAHELGLKI
MMDLVVNHSSDENEWFIQSRQGKENPYRDYYIWKDPVDGHEPTNWGSYF
SGSAWQYDETSGQYYLHLFAVKQPDLNWENEAVRHSVYDIMNWWADLGV
DGFRMDVINLISKPAVYKDVPTAPGMQYGSVEPVVANGHRMHEFLQEMH
QAVMAKHDLVTVGETPGATTDDAKKYANLEQTELNMVFEFEHVGLDGND
NPALGKWSDKKVSLPELRDNLVKWQTQLNGKAWNSLYWNNHDQPRVVSR
FGNDDPKYRVVSAKMLATMLHCLQGTPYIYAGEELGMTNTTFNSLSDYR
DLESINAYHQLVDEEHLVDGKTMSRYLAAHSRDNARTPMQWDDSKNAGF
SDGEPWIAVNPNYSEINAKAALADPSSVFYHYQKLIQMRHDLPVMTEGK
FALVNGNELDEQVFAYTRDDGETTLLVVANFTKETIKREYDAGQGKLLL
SNYEDDMGETLRPYEAKVYELSSKR.

In certain embodiments, the recombinant mutant isomaltase comprises the T82A, K115I, R132K, D226S, L394I, A444G, and A53 iD substitutions relative to SEQ ID NO: 1. For example, the recombinant mutant isomaltase may comprise SEQ ID NO: 6, also referred to as V372 herein.

SEQ ID NO: 6
MIYTPKWWWQNSVVYQVYPRSFQDSNHDGIGDLKGIISRLDYIKKLGAD
IIWLNPIYRSPNVDNGYDISDYRAIDPTFGSLADFKELLTKAHELGLKI
MMDLVVNHSSDENEWFIQSRQGKENPYRDYYIWKDPVDGHEPTNWGSYF
SGSAWQYDETSGQYYLHLFAVKQPDLNWENEAVRHSVYDIMNWWADLGV
DGFRMDVINLISKPAVYKDVPTAPGMQYGSVEPVVANGHRMHEFLQEMH
QAVMAKHDLVTVGETPGATTDDAKKYANLEQTELNMVFEFEHVGLDGND
NPALGKWSDKKVSLPELRDNLVKWQTQLNGKAWNSLYWNNHDQPRVVSR
FGNDDPKYRVVSAKMLATMLHCLQGTPYIYAGEELGMTNTTFNSLSDYR
DIESINAYHQLVDEEHLVDGKTMSRYLAIHSRDNARTPMQWDDSKNAGF
SDGEPWIAVNPNYSEINAKAALADPSSVFYHYQKLIQMRHDLPVMTEGK
FALVNGNELDEQVFAYTRDDGETTLLVVANFTKETIKREYDAGQGKLLL
SNYEDDMGETLRPYEAKVYEFSSKR.

In certain embodiments, the recombinant mutant isomaltase comprises the K115I, R132K, D226S, E310A, L366M, I421A, A444G, A531D, and F560V substitutions relative to SEQ ID NO: 1. For example, the recombinant mutant isomaltase may comprise SEQ ID NO: 7, also referred to as V408 herein.

SEQ ID NO: 7
MIYTPKWWWQNSVVYQVYPRSFQDSNHDGIGDLKGIISRLDYIKKLGAD
IIWLNPIYRSPNVDNGYDISDYRAIDPTFGSLTDFKELLTKAHELGLKI
MMDLVVNHSSDENEWFIQSRQGKENPYRDYYIWKDPVDGHEPTNWGSYF
SGSAWQYDETSGQYYLHLFAVKQPDLNWENEAVRHSVYDIMNWWADLGV
DGFRMDVINLISKPAVYKDVPTAPGMQYGSVEPVVANGHRMHEFLQEMH
QAVMAKHDLVTVGETPGATTDDAKKYANLEQTELNMVFEFEHVGLDGND
NPALGKWSDKKVSLPALRDNLVKWQTQLNGKAWNSLYWNNHDQPRVVSR
FGNDDPKYRVVSAKMLATMLHCMQGTPYIYAGEELGMINTTFNSLSDYR
DLESINAYHQLVDEEHLVDGKTMSRYLAAHSRDNARTPMQWDDSKNAGF
SDGEPWIAVNPNYSEINAKAALADPSSVFYHYQKLIQMRHDLPVMTEGK
FALVNGNELDEQVFAYTRDDGETTLLVVANFTKETIKREYDAGQGKLLL
SNYEDDMGETLRPYEAKVYEVSSKR.

In certain embodiments, the recombinant mutant isomaltase comprises the substitutions T157A, E275D, and L366M relative to SEQ ID NO: 1. For example, the recombinant mutant isomaltase may comprise SEQ ID NO: 8, also referred to as V107 herein.

SEQ ID NO: 8
MIYTPKWWWQNSVVYQVYPRSFQDSNHDGIGDLKGIISRLDYIKKLGAD
IIWLNPIYRSPNVDNGYDISDYRAIDPTFGSLTDFKELLTKAHELGLKI
MMDLVVNHSSDENEWFKQSRQGKENPYRDYYIWRDPVDGHEPTNWGSYF
SGSAWQYDEASGQYYLHLFAVKQPDLNWENEAVRHSVYDIMNWWADLGV
DGFRMDVINLISKPAVYKDVPTAPGMQYGDVEPVVANGHRMHEFLQEMH
QAVMAKHDLVTVGETPGATTDDAKKYANLDQTELNMVFEFEHVGLDGND
NPALGKWSDKKVSLPELRDNLVKWQTQLNGKAWNSLYWNNHDQPRVVSR
FGNDDPKYRVVSAKMLATMLHCMQGTPYIYAGEELGMTNTTFNSLSDYR
DLESINAYHQLVDEEHLVDGKTMSRYLAIHSRDNARTPMQWDDSKNAGE
SDAEPWIAVNPNYSEINAKAALADPSSVFYHYQKLIQMRHDLPVMTEGK
FALVNGNELDEQVFAYTRDDGETTLLVVANFTKETIKREYAAGQGKLLL
SNYEDDMGETLRPYEAKVYEFSSKR.

In certain embodiments, the recombinant mutant isomaltase comprises the substitutions G533E, E543D, and G547S relative to SEQ ID NO: 1. For example, the recombinant mutant isomaltase may comprise SEQ ID NO: 9, also referred to as V177 herein.

SEQ ID NO: 9
MIYTPKWWWQNSVVYQVYPRSFQDSNHDGIGDLKGIISRLDYIKKLGAD
IIWLNPIYRSPNVDNGYDISDYRAIDPTFGSLTDFKELLTKAHELGLKI
MMDLVVNHSSDENEWFKQSRQGKENPYRDYYIWRDPVDGHEPTNWGSYF
SGSAWQYDETSGQYYLHLFAVKQPDLNWENEAVRHSVYDIMNWWADLGV
DGFRMDVINLISKPAVYKDVPTAPGMQYGDVEPVVANGHRMHEFLQEMH
QAVMAKHDLVTVGETPGATTDDAKKYANLEQTELNMVFEFEHVGLDGND
NPALGKWSDKKVSLPELRDNLVKWQTQLNGKAWNSLYWNNHDQPRVVSR
FGNDDPKYRVVSAKMLATMLHCLQGTPYIYAGEELGMTNTTFNSLSDYR
DLESINAYHQLVDEEHLVDGKTMSRYLAIHSRDNARTPMQWDDSKNAGF
SDAEPWIAVNPNYSEINAKAALADPSSVFYHYQKLIQMRHDLPVMTEGK
FALVNGNELDEQVFAYTRDDGETTLLVVANFTKETIKREYAAEQGKLLL
SNYDDDMSETLRPYEAKVYEFSSKR.

In certain embodiments, the recombinant mutant isomaltase comprises the substitutions R132K, E498D, A531D, T89A, E178P, and F560L relative to SEQ ID NO: 1. For example, the recombinant mutant isomaltase may comprise SEQ ID NO: 10, also referred to as V202 herein.

SEQ ID NO: 10
MIYTPKWWWQNSVVYQVYPRSFQDSNHDGIGDLKGIISRLDYIKKLGAD
IIWLNPIYRSPNVDNGYDISDYRAIDPTFGSLTDFKELLAKAHELGLKI
MMDLVVNHSSDENEWFKQSRQGKENPYRDYYIWKDPVDGHEPTNWGSYF
SGSAWQYDETSGQYYLHLFAVKQPDLNWENPAVRHSVYDIMNWWADLGV
DGFRMDVINLISKPAVYKDVPTAPGMQYGDVEPVVANGHRMHEFLQEMH
QAVMAKHDLVTVGETPGATTDDAKKYANLEQTELNMVFEFEHVGLDGND
NPALGKWSDKKVSLPELRDNLVKWQTQLNGKAWNSLYWNNHDQPRVVSR
FGNDDPKYRVVSAKMLATMLHCLQGTPYIYAGEELGMTNTTFNSLSDYR
DLESINAYHQLVDEEHLVDGKTMSRYLAIHSRDNARTPMQWDDSKNAGF
SDAEPWIAVNPNYSEINAKAALADPSSVFYHYQKLIQMRHDLPVMTEGK
FALVNGNDLDEQVFAYTRDDGETTLLVVANFTKETIKREYDAGQGKLLL
SNYEDDMGETLRPYEAKVYELSSKR.

In certain embodiments, the recombinant mutant isomaltase comprises the substitutions R132K, S455A, E498D, A531D, K115I, and D186E relative to SEQ ID NO: 1. For example, the recombinant mutant isomaltase may comprise SEQ ID NO: 11, also referred to as V210 herein.

SEQ ID NO: 11
MIYTPKWWWQNSVVYQVYPRSFQDSNHDGIGDLKGIISRLDYIKKLGAD
IIWLNPIYRSPNVDNGYDISDYRAIDPTFGSLTDFKELLTKAHELGLKI
MMDLVVNHSSDENEWFIQSRQGKENPYRDYYIWKDPVDGHEPTNWGSYF
SGSAWQYDETSGQYYLHLFAVKQPDLNWENEAVRHSVYEIMNWWADLGV
DGFRMDVINLISKPAVYKDVPTAPGMQYGDVEPVVANGHRMHEFLQEMH
QAVMAKHDLVTVGETPGATTDDAKKYANLEQTELNMVFEFEHVGLDGND
NPALGKWSDKKVSLPELRDNLVKWQTQLNGKAWNSLYWNNHDQPRVVSR
FGNDDPKYRVVSAKMLATMLHCLQGTPYIYAGEELGMTNTTFNSLSDYR
DLESINAYHQLVDEEHLVDGKTMSRYLAIHSRDNARTPMQWDDSKNAGF
SDAEPWIAVNPNYAEINAKAALADPSSVFYHYQKLIQMRHDLPVMTEGK
FALVNGNDLDEQVFAYTRDDGETTLLVVANFTKETIKREYDAGQGKLLL
SNYEDDMGETLRPYEAKVYEFSSKR.

In certain embodiments, the recombinant mutant isomaltase comprises the substitutions R132K, E498D, A531D, T82A, E178P, and E501D relative to SEQ ID NO: 1. For example, the recombinant mutant isomaltase may comprise SEQ ID NO: 12, also referred to as V237 herein.

SEQ ID NO: 12
MIYTPKWWWQNSVVYQVYPRSFQDSNHDGIGDLKGIISRLDYIKKLGAD
IIWLNPIYRSPNVDNGYDISDYRAIDPTFGSLADFKELLTKAHELGLKI
MMDLVVNHSSDENEWFKQSRQGKENPYRDYYIWKDPVDGHEPTNWGSYF
SGSAWQYDETSGQYYLHLFAVKQPDLNWENPAVRHSVYDIMNWWADLGV
DGFRMDVINLISKPAVYKDVPTAPGMQYGDVEPVVANGHRMHEFLQEMH
QAVMAKHDLVTVGETPGATTDDAKKYANLEQTELNMVFEFEHVGLDGND
NPALGKWSDKKVSLPELRDNLVKWQTQLNGKAWNSLYWNNHDQPRVVSR
FGNDDPKYRVVSAKMLATMLHCLQGTPYIYAGEELGMTNTTFNSLSDYR
DLESINAYHQLVDEEHLVDGKTMSRYLAIHSRDNARTPMQWDDSKNAGF
SDAEPWIAVNPNYSEINAKAALADPSSVFYHYQKLIQMRHDLPVMTEGK
FALVNGNDLDDQVFAYTRDDGETTLLVVANFTKETIKREYDAGQGKLLL
SNYEDDMGETLRPYEAKVYEFSSKR.

In certain embodiments, the recombinant mutant isomaltase comprises the amino acid sequence of any one of SEQ ID NOs: 3-12, or an amino acid sequence that has at least 85%, 90%, 91%, 92%, 93%, 94%, 95%, 96%, 97%, 98%, or 99% sequence identity to any one of SEQ ID NOs: 3-12.

In certain embodiments, the recombinant mutant isomaltase comprises the amino acid sequence of SEQ ID NO: 3, or an amino acid sequence that has at least 85%, 90%, 91%, 92%, 93%, 94%, 95%, 96%, 97%, 98%, or 99% sequence identity to SEQ ID NO: 3. In certain embodiments, the recombinant mutant isomaltase comprises the amino acid sequence of SEQ ID NO: 4, or an amino acid sequence that has at least 85%, 90%, 91%, 92%, 93%, 94%, 95%, 96%, 97%, 98%, or 99% sequence identity to SEQ ID NO: 4. In certain embodiments, the recombinant mutant isomaltase comprises the amino acid sequence of SEQ ID NO: 5, or an amino acid sequence that has at least 85%, 90%, 91%, 92%, 93%, 94%, 95%, 96%, 97%, 98%, or 99% sequence identity to SEQ ID NO: 5. In certain embodiments, the recombinant mutant isomaltase comprises the amino acid sequence of SEQ ID NO: 6, or an amino acid sequence that has at least 85%, 90%, 91%, 92%, 93%, 94%, 95%, 96%, 97%, 98%, or 99% sequence identity to SEQ ID NO: 6. In certain embodiments, the recombinant mutant isomaltase comprises the amino acid sequence of SEQ ID NO: 7, or an amino acid sequence that has at least 85%, 90%, 91%, 92%, 93%, 94%, 95%, 96%, 97%, 98%, or 99% sequence identity to SEQ ID NO: 7. In certain embodiments, the recombinant mutant isomaltase comprises the amino acid sequence of SEQ ID NO: 8, or an amino acid sequence that has at least 85%, 90%, 91%, 92%, 93%, 94%, 95%, 96%, 97%, 98%, or 99% sequence identity to SEQ ID NO: 8. In certain embodiments, the recombinant mutant isomaltase comprises the amino acid sequence of SEQ ID NO: 9, or an amino acid sequence that has at least 85%, 90%, 91%, 92%, 93%, 94%, 95%, 96%, 97%, 98%, or 99% sequence identity to SEQ ID NO: 9. In certain embodiments, the recombinant mutant isomaltase comprises the amino acid sequence of SEQ ID NO: 10, or an amino acid sequence that has at least 85%, 90%, 91%, 92%, 93%, 94%, 95%, 96%, 97%, 98%, or 99% sequence identity to SEQ ID NO: 10. In certain embodiments, the recombinant mutant isomaltase comprises the amino acid sequence of SEQ ID NO: 11, or an amino acid sequence that has at least 85%, 90%, 91%, 92%, 93%, 94%, 95%, 96%, 97%, 98%, or 99% sequence identity to SEQ ID NO: 11. In certain embodiments, the recombinant mutant isomaltase comprises the amino acid sequence of SEQ ID NO: 12, or an amino acid sequence that has at least 85%, 90%, 91%, 92%, 93%, 94%, 95%, 96%, 97%, 98%, or 99% sequence identity to SEQ ID NO: 12.

In certain embodiments, the isomaltase enzyme variant comprises at least one amino acid substitution (e.g., 1, 2, 3, 4, 5, 6, 7, 8, 9, 10 amino acid substitutions) and an amino acid sequence that has at least 98%, 98.5%, 99% or 99.5% sequence identity to SEQ ID NO: 1.

As used herein, percent “identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Similarly, percent “identity” between a nucleic acid sequence and a reference sequence is defined as the percentage of nucleotides in the nucleic acid sequence that are identical to the nucleotides in the reference sequence, after aligning the sequences and introducing gaps, if necessary, to achieve the maximum percent sequence identity. Alignment for purposes of determining percent sequence identity (e.g., amino acid sequence identity or nucleic acid sequence identity) can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402) are tailored for sequence similarity searching. For a discussion of basic issues in searching sequence databases, see Altschul et al., (1994) NATURE GENETICS 6:119-129. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter can be used at the default settings. The default scoring matrix used by blastp, blastx, tblastn, and tblastx is the BLOSUM62 matrix (Henikoff et al., (1992) PROC. NATL. ACAD. SCI. USA 89:10915-10919). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; —W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn; and -Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

In certain embodiments, a recombinant mutant isomaltase has increased stability at acidic pH (e.g., pH 4.0 or pH 6.0) relative to a corresponding wild-type isomaltase enzyme. An increased stability at acidic pH may, in certain conditions, allow the recombinant mutant isomaltase to survive acidic conditions of the digestive system, especially the stomach. An increased stability at acidic pH may, in certain conditions, also allow the recombinant mutant isomaltase to survive in certain food or beverages, e.g., fruit juice.

In certain embodiments, the isomaltase has a specific activity at about pH 4.0 of at least 25, 30, 35, 40, 45, 50, 55, 60, 65, 70, 75, or 80 μmol isomaltose consumed per minute per milligram of isomaltase. In certain embodiments, the isomaltase has at least 1 fold, 1.5 fold, 2 fold, 2.5 fold, or 3 fold higher activity at about pH 4.0, compared to a corresponding wild-type isomaltase. In certain embodiments, the isomaltase has a specific activity at about pH 6.0 of at least 30, 35, 40, 45, 50, 55, 60, 65, 70, 75, 80, 85, 90, 95, 100, 105, 110, or 115 μmol isomaltose consumed per minute per milligram of isomaltase. In certain embodiments, the isomaltase has at least 1 fold, 1.5 fold, 2 fold, 2.5 fold, or 3 fold higher activity at about pH 6.0, compared to a corresponding wild-type isomaltase. In certain embodiments, the isomaltase has a specific activity at about pH 7.0 or 7.1 of at least 25, 30, 35, 40, 45, 50, 55, 60, 65, 70, 75, 80, 85, 90, or 95 μmol isomaltose consumed per minute per milligram of isomaltase. In certain embodiments, the isomaltase has at least 1 fold, 1.5 fold, 2 fold, 2.5 fold, or 3 fold higher activity at about pH 7.0 or 7.1, compared to a corresponding wild-type isomaltase. In certain embodiments, the isomaltase retains at least 20%, 30%, 40%, 50%, 60%, 70%, 80%, 90%, 95%, 96%, 97%, 98%, or 99% of activity following incubation at about pH 3.8 for about 30 minutes. In certain embodiments, the isomaltase has at least 0.5 fold, 1 fold, 1.5 fold, 2 fold, 2.5 fold, 3 fold, 3.5 fold, 4 fold, or 5 fold higher stability at about pH 3.8 compared to a corresponding wild-type isomaltase.

Methods for testing the stability and activity of an isomaltase are known in the art and can include, for example, the methods described in Examples 1B and 1C herein. In certain embodiments, stability of an isomaltase in low pH is determined using by exposing the isomaltase to a specific pH, and using the p-nitrophenyl-alpha-glucopyranoside (pNPG method) colorimetric method to monitor glucose formation upon hydrolysis of isomaltose.

In certain embodiments, a recombinant mutant isomaltase has increased stability at higher temperature (e.g., Tm of 56-68° C.) relative to a corresponding wild-type isomaltase enzyme. In certain embodiments, the isomaltase has a Tm of at least 50, 51, 52, 53, 54, 55, 56° C. In certain embodiments the isomaltase has a Tm that is at least 1, 2, 3, 4, 5, 6, 7, 8, 9, or 10° C. higher than a corresponding wild-type isomaltase.

Methods for testing the thermostability and activity of an isomaltase are known in the art and can include, for example, the methods described in Example 1C herein. In certain embodiments, stability and activity of an isomaltase at high temperature is determined by treatment of isomaltase samples at high temperature prior to assaying residual activity using the pNPG method as described above.

In certain embodiments, the isomaltase has increased stability in the presence of a protease (e.g., a serine protease and/or an aspartic protease) relative to the corresponding wild-type isomaltase enzyme. Increased stability in the presence of a protease may, in certain conditions, allow a recombinant mutant isomaltase to survive the conditions of the stomach. In certain embodiments, a recombinant mutant isomaltase has increased stability in the presence of pancreatin or pepsin relative to a corresponding wild-type isomaltase enzyme.

In certain embodiments, a disclosed isomaltase enzyme retains at least 20%, 30%, 40%, 50%, 60%, 70%, 80%, 90%, 95%, 96%, 97%, 98%, or 99% of activity following incubation with pepsin (e.g., incubation with about 10 μg/mL pepsin at about at 37° C. in simulated gastric fluid (SGF) for about 2 hours). In certain embodiments, a disclosed isomaltase enzyme retains at least 20%, 30%, 40%, 50%, 60%, 70%, 80%, 90%, 95%, 96%, 97%, 98%, or 99% of activity following incubation with pancreatin (e.g., incubation with about 3.4 mg/ml pancreatin at about at 37° C. in simulated intestinal fluid (SGF) for about 2 hours).

Methods for testing the proteolytic stability and activity of an isomaltase are known in the art and can include, for example, the methods described in Example 1C herein. In certain embodiments, stability and activity of an isomaltase in the presence of pepsin or pancreatin is determined by incubation of isomaltase with pepsin or pancreatin prior to assaying residual activity using the pNPG method as described above.

### II. Sucrase (Invertase) Enzymes

Among other things, the invention provides pharmaceutical compositions optionally further comprising an invertase enzyme that, for example, are useful in treating disorders such as congenital sucrase-isomaltase deficiency (CSID).

As used herein, the terms “sucrase” and “invertase” are used interchangeably and refer to any enzyme, or a functional fragment thereof, that is capable of catalyzing the hydrolysis of sucrose to fructose and glucose. Sucrases and invertases are also called, acid invertases, alkaline invertases, β-D-fructofuranosidases, β-fructosidases, β-fructofuranosidases, β-fructofuranoside fructohydrolases, β-fructopyranosidases, β-h-fructosidases, β-invertases, EC 3.2.1.26, exo-β-(2,6)-fructofuraosidases, fructosylinvertases, invertins, glucosucrases, saccharases, and sucrose hydrolases, and, unless indicated otherwise, the terms are used interchangeably herein. The term invertase includes variants having one or more amino acid substitutions, deletions, or insertions relative to a wild-type invertase sequence, and/or fusion proteins or conjugates including an invertase.

As used herein, the term “functional fragment” of an invertase refers to fragment of a full-length invertase that retains, for example, at least 10%, at least 20%, at least 30%, at least 40%, at least 50%, at least 60%, at least 70%, at least 80%, at least 90%, or 100% of the enzymatic activity of the corresponding full-length, naturally occurring invertase. Invertase enzymatic activity may be assayed by any method known in the art. Exemplary invertase activity assays are described in Bacon (1955) METHODS IN ENZYMOLOGY 1: 258-262, Lever (1972) ANALYTICAL BIOCHEM. 47: 273-279, United States Patent Application Publication No. 20140250942, and on the world wide web at sigmaaldrich.com/technical-documents/protocols/biology/enzymatic-assay-of-invertase.html.

Exemplary invertase enzymes include invertase enzymes derived from Saccharomyces cerevisiae. The amino acid sequence of an exemplary wild-type invertase enzyme derived from Saccharomyces cerevisiae (including a native signal sequence) is depicted in SEQ ID NO: 13, and a nucleotide sequence encoding an exemplary wild-type invertase enzyme derived from Saccharomyces cerevisiae is depicted in SEQ ID NO: 15.

SEQ ID NO: 13 represents wild-type S. cerevisiae invertase with a
native signal sequence where the signal sequence is underlined, and
the mature protein sequence is italicized:
MLLQAFLFLLAGFAAKISASMTNETSDRPLVHFTPNKGWMNDPNGLWYDEKDAKWHLYFQYN
PNDTVWGTPLFWGHATSDDLTHWEDEPIAIAPKRNDSGAFSGSMVVDYNNTSGFFNDTIDPR
QRCVAIWTYNTPESEEQYISYSLDGGYTFTEYQKNPVLAANSTQFRDPKVFWYEPSQKWIMT
AAKSQDYKIEIYSSDDLKSWKLESAFANEGFLGYQYECPGLIEVPTEQDPSKSYWVMFISIN
PGAPAGGSFNQYFVGSFNGTHFEAFDNQSRVVDFGKDYYALQTFFNTDPTYGSALGIAWASN
WEYSAFVPTNPWRSSMSLVRKFSLNTEYQANPETELINLKAEPILNISNAGPWSRFATNTTL
TKANSYNVDLSNSTGTLEFELVYAVNTTQTISKSVFPDLSLWFKGLEDPEEYLRMGFEASAS
SFFLDRGNSKVKFVKENPYFTNRMSVNNQPFKSENDLSYYKVYGLLDQNILELYFNDGDVVS
TNTYFMTTGNALGSVNMTTGVDNLFYIDKFQVREVK.
SEQ ID NO: 16 represents wild-type S. cerevisiae invertase with
F172Y, N429A, and P479A mutations and a FAKS signal sequence (shown
as underlined):
MRFPSIFTAVLFAASSALAAPVNTTTEDETAQIPAEAVIGYSDLEGDFDVAVLPFSNSTNNG
LLFINTTIASIAAKEEGVSLEKREAEASMTNETSDRPLVHFTPNKGWMNDPNGLWYDEKDAK
WHLYFQYNPNDTVWGTPLFWGHATSDDLTHWEDEPIAIAPKRNDSGAYSGSMVVDYNNTSGF
FNDTIDPRQRCVAIWTYNTPESEEQYISYSLDGGYTFTEYQKNPVLAANSTQFRDPKVFWYE
PSQKWIMTAAKSQDYKIEIYSSDDLKSWKLESAFANEGFLGYQYECPGLIEVPTEQDPSKSY
WVMFISINPGAPAGGSFNQYFVGSFNGTHFEAFDNQSRVVDFGKDYYALQTFFNTDPTYGSA
LGIAWASNWEYSAFVPTNPWRSSMSLVRKFSLNTEYQANPETELINLKAEPILNISAAGPWS
RFATNTTLTKANSYNVDLSNSTGTLEFELVYAVNTTQTISKSVFADLSLWEKGLEDPEEYLR
MGFEASASSFFLDRGNSKVKFVKENPYFTNRMSVNNQPFKSENDLSYYKVYGLLDQNILELY
FNDGDVVSTNTYFMTTGNALGSVNMTTGVDNLFYIDKFQVREVK.
SEQ ID NO: 17 represents wild type S. cerevisiae invertase (without
a signal sequence) with mutations corresponding to F172Y, N429A,
and P479A using the numbering scheme for SEQ ID NO: 16:
SMTNETSDRPLVHFTPNKGWMNDPNGLWYDEKDAKWHLYFQYNPNDTVWGTPLFWGHATSDD
LTHWEDEPIAIAPKRNDSGAYSGSMVVDYNNTSGFFNDTIDPRQRCVAIWTYNTPESEEQYI
SYSLDGGYTFTEYQKNPVLAANSTQFRDPKVFWYEPSQKWIMTAAKSQDYKIEIYSSDDLKS
WKLESAFANEGFLGYQYECPGLIEVPTEQDPSKSYWVMFISINPGAPAGGSFNQYFVGSFNG
THFEAFDNQSRVVDFGKDYYALQTFFNTDPTYGSALGIAWASNWEYSAFVPTNPWRSSMSLV
RKFSLNTEYQANPETELINLKAEPILNISAAGPWSRFATNTTLTKANSYNVDLSNSTGTLEF
ELVYAVNTTQTISKSVFADLSLWFKGLEDPEEYLRMGFEASASSFFLDRGNSKVKFVKENPY
FTNRMSVNNQPFKSENDLSYYKVYGLLDQNILELYFNDGDVVSTNTYFMTTGNALGSVNMTT
GVDNLFYIDKFQVREVK.
SEQ ID NO: 19 represents Sc_S288C invertase (with the native signal
sequence underlined) with mutations corresponding to F172Y and N429A
using the numbering scheme for SEQ ID NO: 16:
MLLQAFLFLLAGFAAKISASMTNETSDRPLVHFTPNKGWMNDPNGLWYDEKDAKWHLYFQYN
PNDTVWGTPLFWGHATSDDLTNWEDQPIAIAPKRNDSGAYSGSMVVDYNNTSGFFNDTIDPR
QRCVAIWTYNTPESEEQYISYSLDGGYTFTEYQKNPVLAANSTQFRDPKVFWYEPSQKWIMT
AAKSQDYKIEIYSSDDLKSWKLESAFANEGFLGYQYECPGLIEVPTEQDPSKSYWVMFISIN
PGAPAGGSFNQYFVGSFNGTHFEAFDNQSRVVDFGKDYYALQTFFNTDPTYGSALGIAWASN
WEYSAFVPTNPWRSSMSLVRKFSLNTEYQANPETELINLKAEPILNISAAGPWSRFATNTTL
TKANSYNVDLSNSTGTLEFELVYAVNTTQTISKSVFADLSLWFKGLEDPEEYLRMGFEVSAS
SFFLDRGNSKVKFVKENPYFTNRMSVNNQPFKSENDLSYYKVYGLLDQNILELYFNDGDVVS
TNTYFMTTGNALGSVNMTTGVDNLFYIDKFQVREVK.
SEQ ID NO: 20 represents Sc_S288C invertase (without a signal
sequence) with mutations corresponding to F172Y and N429A using the
numbering scheme of SEQ ID NO: 16:
SMTNETSDRPLVHFTPNKGWMNDPNGLWYDEKDAKWHLYFQYNPNDTVWGTPLFWGHATSDD
LTNWEDQPIAIAPKRNDSGAYSGSMVVDYNNTSGFFNDTIDPRQRCVAIWTYNTPESEEQYI
SYSLDGGYTFTEYQKNPVLAANSTQFRDPKVEWYEPSQKWIMTAAKSQDYKIEIYSSDDLKS
WKLESAFANEGFLGYQYECPGLIEVPTEQDPSKSYWVMFISINPGAPAGGSFNQYFVGSFNG
THFEAFDNQSRVVDFGKDYYALQTFFNTDPTYGSALGIAWASNWEYSAFVPTNPWRSSMSLV
RKFSLNTEYQANPETELINLKAEPILNISAAGPWSRFATNTTLTKANSYNVDLSNSTGTLEF
ELVYAVNTTQTISKSVFADLSLWFKGLEDPEEYLRMGFEVSASSFFLDRGNSKVKFVKENPY
FTNRMSVNNQPFKSENDLSYYKVYGLLDQNILELYFNDGDVVSTNTYFMTTGNALGSVNMTT
GVDNLFYIDKFQVREVK.

Additional exemplary invertase enzymes can be found on the world wide web at brenda-enzymes.org/enzyme.php?ecno=3.2.1.26&onlyTable=Sequence. Additional exemplary invertase enzymes are described in United States Patent Application Publication No. 2014/0250942.

A contemplated invertase enzyme may comprise the amino acid sequence of any one of SEQ ID NOs: 13-14, or an amino acid sequence that has at least 80%, 85%, 90%, 95%, 96%, 97%, 98%, or 99% sequence identity to any one of SEQ ID NOs: 13-14. In some embodiments, the invertase comprises any one of SEQ ID NOs: 16-17, or a functional fragment or variant thereof. In some embodiments, the invertase comprises any one of SEQ ID NOs: 19-20, or a functional fragment or variant thereof, Other invertase enzymes that may be used in combination with the recombinant isomaltase enzymes described herein are described in U.S. Provisional Patent Application No. 63/388,810, filed on Jul. 13, 2022.

In certain embodiments, the invertase enzyme comprises one or more conservative substitutions relative to invertase enzyme disclosed herein. In other embodiments, the invertase enzyme comprises one or more non-conservative substitutions relative to invertase enzyme disclosed herein.

### III. Enzyme Production

Methods for producing isomaltase and/or invertase enzymes are known in the art. For example, DNA molecules encoding an isomaltase and/or invertase enzyme can be chemically synthesized using the sequence information provided herein. Synthetic DNA molecules can be ligated to other appropriate nucleotide sequences, including, e.g., expression control sequences, to produce conventional gene expression constructs encoding the desired isomaltase and/or invertase enzyme.

Nucleic acids encoding desired isomaltase and/or invertase enzymes can be incorporated (ligated) into expression vectors, which can be introduced into host cells through conventional transfection or transformation techniques. Transformed host cells can be grown under conditions that permit the host cells to express the genes that encode the isomaltase and/or invertase enzyme.

An exemplary DNA sequence encoding a wild-type L. fermentum
isomaltase is provided in SEQ ID NO: 2.
SEQ ID NO: 2 (wild type L. fermentum isomaltase DNA sequence):
ATGATATATACACCAAAATGGTGGTGGCAAAATTCAGTTGTCTACCAAGTCTATCCACGGAG
TTTTCAAGACAGCAATCATGATGGCATTGGTGATTTAAAAGGAATCATCAGTCGGCTTGACT
ATATTAAAAAACTAGGTGCTGATATTATCTGGCTCAATCCAATCTACCGTTCGCCAAACGTT
GATAATGGGTATGACATCAGTGATTATCGGGCAATTGATCCAACTTTTGGCTCATTGACCGA
CTTTAAGGAGCTGCTGACAAAGGCTCATGAACTTGGATTAAAAATAATGATGGATCTGGTGG
TCAATCACTCATCAGATGAAAATGAATGGTTTAAGCAAAGCCGCCAAGGAAAAGAAAATCCA
TACCGTGACTATTATATCTGGCGTGATCCAGTCGATGGCCATGAACCAACTAATTGGGGATC
ATACTTCTCTGGTTCAGCTTGGCAGTACGATGAGACAAGCGGTCAATACTACCTTCACCTGT
TCGCTGTAAAGCAGCCGGATCTAAACTGGGAAAATGAAGCCGTTCGTCATTCGGTATACGAC
ATCATGAATTGGTGGGCAGATCTCGGTGTCGATGGATTTCGAATGGATGTCATCAACTTAAT
TTCAAAACCGGCTGTCTACAAAGACGTTCCAACCGCGCCCGGAATGCAGTATGGCGATGTCG
AGCCGGTTGTTGCCAATGGTCATCGGATGCACGAATTCCTTCAAGAAATGCATCAAGCTGTA
ATGGCCAAGCATGATTTGGTAACAGTTGGCGAAACACCTGGTGCTACAACAGATGATGCCAA
GAAGTACGCCAACCTTGAACAGACTGAGCTGAACATGGTTTTTGAGTTTGAGCATGTCGGCT
TAGACGGTAACGACAACCCAGCTTTAGGAAAATGGAGCGACAAAAAAGTCAGTCTGCCAGAA
TTGCGAGATAACCTGGTGAAATGGCAGACTCAGCTGAATGGTAAGGCTTGGAACTCGCTTTA
CTGGAACAACCATGATCAGCCACGCGTCGTATCCCGTTTTGGCAATGATGATCCAAAATATC
GTGTCGTTTCGGCTAAAATGTTGGCAACAATGCTGCACTGTCTGCAAGGCACGCCTTATATC
TACGCAGGTGAGGAACTGGGAATGACCAACACGACCTTTAACTCTTTGTCTGACTATCGTGA
TTTGGAGAGCATTAATGCCTACCATCAGCTGGTTGACGAAGAGCATCTAGTTGATGGAAAGA
CAATGAGCAGATATCTGGCAATTCATTCTCGGGACAATGCCAGAACGCCAATGCAATGGGAT
GACAGCAAGAATGCCGGATTTTCGGATGCTGAACCTTGGATCGCGGTCAATCCCAACTATTC
AGAGATCAACGCAAAGGCGGCACTGGCGGATCCATCATCCGTCTTCTATCACTACCAGAAAC
TCATTCAAATGCGCCATGACTTGCCAGTAATGACAGAAGGAAAGTTTGCGTTGGTCAATGGC
AACGAATTGGATGAGCAGGTCTTTGCTTACACGCGTGATGATGGAGAAACAACGCTGCTTGT
AGTAGCCAACTTTACTAAGGAAACAATCAAGCGAGAATACGCGGCTGGTCAAGGCAAGCTCT
TATTAAGCAACTACGAGGATGACATGGGAGAAACTCTGCGTCCATATGAAGCTAAAGTATAT
GAATTCAGTTCAAAGAGGTAA
An exemplary DNA sequence encoding a wild-type S. cerevisiae
invertase provided in SEQ ID NO: 15.
SEQ ID NO: 15 (wild type S. cerevisiae invertase DNA sequence):
ATGCTTTTGCAAGCTTTCCTTTTCCTTTTGGCTGGTTTTGCAGCCAAAATATCTGCATCAAT
GACAAACGAAACTAGCGATAGACCTTTGGTCCACTTCACACCCAACAAGGGCTGGATGAATG
ACCCAAATGGGTTGTGGTACGATGAAAAAGATGCCAAATGGCATCTGTACTTTCAATACAAC
CCAAATGACACCGTATGGGGTACGCCATTGTTTTGGGGCCATGCTACTTCCGATGATTTGAC
TCATTGGGAAGATGAACCCATTGCTATCGCTCCCAAGCGTAACGATTCAGGTGCTTTCTCTG
GCTCCATGGTGGTTGATTACAACAACACGAGTGGGTTTTTCAATGATACTATTGATCCAAGA
CAAAGATGCGTTGCGATTTGGACTTATAACACTCCTGAAAGTGAAGAGCAATACATTAGCTA
TTCTCTTGATGGTGGTTACACTTTTACTGAATACCAAAAGAACCCTGTTTTAGCTGCCAACT
CCACTCAATTCAGAGATCCAAAGGTGTTCTGGTATGAACCTTCTCAAAAATGGATTATGACG
GCTGCCAAATCACAAGACTACAAAATTGAAATTTACTCCTCTGATGACTTGAAGTCCTGGAA
GCTAGAATCTGCATTTGCTAATGAAGGTTTCTTAGGCTACCAATATGAATGTCCAGGTTTGA
TTGAAGTCCCAACTGAGCAAGATCCTTCCAAATCCTATTGGGTCATGTTTATTTCTATCAAT
CCAGGTGCACCTGCTGGCGGTTCCTTCAACCAATATTTTGTTGGATCCTTCAATGGTACTCA
TTTTGAAGCGTTTGACAATCAATCTAGAGTGGTAGATTTTGGTAAGGACTACTATGCCTTGC
AAACTTTCTTCAACACAGACCCAACGTACGGTTCAGCATTAGGTATTGCCTGGGCTTCAAAC
TGGGAGTACAGTGCCTTTGTCCCAACTAACCCATGGAGATCATCCATGTCTTTGGTCCGCAA
GTTTTCTTTGAACACTGAATATCAAGCTAATCCAGAGACTGAATTGATCAATTTGAAAGCCG
AACCAATATTGAACATTAGTAATGCTGGTCCCTGGTCTCGTTTTGCTACTAACACAACTCTA
ACTAAGGCCAATTCTTACAATGTCGATTTGAGCAACTCGACTGGTACCCTAGAGTTTGAGTT
GGTTTACGCTGTTAACACCACACAAACCATATCCAAATCCGTCTTTCCCGACTTATCACTTT
GGTTCAAGGGTTTAGAAGATCCTGAAGAATATTTAAGAATGGGTTTTGAAGCCAGTGCTTCT
TCCTTCTTTTTGGACCGTGGTAACTCTAAGGTCAAGTTTGTCAAGGAGAACCCATATTTCAC
AAACAGAATGTCTGTCAACAACCAACCATTCAAGTCTGAGAACGACCTAAGTTACTATAAAG
TGTACGGCCTACTGGATCAAAACATCTTGGAATTGTACTTCAACGATGGAGATGTGGTTTCT
ACAAATACCTACTTCATGACCACCGGTAACGCTCTAGGATCTGTGAACATGACCACTGGTGT
CGATAATTTGTTCTACATTGACAAGTTCCAAGTAAGGGAAGTAAAATAG

Nucleic acids encoding a recombinant mutant isomaltase and/or invertase may be generated by mutating a nucleotide sequence encoding a wild type isomaltase, e.g., SEQ ID NO: 1, or a wild type invertase, e.g., SEQ ID NOs: 13 or 14, using methods known in the art. Furthermore, nucleic acids encoding such recombinant isomaltase and/or invertase enzymes may be codon optimized for expression in a heterologous cell, e.g., an Escherichia col, Saccharomyces cerevisiae, or Pichia pastoris cell, using methods known in the art.

In one embodiment, the disclosure relates to a nucleotide sequence encoding a recombinant mutant isomaltase that comprises the following substitutions: R132K, E498D, and A531D, relative to SEQ ID NO:1, e.g., a recombinant mutant isomaltase referred to as V131 herein.

In one embodiment, the disclosure relates to a nucleotide sequence encoding a recombinant mutant isomaltase that comprises the following substitutions: K115I, R132K, A444G, and A531D, relative to SEQ ID NO:1, e.g., a recombinant mutant isomaltase referred to as V203 herein.

In one embodiment, the disclosure relates to a nucleotide sequence encoding a recombinant mutant isomaltase that comprises the following substitutions: K115I, R132K, D226S, I421A, A444G, A531D, and F560L, relative to SEQ ID NO:1, e.g., a recombinant mutant isomaltase referred to as V353 herein.

In one embodiment, the disclosure relates to a nucleotide sequence encoding a recombinant mutant isomaltase that comprises the following substitutions: T82A, K115I, R132K, D226S, L394I, A444G, and A531D, relative to SEQ ID NO:1, e.g., a recombinant mutant isomaltase referred to as V372 herein.

In one embodiment, the disclosure relates to a nucleotide sequence encoding a recombinant mutant isomaltase that comprises the following substitutions: K115, R132K, D226S, E310A, L366M, I421A, A444G, A531D, F560V, relative to SEQ ID NO:1, e.g., a recombinant mutant isomaltase referred to as V408 herein.

In one embodiment, the disclosure relates to a nucleotide sequence encoding a recombinant mutant isomaltase that comprises the following substitutions: T157A, E275D, and L366M, relative to SEQ ID NO:1, e.g., a recombinant mutant isomaltase referred to as V107 herein.

In one embodiment, the disclosure relates to a nucleotide sequence encoding a recombinant mutant isomaltase that comprises the following substitutions: G533E, E543D, and G547S, relative to SEQ ID NO:1, e.g., a recombinant mutant isomaltase referred to as V177 herein.

In one embodiment, the disclosure relates to a nucleotide sequence encoding a recombinant mutant isomaltase that comprises the following substitutions: R132K, E498D, A531D, T89A, E178P, F560L, relative to SEQ ID NO:1, e.g., a recombinant mutant isomaltase referred to as V202 herein.

In one embodiment, the disclosure relates to a nucleotide sequence encoding a recombinant mutant isomaltase that comprises the following substitutions: R132K, S455A, E498D, A531D, K115I, and D186E, relative to SEQ ID NO:1, e.g., a recombinant mutant isomaltase referred to as V210 herein.

In one embodiment, the disclosure relates to a nucleotide sequence encoding a recombinant mutant isomaltase that comprises the following substitutions: R132K, E498D, A531D, T82A, E178P, E501D, relative to SEQ ID NO:1, e.g., a recombinant mutant isomaltase referred to as V237 herein.

Specific expression and purification conditions will vary depending upon the expression system employed. For example, if a gene is to be expressed in E. coli, S. cerevisiae, or P. pastoris, it can be cloned into an expression vector by positioning the engineered gene downstream from a suitable bacterial or yeast promoter, e.g., Trp or Tac, and a prokaryotic or eukaryotic signal sequence. In yeast, it may be advantageous to introduce the coding gene into the yeast genome (such as by homologous recombination) for higher expression. The expressed protein accumulates in refractile or inclusion bodies or is secreted, and can be harvested after disruption of the cells by French press or sonication, or by simply collecting the supernatant if secreted. The refractile bodies then are solubilized, and the proteins refolded and cleaved by methods known in the art. Secreted proteins can be further purified using methods known in the art such as ion exchange, affinity chromatography, or salt precipitation.

An isomaltase and/or invertase enzyme can be produced by growing (culturing) a host cell transfected with an expression vector encoding such isomaltase and/or invertase enzyme, under conditions that permit expression of the isomaltase and/or invertase enzyme. Following expression, the isomaltase and/or invertase enzyme can be harvested and purified or isolated using techniques known in the art, e.g., affinity tags such as glutathione-S-transferase (GST) and histidine tags.

Exemplary methods for recombinant expression and purification of isomaltase enzymes are described in Deng et al. (2014) FEBS OPEN BIO 4:200-212. Exemplary methods for recombinant expression and purification of invertase enzymes are described in Mohandesi et al. (2016) BIOTECH 6(2):129.

In certain embodiments, an isomaltase and/or invertase enzyme is dried, e.g., spray-dried or freeze-dried. Pharmaceutical proteins may be dried in many ways, e.g., by removal of water, organic solvent or liquid polymer by means including drying with N₂, air or inert gases, vacuum oven drying, lyophilization, washing with a volatile organic solvent followed by evaporation of the solvent, evaporation in a fume hood, tray drying, fluid bed drying, spray drying, vacuum drying, or roller drying.

Spray drying isomaltase and/or invertase enzymes allows water to be separated from the isomaltase and/or invertase enzyme preparation, allowing for continuous production of dry solids in powder, granulate, or agglomerate form from liquid feedstocks such as emulsions and pumpable suspensions. Spray drying involves the atomization of a liquid feedstock comprising isomaltase and/or invertase enzyme into a spray of droplets and contacting the droplets with hot air or gas in a drying chamber. The atomization process may be conducted using a two-fluid atomizer that mixes the liquid feedstock with a drying gas such as compressed air or nitrogen. Operating conditions and dryer design are selected according to the drying characteristics of the isomaltase and/or invertase enzyme and the desired powder qualities. Exemplary methods for spray drying enzymes are described in United States Patent Application Publication No. 2015/0353913. The isomaltase and/or invertase enzyme may be spray dried separately or combined and spray dried together.

It is contemplated that a disclosed isomaltase and/or invertase enzyme may be modified, engineered or chemically conjugated. For example, it is contemplated that a disclosed isomaltase and/or invertase enzyme can be conjugated to an effector agent using standard in vitro conjugation chemistries. If the effector agent is a polypeptide, the isomaltase and/or invertase enzyme can be chemically conjugated to the effector or joined to the effector as a fusion protein. Construction of fusion proteins is within ordinary skill in the art.

In certain embodiments, depending upon a particular mode of administration or site of activity, a disclosed isomaltase and/or invertase enzyme can be modified with a moiety that improves its stabilization and/or retention in circulation, e.g., in blood, serum, or other tissues. For example, a disclosed isomaltase and/or invertase enzyme may be conjugated to a polymer, e.g., a substantially non-antigenic polymer, such as a polyalkylene oxide or a polyethylene oxide. In certain embodiments, a disclosed isomaltase and/or invertase enzyme is conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof. Additional useful polymers include polyoxyalkylenes such as polyoxyethylene, polyoxypropylene, and block copolymers of polyoxyethylene and polyoxypropylene, polymethacrylates, carbomers, and branched or unbranched polysaccharides.

### IV. Pharmaceutical Compositions

For therapeutic use, an isomaltase and/or invertase enzyme described herein preferably is combined with a pharmaceutically acceptable carrier and/or an excipient. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., REMINGTON'S PHARMACEUTICAL SCIENCES, 18th Edition (Easton, Pennsylvania: Mack Publishing Company, 1990. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In another aspect, the disclosure relates to a pharmaceutical composition comprising: an isomaltase enzyme, e.g., a recombinant mutant isomaltase as described herein; a separate invertase enzyme; and a pharmaceutically acceptable carrier and/or an excipient. In certain embodiments, the isomaltase and/or the invertase is spray-dried.

In certain embodiments of the pharmaceutical compositions disclosed herein (i) the isomaltase is a microbial isomaltase (e.g., derived from L. fermentum), or a functional fragment or variant thereof, (ii) the isomaltase comprises a sequence of any one of SEQ ID NOs: 1-12, or a functional fragment or variant thereof, (iii) the isomaltase is a recombinant mutant L. fermentum isomaltase as described herein, (iv) the invertase is a microbial invertase (e.g., derived from Saccharomyces cerevisiae), or a functional fragment or variant thereof, (v) the invertase comprises any one of SEQ ID NOs: 13-14, or a functional fragment or variant thereof, (vi) the invertase comprises any one of SEQ ID NOs: 16-17 or a functional fragment or variant thereof, (vii) the invertase comprises any one of SEQ ID NOs: 19-20 or a functional fragment or variant thereof, (viii) the invertase is a recombinant mutant invertase set forth in U.S. Provisional Patent Application No. 63/388,810, filed on Jul. 13, 2022, or (ix) or a combination of any one of features (i)-(viii).

In certain embodiments, the pharmaceutical composition is formulated as an oral dosage form. The pharmaceutical composition can be formulated as a powder, satchel, granulate, pellet, micropellet, tablet, or minitablet, more particularly, a powder, satchel, or tablet. The resulting pharmaceutical composition can have a shelf-life at room temperature of at least 3 months, 6 months, 9 months, 12 months, 15 months, 18 months, 21 months, 24 months, 36 months, 48 months, 60 months, 72 months, 84 months, 96 months, 108 months, or 120 months.

In certain embodiments, the isomaltase and/or invertase enzymes can be formulated, or co-administered (either at the same time or sequentially), for example, by an enteral route (e.g., orally), with a pH increasing agent, for example, a protein pump inhibitor (PPI), to enhance the stability of the isomaltase and/or invertase enzyme, for example, in an acidic environment, for example, in the gastrointestinal tract.

Proton pump inhibitors are a group of drugs whose main action is pronounced and long-lasting reduction of gastric acid production. Proton pump inhibitors act by blocking the hydrogen/potassium adenosine triphosphatase enzyme system (the H⁺/K⁺ ATPase, or more commonly just gastric proton pump) of the gastric parietal cell. The proton pump is the terminal stage in gastric acid secretion, being directly responsible for secreting H⁺ ions into the gastric lumen, making it an ideal target for inhibiting acid secretion. Examples of proton pump inhibitors include: Omeprazole (brand names: LOSEC®, PRILOSEC®, ZEGERID®); Lansoprazole (brand names: PREVACID®, ZOTON®, INHIBITOL); Esomeprazole (brand names: NEXIUM®); and Pantoprazole (brand names: PROTONIX®, SOMAC®, PANTOLOC®).

Pharmaceutical compositions containing a recombinant isomaltase and/or invertase enzyme disclosed herein can be presented in a dosage unit form and can be prepared by any suitable method. A pharmaceutical composition should be formulated to be compatible with its intended route of administration. The pharmaceutical compositions may be in a variety of forms. These include, for example, liquid, semi-solid and solid dosage forms, such as liquid solutions, dispersions or suspensions, tablets, pills, powders, liposomes and suppositories. The preferred form will depend upon the intended mode of administration and therapeutic application. In certain embodiments, the composition is formulated as an oral dosage form. The oral dosage form may, for example, be formulated as a powder, satchel, granulate, pellet, micropellet, tablet, or minitablet. In some embodiments, the composition is formulated as a powder, satchel, or tablet.

Although the compositions preferably are formulated for administration enterally (for example, orally), the compositions can be formulated for administration by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and infrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Depending upon the mode of administration, for example, by parenteral administration, it may be desirable to produce a pharmaceutical formulation that is sterile. Sterilization can be accomplished by any suitable method, e.g., filtration through sterile filtration membranes. Where the composition is lyophilized, filter sterilization can be conducted prior to or following lyophilization and reconstitution.

In certain embodiments, a disclosed composition comprises a polyionic reagent which may, e.g., coat the isomaltase and/or invertase enzyme (e.g., the composition comprises a polyionic coating). Exemplary polyionic reagents include PSS (poly(Sodium 4-styrenesulfonate), PAA (poly Acrylic acid sodium salt), PMG (poly(methylene-co-guanidine) hydrochloride), DS (dextran sulfate), PMA (poly(methyl acrylate)), or PVS (polyvinylsiloxane).

In certain embodiments, a disclosed composition and/or dosage comprises: (i) about 750 to about 75,000, about 750 to about 60,000, about 750 to about 45,000, about 750 to about 30,000, about 750 to about 15,000, about 750 to about 10,000, about 750 to about 7,500, about 750 to about 5,000, about 750 to about 2,500, about 750 to about 1,000, about 1,000 to about 75,000, about 1,000 to about 60,000, about 1,000 to about 45,000, about 1,000 to about 30,000, about 1,000 to about 15,000, about 1,000 to about 10,000, about 1,000 to about 7,500, about 1,000 to about 5,000, about 1,000 to about 2,500, about 2,500 to about 75,000, about 2,500 to about 60,000, about 2,500 to about 45,000, about 2,500 to about 30,000, about 2,500 to about 15,000, about 2,500 to about 10,000, about 2,500 to about 7,500, about 2,500 to about 5,000, about 5,000 to about 75,000, about 5,000 to about 60,000, about 5,000 to about 45,000, about 5,000 to about 30,000, about 5,000 to about 15,000, about 5,000 to about 10,000, about 5,000 to about 7,500, about 7,500 to about 75,000, about 7,500 to about 60,000, about 7,500 to about 45,000, about 7,500 to about 30,000, about 7,500 to about 15,000, about 7,500 to about 10,000, about 10,000 to about 75,000, about 10,000 to about 60,000, about 10,000 to about 45,000, about 10,000 to about 30,000, about 10,000 to about 15,000, about 15,000 to about 75,000, about 15,000 to about 60,000, about 15,000 to about 45,000, about 15,000 to about 30,000, about 30,000 to about 75,000, about 30,000 to about 60,000, about 30,000 to about 45,000, about 45,000 to about 75,000, about 45,000 to about 60,000, or about 60,000 to about 75,000 international units (I.U.) of isomaltase enzyme; and/or (ii) about 750 to about 75,000, about 750 to about 60,000, about 750 to about 45,000, about 750 to about 30,000, about 750 to about 15,000, about 750 to about 10,000, about 750 to about 7500, about 750 to about 5,000, about 750 to about 2500, about 750 to about 1,000, about 1,000 to about 75,000, about 1,000 to about 60,000, about 1,000 to about 45,000, about 1,000 to about 30,000, about 1,000 to about 15,000, about 1,000 to about 10,000, about 1,000 to about 7,500, about 1,000 to about 5,000, about 1,000 to about 2,500, about 2,500 to about 75,000, about 2,500 to about 60,000, about 2,500 to about 45,000, about 2,500 to about 30,000, about 2,500 to about 15,000, about 2,500 to about 10,000, about 2,500 to about 7,500, about 2,500 to about 5,000, about 5,000 to about 75,000, about 5,000 to about 60,000, about 5,000 to about 45,000, about 5,000 to about 30,000, about 5,000 to about 15,000, about 5,000 to about 10,000, about 5,000 to about 7,500, about 7,500 to about 75,000, about 7,500 to about 60,000, about 7,500 to about 45,000, about 7,500 to about 30,000, about 7,500 to about 15,000, about 7,500 to about 10,000, about 10,000 to about 75,000, about 10,000 to about 60,000, about 10,000 to about 45,000, about 10,000 to about 30,000, about 10,000 to about 15,000, about 15,000 to about 75,000, about 15,000 to about 60,000, about 15,000 to about 45,000, about 15,000 to about 30,000, about 30,000 to about 75,000, about 30,000 to about 60,000, about 30,000 to about 45,000, about 45,000 to about 75,000, about 45,000 to about 60,000, or about 60,000 to about 75,000 international units (I.U.) of invertase enzyme.

In certain embodiments, a disclosed composition and/or dosage comprises: (i) about 5,000 to about 12,000, about 5,000 to about 11,000, about 5,000 to about 10,000, about 5,000 to about 9,000, about 5,000 to about 8,000, about 5,000 to about 7,000, about 5,000 to about 6,000, about 6,000 to about 12,000, about 6,000 to about 11,000, about 6,000 to about 10,000, about 6,000 to about 9,000, about 6,000 to about 8,000, about 6,000 to about 7,000, about 7,000 to about 12,000, about 7,000 to about 11,000, about 7,000 to about 10,000, about 7,000 to about 9,000, about 7,000 to about 8,000, about 8,000 to about 12,000, about 8,000 to about 11,000, about 8,000 to about 10,000, about 8,000 to about 9,000, about 9,000 to about 12,000, about 9,000 to about 11,000, about 9,000 to about 10,000, about 10,000 to about 12,000, about 10,000 to about 11,000, about 11,000 to about 12,000, about 5,000, about 6,000, about 7,000, about 8,000, about 8,500, about 9,000, about 10,000, about 11,000, or about 12,000 international units (I.U.) of isomaltase enzyme; and/or (ii) about 5,000 to about 12,000, about 5,000 to about 11,000, about 5,000 to about 10,000, about 5,000 to about 9,000, about 5,000 to about 8,000, about 5,000 to about 7,000, about 5,000 to about 6,000, about 6,000 to about 12,000, about 6,000 to about 11,000, about 6,000 to about 10,000, about 6,000 to about 9,000, about 6,000 to about 8,000, about 6,000 to about 7,000, about 7,000 to about 12,000, about 7,000 to about 11,000, about 7,000 to about 10,000, about 7,000 to about 9,000, about 7,000 to about 8,000, about 8,000 to about 12,000, about 8,000 to about 11,000, about 8,000 to about 10,000, about 8,000 to about 9,000, about 9,000 to about 12,000, about 9,000 to about 11,000, about 9,000 to about 10,000, about 10,000 to about 12,000, about 10,000 to about 11,000, about 11,000 to about 12,000, about 5,000, about 6,000, about 7,000, about 8,000, about 8,500, about 9,000, about 10,000, about 11,000, or about 12,000 international units (I.U.) of invertase enzyme.

In certain embodiments, a disclosed isomaltase and/or invertase enzyme or composition is administered to a subject together with a meal or snack. In certain embodiments, a disclosed isomaltase and/or invertase enzyme or composition is administered to a subject together with each meal or snack that the subject eats. In certain embodiments, a disclosed isomaltase and/or invertase enzyme or composition is administered to a subject once every 7 days, once every 6 days, once every 5 days, once every 4 days, once every 3 days, once every 2 days, once every day, 2 times every day, 3 times every day, 4 times every day, 5 times every day, 6 times every, or more than 6 times every day.

Depending upon the circumstances, the composition can be formulated as a powder, granules, pellet, minitablet or a micropellet. The composition can be encapsulated in a capsule, e.g., a hydroxypropyl methylcellulose (HPMC) capsule, soft gelatin capsule, or a hard gelatin capsule. Alternatively, the composition can be formulated as a tablet dosage form. The dosage form, e.g., granules, tablets, minitablets, pellets, micropellets, capsules can be enterically coated using, e.g., one or more methacrylic polymers, such as methacrylic acid methylmethacrylate copolymers (e.g., Eudragit L and S), and methacrylic acid ethyl acrylate copolymer (Eudragit L30D), hydroxyl propyl methyl cellulose acetate, hydroxy propyl methyl cellulose phthalate succinate and others.

In certain embodiments, the composition comprises less than 50%, less than 40%, less than 30%, less than 20%, less than 10% or less than 5% glycerol. In certain embodiments, the composition does not comprise glycerol.

In certain embodiments, a disclosed pharmaceutical composition has a shelf-life (e.g., retains at least 30%, at least 40%, at least 50%, at least 60%, at least 70%, at least 80%, at least 85%, at least 90%, at least 95%, at least 96%, at least 97%, at least 98%, at least 99%, or 100% of its biological activity) at room temperature of at least 3 months, 6 months, 9 months, 12 months, 15 months, 18 months, 21 months, 24 months, 36 months, 48 months, 60 months, 72 months, 84 months, 96 months, 108 months, or 120 months. In certain embodiments, a disclosed pharmaceutical composition has a shelf-life at room temperature of from about 3 to about 120 months, from about 3 to about 96 months, from about 3 to about 72 months, from about 3 to about 48 months, from about 3 to about 24 months, from about 3 to about 21 months, from about 3 to about 18 months, from about 3 to about 15 months, from about 3 to about 12 months, from about 3 to about 9 months, from about 3 to about 6 months, from about 6 to about 120 months, from about 6 to about 96 months, from about 6 to about 72 months, from about 6 to about 48 months, from about 6 to about 24 months, from about 6 to about 21 months, from about 6 to about 18 months, from about 6 to about 15 months, from about 6 to about 12 months, from about 6 to about 9 months, from about 9 to about 120 months, from about 9 to about 96 months, from about 9 to about 72 months, from about 9 to about 48 months, from about 9 to about 24 months, from about 9 to about 21 months, from about 9 to about 18 months, from about 9 to about 15 months, from about 9 to about 12 months, from about 12 to about 120 months, from about 12 to about 96 months, from about 12 to about 72 months, from about 12 to about 48 months, from about 12 to about 24 months, from about 12 to about 21 months, from about 12 to about 18 months, from about 12 to about 15 months, from about 15 to about 120 months, from about 15 to about 96 months, from about 15 to about 72 months, from about 15 to about 48 months, from about 15 to about 24 months, from about 15 to about 21 months, from about 15 to about 18 months, from about 18 to about 120 months, from about 18 to about 96 months, from about 18 to about 72 months, from about 18 to about 48 months, from about 18 to about 24 months, from about 18 to about 21 months, from about 21 to about 120 months, from about 21 to about 96 months, from about 21 to about 72 months, from about 21 to about 48 months, from about 21 to about 24 months, from about 24 to about 120 months, from about 24 to about 96 months, from about 24 to about 72 months, from about 24 to about 48 months, from about 48 to about 120 months, from about 48 to about 96 months, from about 48 to about 72 months, from about 72 to about 120 months, from about 72 to about 96 months, or from about 96 to about 120 months. In certain embodiments, a disclosed pharmaceutical composition retains at least 30%, at least 40%, at least 50%, at least 60%, at least 70%, at least 80%, at least 85%, at least 90%, at least 95%, at least 96%, at least 97%, at least 98%, at least 99%, or 100% of its biological activity after 12 hours, 1 day, 2 days, 3 days, 4 days, 5 days, 6 days, 1 week, 2 weeks, 3 weeks, or 4 weeks at 40° C. and 75% relative humidity (RH).

### V. Therapeutic Uses

The methods and compositions disclosed herein can be used to treat diseases or disorders. For example, the disclosure provides a method of treating congenital sucrase-isomaltase deficiency (CSID) in a subject. The method comprises administering (e.g., orally administering) to the subject an effective amount of (i) an isomaltase enzyme (e.g., a recombinant mutant isomaltase enzyme described herein), and/or (ii) an invertase enzyme. For example, the method may comprise administering a disclosed pharmaceutical composition comprising (i) a spray-dried isomaltase enzyme (e.g., a recombinant mutant isomaltase enzyme described herein), and/or (ii) a spray-dried invertase enzyme. In certain embodiments, the pharmaceutical composition is administered to the subject together with a meal or snack.

Under certain conditions, the method reduces abdominal pain, bloating, and/or nausea in the subject, and as a result, the method reduces a total symptom score (for example, measured by abdominal pain, bloating and nausea) for the subject. Furthermore, the invention permits greater diet liberalization in the subject (e.g., allows the subject to better tolerate certain foods, e.g., grains (e.g., wheat, potatoes, corn, rice, and bread), fruits (e.g., apples, bananas, apricots, and oranges) and/or vegetables (e.g., beets, carrots, and black beans)).

The term “effective amount” as used herein refers to the amount of an active agent (e.g., a disclosed isomaltase and/or invertase enzyme) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

The disclosure also provides a method of reducing an amount of sucrose and branched (1-6 linked) α-limit dextrin in a subject, for example, a subject with congenital sucrase-isomaltase deficiency (CSID). The method comprises administering (e.g., orally administering) to the subject an effective amount of (i) an isomaltase enzyme (e.g., a recombinant mutant isomaltase enzyme described herein), and/or (ii) an invertase enzyme. For example, the method may comprise administering a disclosed pharmaceutical composition comprising (i) a spray-dried isomaltase (e.g., a recombinant mutant isomaltase described herein), and (ii) a spray-dried invertase enzyme. An amount of sucrose and branched (1-6 linked) α-limit dextrin in a subject may refer to an amount of sucrose and branched (1-6 linked) α-limit dextrin measured by a hydrogen breath test, e.g., a sucrose methane hydrogen breath test or a ¹³C-sucrose breath test. An amount of sucrose and branched (1-6 linked) α-limit dextrin in a subject may refer to an amount of sucrose and branched (1-6 linked) α-limit dextrin in a body fluid (e.g., blood, plasma, serum, or urine), tissue and/or cell in the subject.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein, is administered in combination with one or more fructose degrading enzymes. Exemplary fructose degrading enzymes include glucose (xylose) isomerases.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1—Isomaltase Selection and Isomaltase Engineering

This Example describes the initial design of recombinant mutant L. fermentum isomaltases with improved tolerance of low pH, resistance to digestion by pepsin and pancreatin, improved thermostability, high catalytic activity at low and medium pH, and high expression in an expression host suitable for large scale production.

The goals for isomaltase engineering were to design an isomaltase enzyme with one or more of the following features:
Isomaltase activity at physiologically relevant pH conditions of the digestive tract (pH 4-7),
Thermostability at 37° C. (body temperature),
Survivability at low pH,
Survivability against pepsin in the stomach,
Survivability against pancreatin in the stomach, and
Increased expression.

Numerous different isomaltases were screened and evaluated for activity at physiologically relevant pH conditions (pH 4.0-7.0). The base (i.e., the wild-type (WT) starting) isomaltase used for mutational analysis was derived from the bacterial L. fermentum isomaltase (ID: EQC59045.1; Limosilactobacillus fermentum MTCC 8711) and comprising the amino acid sequence of SEQ ID NO: 1.

### Isomaltase Engineering

At the outset, a large number of mutant L. fermentum isomaltases were designed, each with three amino acid substitutions relative to the wild-type sequence, at least 50 different amino acid substitutions were sampled in combination. More specifically, L. fermentum isomaltase variants, each having three substitutions relative to the natural wild-type amino acid sequence, were designed, expressed in E. coli, tested, and where appropriate compared against the wild type enzyme (namely, the protein of SEQ ID NO. 1). For detailed methodology, see Liao J, et al. “Engineering proteinase K using machine learning and synthetic genes.” BMC (2007) BIOTECHNOL. 7:16. doi: 10.1186/1472-6750-7-16. PMID: 17386103; PMCID: PMC1847811.

Each of the substitutions were sampled and tested for (i) expression level (see, Example 1A), (ii) activity at pH 4 (see, Example 1B), (iii) activity at pH 7 (see, Example 1B), (iv) thermostability (see, Example 1C), (v) stability at pH 4 (see, Example 1C), (vi) resistance to pepsin (see, Example 1C), and (vii) resistance to pancreatin (see, Example 1C).

Briefly, isomaltase expression yield was determined by SDS PAGE densitometry of soluble E. coli cell lysates. Activity assays were preformed using a coupled enzyme reaction employing glucose oxidase and horseradish peroxidase in the presence of Amplex Red to detect glucose formed upon hydrolysis of isomaltose as described above. Given that the proteins were not purified at this stage, specific activities were approximated based on the amount of isomaltase protein in reactions as estimated by SDS PAGE where one unit of enzyme is assumed to consume one μmole isomaltose per min at 37° C. Stability assays were performed by pretreating variant isomaltases under activity degrading conditions and estimating fractional residual activity in the hydrolysis of p-nitrophenyl-alpha-D-glucopyranoside.

Based on this evaluation, the top distinct amino acid substitutions in L. fermentum isomaltase are set forth in TABLE 2.

TABLE 2
Top Amino Acid Substitutions

| | |
|---|---|
| R132K | A531D |
| T157A | G533E |
| E275D | E543D |
| L366M | G547S |
| E498D | |

Of the designed variants, the top three combinations of amino acid substitutions were identified, and are set forth in TABLE 3.

TABLE 3

| | Amino Acid | Amino Acid | Amino Acid |
|---|---|---|---|
| Variant No. | Change 1 | Change 2 | Change 3 |
| V131 | R132K | A531D | E498D |
| V107 | T157A | E275D | L366M |
| V177 | G533E | E543D | G547S |

An analysis of the recombinant mutant L. fermentum isomaltases revealed that variant V131, which contained three substitutions (R132K, A531D, and E498D) had the best overall positive effects across the conditions tested. Exemplary results for the top performing variants are shown in TABLE 4.

TABLE 4

| | | | | Stability | | | |
|---|---|---|---|---|---|---|---|
| | | | | pH | | Pepsin^{d} | |
| | Activityª (U/mg) | | | 4^{b} (T_{1/2}, | Tm^{c} | (T_{1/2}, | Pancreatin^{e} |
| Variant | pH 4 | pH 6 | pH 7 | min) | (° C.) | min) | (T_{1/2}, min) |
| Wild type | 58 | 77 | 70 | 99 | 51 | 6 | 35 |
| V107 | 51 | 64 | 60 | 119 | 51 | 9 | 40 |
| V131 | 56 | 81 | 73 | 94 | 50 | 8 | 70 |
| V177 | 62 | 82 | 76 | 113 | 50 | 7 | 45 |

^{a}Specific activity was estimated as μmole isomaltose consumed per minute per mg of isomaltase in reactions. Expression and reactions were performed in triplicate and averages shown. The coefficient of variation was less than 10% for all variants.
^{b}Stability at pH 4 was measured as the half-life in minutes of activity in pretreatment at 37° C. in SGF4. The coefficient of variation was less than 15% for all variants.
^{c}Tm was estimated as the temperature at which fractional residual activity was 0.5 after 30 minutes of pretreatment. The standard error was less than 0.4° C. for all variants.
^{d}Half-life in minutes of activity upon pepsin pretreatment. The isomaltase enzymes were treated at 37° C. with 20 μg/mL pepsin in SGF4, and activity was assayed at timepoints using the pNPG method. The coefficient of variation was less than 10% for all variants.
^{e}Half-life in minutes of activity upon pancreatin pretreatment. The Isomaltase enzymes were treated at 37° C. with 400 μg/mL pancreatin in SIF7 and activity was assayed at timepoints using the pNPG method. The coefficient of variation was less than 5% for all variants.

The relative impact of each substitution on each target property was estimated by modeling, and the impact of exemplary substitutions showing significant contribution to at least one of the key isomaltase properties is summarized in TABLE 5, where the substitutions listed are those that show a clear statistically significant impact (++ or +++) on at least one property or show a possible positive impact (+) on at least one property without a significant negative impact on other properties.

TABLE 5

| | Pepsin/low | | | |
|---|---|---|---|---|
| | pH | Pancreatin | | Specific |
| Substitution | Stability | Stability | Thermostability | Activity |
| A531D | ++ | +++ | −− | |
| G547S | + | +++ | − | |
| L366M | +++ | ++ | + | |
| R132K | +++ | +++ | + | |
| T157A | ++ | ++ | +++ | − |
| E275D | | + | | |
| G533E | + | | −− | |
| E543D | ++ | ++ | ++ | −− |

The substitutions in V131 (see, TABLE 2 supra) were selected for further engineering given their impact on one or more of activity at pH 4, activity at pH 7, thermostability, stability at pH 4, resistance to pepsin, and resistance to pancreatin.

### Example 1A—Isomaltase Cloning, Expression and Purification

This example describes the cloning, expression and purification of isomaltase variants.

Briefly, expression constructs encoding recombinant bacterial L. fermentum isomaltases were constructed for expression in E. coli. Expression constructs were designed with or without a C-terminal or N-terminal 6×-His affinity tag and were tested for expression and tolerance of the added His tag. The nucleic acid sequences of each expression construct were optimized for high expression in the corresponding host. The synthesized nucleic acid sequences encoding for the isomaltases were cloned into pD861-SR (a high-copy vector utilizing a strong rhamnose-inducible promoter, E. coli) or pD861-CH (for a C-terminal 6× His-tag, E. coli).

Competent E. coli BL21 cells (Agilent) were transformed with each construct using standard procedures and the resulting transformants were selected by plating on Luria-broth (LB) agar plates containing 30 μg/mL kanamycin.

Two or three colonies for each construct were isolated and each used to inoculate 500 μL of LB media with 30 μg/mL kanamycin in wells of culture well-plates. Inoculated culture well-plates were incubated at 37° C. with shaking at 1,000 rpm overnight. Then an aliquot each overnight culture was used to inoculate 500 μL of LB media with 30 μg/mL kanamycin in a deep-well microplate well. The cultures were incubated at 37° C. with shaking at 1,000 rpm. The optical density (OD) at 600 nm was monitored and cultures were induced at OD 0.8-1 with the addition of rhamnose to 4 mM. Cells were harvested by centrifugation 3 hours after induction and lysed in a total volume of 500 μL (equivalent to culture volume) using lysozyme and detergent by standard methods (Epicentre). The resulting lysates were clarified by centrifugation and stored at 4° C. before use in activity and stability assays.

His-tagged isomaltases were purified by immobilized metal affinity chromatography using standard methodology (Qiagen, Product ID: 30210). Peak elution fractions were buffer-exchanged into SIF7 buffer (123 mM NaCl, 50 mM HEPES, pH 7) with 1 mM DTT using a desalting column (Thermo Scientific, Product ID: 89882).

Expression yield was determined by densitometry of polyacrylamide gels. Samples of soluble culture lysates were subjected to polyacrylamide gel electrophoresis under denaturing, reduced conditions using standard methods. Gels were stained (SimplyBlue Safestain, ThermoFisher) and scanned. Full-length isomaltase band intensity relative to BSA standards was used to estimate protein concentration.

### Example 1B—Isomaltase Activity Assay

This example describes an isomaltase assay for determining isomaltase activity against the standard substrate isomaltose.

Reactions with isomaltase were monitored by measuring glucose formation using a coupled enzyme assay system of glucose oxidase (Gox), horseradish peroxidase (HRP) and Amplex Red (GOX/HRP method). Briefly, the GOX/HRP method detects glucose liberated by the action of hydrolase enzymes on carbohydrates, under specific pH and temperatures. Isomaltase hydrolyzes isomaltose to glucose. Upon reaction with a glucose, Gox produces H₂O₂. HRP and the H₂O₂ turn non-fluorescent Amplex red into the red fluorescent Resorufin that can be monitored.

Standard reactions for isomaltase activity screening contained 5% lysate (˜2-5 μg/mL isomaltase), 10 mM isomaltose and either SGF4 buffer (237 mM NaCl, 50 mM glycine, pH 4), SGF4.8 buffer (237 mM NaCl, 50 mM sodium acetate, pH 4.8), SIF6 buffer (123 mM NaCl, 50 mM MES, pH 6), or SIF7 buffer (123 mM NaCl, 50 mM sodium phosphate, pH 7). Reactions were incubated at 37° C. for 15 minutes and then diluted 100-fold into a glucose assay cocktail containing 400 mU/mL glucose oxidase (Sigma-Aldrich), 60 mU/mL horseradish peroxidase (ThermoFisher) and 50 μM Amplex UltraRed (ThermoFisher) at room temperature. Fluorescence increases with time was measured using a plate reader with an excitation wavelength of 530 nm and an emission wavelength of 590 nm. Glucose present was estimated based on glucose standards measured in parallel.

For certain assays of residual activity after pretreatments, hydrolysis of the substrate p-nitrophenyl-alpha-D-glucopyranoside was used (pNPG method). Reactions typically contained 0.25% isomaltase lysate or ˜0.25 μg/ml isomaltase in SIF7 buffer. In the assay pNPG was hydrolyzed to a glucopyranoside and p-nitrophenol that has a yellow color and can be monitored at 405 nm. Hydrolysis was monitored with a plate reader as an increase in absorbance at 405 nm.

### Example 1C—Isomaltase Stability Assays

This example describes an assay to determine isomaltase survivability in high temperature, low pH, and proteolytic (such as pepsin and pancreatin) conditions.

Thermostability was assayed by treatment of isomaltase samples at elevated temperature for 30 minutes prior to measuring residual activity using the pNPG method as described above. For screening, a single temperature was used for preincubation of all variants and fractional residual activity was determined. For measurements of Tm, a range of temperatures were used for pretreatment. Residual activity as a function of temperature was fit to estimate Tm as the temperature at which 50% of activity is lost.

For low pH tolerance, culture lysates or purified enzyme fractions were diluted 20-fold into SGF buffer (237 mM NaCl, 50 mM sodium acetate, pH 3.8-4) and incubated at 37° C. The treated samples were then diluted 20-fold into room temperature SIF7 buffer and residual activity was assayed using the pNPG method as described above.

Resistance to pepsin was assayed by treating isomaltase lysates with soluble pepsin from porcine gastric mucosa (Sigma) and measuring activity loss with time. Pepsin stock was prepared by dissolving pepsin powder at 3.2 mg/mL in SGF4 buffer. Culture supernatants were diluted 20-fold into SGF4 buffer containing pepsin at indicated levels (typically 10-20 μg/mL) and incubated for 2 hours at 37° C. At various timepoints the treated enzyme was then diluted 20-fold and residual activity was measured using the pNPG method as described above.

Resistance to pancreatin was assayed by treating the isomaltase variants with soluble porcine pancreatin in SIF6 buffer at 37° C. and measuring activity loss with time. Pancreatin (Sigma-Aldrich) was prepared by dissolving pancreatin powder at 10 mg/mL in SIF7 buffer. The mixture was heated to 37° C. for 10 minutes and then clarified by centrifugation at 16100×g. Soluble protein was estimated by the Bradford method (Coomassie Plus, ThermoFisher). The reported pancreatin concentrations in treatments are based on measured soluble protein. Some variation was seen in solubilized protease activity between lots of purchased pancreatin and individual preparations. For stability studies, culture supernatants were diluted 20-fold into SIF7 buffer containing pancreatin and incubated for up to 3 hours at 37° C. At various timepoints the treated enzyme was then diluted 20-fold and residual activity was measured using the pNPG method as described above.

From the standpoint of survivability, it was desirable that improvements to pH survivability not adversely affect performance against protease and vice versa.

### Example 2—Isomaltase Engineering

This example illustrates L. fermentum isomaltase engineering using V131 (from Example 1) as a starting point because this variant showed a promising combination of properties (see, TABLE 4, above). In this phase of engineering, variants of V131 were engineered with multiple new amino acid substitutions so that amino acid substitutions were retained, and new substitutions were sampled in additional variants.

The top selected distinct amino acid substitutions in L. fermentum isomaltase resulting from this design are set forth in TABLE 6.

TABLE 6
Top Amino Acid Substitutions

| | | |
|---|---|---|
| T89A | K269R | A531D |
| K115I | A444G | G547S |
| R132K | S455A | F560L |
| E178P | E498D | |
| D186E | E501D | |

Of the variants tested, the top four performers (each comprising 4-6 substitutions relative to wild type) are set forth in TABLE 7.

TABLE 7

| Variant | Amino Acid | Amino Acid | Amino Acid | Amino Acid | Amino Acid | Amino Acid |
|---|---|---|---|---|---|---|
| No. | 1 Substn | 2 Substn | 3 Substn | 4 Substn | 5 Substn | 6 Substn |
| V202 | R132K | E498D | A531D | T89A | E178P | F560L |
| V203 | K115I | R132K, | A444G, | A531D | | |
| V210 | R132K | S455A | E498D | A531D | K115I | D186E |
| V237 | R132K | E498D | A531D | T82A | E178P | E501D |

The engineered variants were expressed and tested essentially as described in Example 1. In this example, the low pH stability treatment was lowered to pH 3.9 and the pancreatin treatment level was increased to 800 μg/mL. The impact of different substitutions on the key properties were determined from data models. An analysis of the recombinant mutant L. fermentum isomaltases revealed that variant V203 had the best overall positive effects across the conditions tested. V203 contained four substitutions (K115I, R132K, A444G, and A531D), was used as the starting point for a further phase of enzyme engineering.

Exemplary results for four of the variants from this phase relative to the parent (V131) are shown in TABLE 8, where improvements in pancreatin stability and low pH stability was observed.

TABLE 8

| | | | | Stability | | | |
|---|---|---|---|---|---|---|---|
| | | | | pH 3.9^{b} | | Pepsin^{d} | Pancreatin^{e} |
| | Activityª (U/mg) | | | (T_{1/2}, | Tm^{c} | (T_{1/2}, | (T_{1/2}, |
| Variant | pH 4 | pH 6 | pH 7 | min) | (° C.) | min) | min) |
| V131 | 39 | 48 | 44 | 39 | 50 | 16 | 54 |
| V202 | 39 | 51 | 47 | 58 | 50 | 14 | 55 |
| V203 | 34 | 50 | 45 | 49 | 52 | 13 | 94 |
| V210 | 33 | 41 | 38 | 51 | 52 | 15 | 76 |
| V237 | 34 | 41 | 37 | 65 | 50 | 15 | 50 |

^{a}Specific activity was estimated as μmole isomaltose consumed per minute per mg of isomaltase in reactions. Expression and reactions were performed in duplicate and averages shown. The coefficient of variation was less than 15% for all variants.
^{b}Stability at pH 3.9 was measured as the half-life in minutes of activity in pretreatment at 37° C. in SGF, pH 3.9. The coefficient of variation was less than 6% for all variants.
^{c}Tm was estimated as the temperature at which fractional residual activity was 0.5 after 30 minutes of pretreatment. The standard error was less than 0.4° C. for all variants.
^{d}Half-life in minutes of activity upon pepsin pretreatment. Isomaltases were treated at 37° C. with 10 μg/mL pepsin in SGF4 and activity was assayed at timepoints using the pNPG method. The coefficient of variation was less than 10% for all variants.
^{e}Half-life in minutes of activity upon pancreatin pretreatment. Isomaltases were treated at 37° C. with 800 μg/mL pancreatin in SIF7 and activity was assayed at timepoints using the pNPG method. The coefficient of variation was less than 10% for all variants.
^{f}Soluble expression was measured as μg isomaltase in the soluble fraction of culture lysates per mL of culture as estimated by PAGE densitometry. Coefficient of variation was less than 20% for all variants.

The relative impact of each substitution on each target property was estimated by modeling and the impact of exemplary substitutions showing significant contribution to at least one of the key isomaltase properties is summarized in TABLE 9, where the substitutions listed are those that show a clear statistically significant impact (++ or +++) on at least one property or show a possible positive impact (+) on at least one property without a significant negative impact on other properties.

TABLE 9

| | Pepsin | Low pH | Pancreatin | | Specific |
|---|---|---|---|---|---|
| Substitution | Stability | Stability | Stability | Thermostability | Activity |
| T89A | + | | | + | |
| K115I | | ++ | +++ | +++ | ++ |
| E178P | ++ | +++ | − | | + |
| D186E | | | + | + | |
| K269R | | | + | | +++ |
| S455A | + | + | + | + | |
| E501D | | | | + | + |
| A531D | | | ++ | −− | |
| A444G | − | + | +++ | ++ | |
| F560L | ++ | + | + | + | |

### Example 3—Isomaltase Engineering—Phase 3

This example describes further L. fermentum isomaltase engineering using V203 from Example 2 as a starting point for further engineering because this variant showed a promising combination of properties, including pancreatin stability and low pH stability (see TABLE 8, above).

In this example, new variants were designed with the goals of (i) improving low pH stability and (ii) pepsin resistance. With regard to improving low pH stability, substitutions predicted to reduce the number of pepsin sensitive sites based on known pepsin specificity were selected. With regard to improving pepsin resistance, a bias toward the modification of residues with possible pKa in the 3-5 range was used given that protonation of these amino acids could potentially result in instability at a pH of 5 or below. In this example, 95 variants were designed wherein 18 substitutions from phase 2 were retained and 30 new substitutions were sampled in different combinations.

The top amino acid substitutions identified are summarized in TABLE 10.

TABLE 10
Amino Acid Substitutions

| | | | |
|---|---|---|---|
| S12A | V168K | L366M | A531D |
| T82A | E178D | L394I | G535S |
| E86Q | H182R | I421A | E543N |
| T89A | A211E | A444G | R551N |
| E93K | D226S | S455A | E554Q |
| K115I | Q246R | M486I | K556I |
| E122D | K269R | E498N | F560L |
| R132K | E275D | Q502E | F560I |
| D136N | P309R | T514K | F560V |
| T157A | E310A | E524Q | |

A number of variants having beneficial properties based on different combinations of amino acid substitutions (“AA Sub”) are summarized in TABLE 11.

TABLE 11

| Variant | AA | AA | AA | AA | AA | AA | AA |
|---|---|---|---|---|---|---|---|
| No. | Sub 1 | Sub 2 | Sub 3 | Sub 4 | Sub 5 | Sub 6 | Sub 7 |
| V301 | S12A | T89A | K115I | R132K | E275D | A444G | A531D |
| V302 | T89A | K115I | R132K | A444G | S455A | A531D | F560I |
| V303 | T89A | K115I | R132K | D226S | P309R | A444G | A531D |
| V305 | T82A | K115I | R132K | E275D | A444G | S455A | A531D |
| V306 | K115I | R132K | H182R | A444G | T514K | A531D | E543N |
| V308 | E93K | K115I | T157A | A444G | A531D | | |
| V311 | K115I | R132K | A211E | K269R | E275D | A444G | A531D |
| V312 | E86Q | K115I | R132K | A444G | M486I | A531D | F560V |
| V313 | K115I | R132K | T157A | D226S | A444G | E524Q | A531D |
| V315 | E93K | K115I | R132K | D136N | A444G | A531D | K556I |
| V317 | K115I | R132K | T157A | H182R | A444G | S455A | A531D |
| V318 | K115I | R132K | E275D | A444G | A531D | R551N | F560V |
| V320 | K115I | E275D | P309R | A444G | A531D | | |
| V321 | K115I | R132K | E178D | L366M | A444G | A531D | F560I |
| V322 | S12A | K115I | R132K | H182R | K269R | A444G | A531D |
| V327 | T89A | E93K, | K115I | R132K | H182R | A444G | A531D |
| V329 | K115I | A211E | L366M | A444G | A531D | | |
| V335 | T82A | K115I | R132K | A444G | Q502E | A531D | F560L |
| V336 | S12A | K115I | R132K | E178D | E310A | A444G | A531D |
| V337 | K115I | R132K | Q246R | A444G | A531D | G535S | K556I |
| V344 | E93K | K115I | R132K | E275D | A444G | A531D | F560I |
| V345 | T82A | E93K | K115I | R132K | L366M | A444G | A531D |
| V346 | E93K | K115I | R132K | V168K | A444G | M486I | A531D |
| V349 | S12A | T82A | K115I | R132K | A531D | | |
| V351 | E86Q | K115I | R132K | E275D | L366M | A444G | A531D |
| V352 | E93K | K115I | R132K | A444G | G535S | | |
| V353 | K115I | R132K | D226S | I421A | A444G | A531D | F560L |
| V354 | S12A | E93K | K115I | R132K | A444G | S455A | A531D |
| V359 | K115I | R132K | K269R | A444G | A531D | E554Q | K556I |
| V361 | K115I | E122D | R132K | E310A | A444G | A531D | E543N |
| V366 | S12A | K115I | R132K | D136N | A444G | A531D | G535S |
| V370 | K115I | R132K | E178D | E498N | A531D | | |
| V371 | K115I | R132K | K269R | A444G | S455A | | |
| V372 | T82A | K115I | R132K | D226S | L394I | A444G | A531D |
| V374 | K115I | R132K | S455A | A531D | F560L | | |
| V376 | K115I | R132K | E310A | A444G | Q502E | T514K | A531D |
| V378 | S12A | K115I | R132K | L366M | A444G | | |
| V385 | T82A | T89A | K115I | R132K | A444G | | |
| V391 | K115I | R132K | T157A | A444G | A531D | G535S | F560L |
| V392 | T82A | K115I | R132K | Q246R | A444G | A531D | E543N |
| V393 | E93K | K115I | R132K | A211E | A444G | T514K | A531D |
| V395 | K115I | R132K | E178D | A211E | A444G | A531D | F560L |

The variants were expressed and tested essentially as described in Example 2. The impact of substitutions on the key properties were determined from data modeling. Promising variants were retested to confirm relative improvements observed in the screen data. To determine the impact of lysate components on stability and activity measurements, the top variants were also made with C-terminal His-tags and purified by IMAC. The resulting lysates of the tagged and untagged variants showed similar activities and stabilities. However, the purified enzymes showed a drop in measures of performance, Results for exemplary variants are shown in TABLE 12.

TABLE 12

| | | | | Stability | | | |
|---|---|---|---|---|---|---|---|
| | Activityª (U/mg) | | | pH 3.9^{b} | Tm^{c} | Pepsin^{d} | Pancreatin^{e} |
| Variant | pH 4 | pH 6 | pH 7 | (% Res) | (° C.) | (% Res) | (T_{1/2}, min) |
| V203 | 65 | 84 | 82 | 59 | 53 | 16 | 101 |
| V303 | 61 | 73 | 69 | 39 | 53 | 23 | 151 |
| V313 | 60 | 69 | 67 | 35 | 53 | 25 | 195 |
| V320 | 75 | 95 | 90 | 58 | 52 | 13 | 66 |
| V322 | 70 | 87 | 85 | 55 | 52 | 18 | 87 |
| V353 | 69 | 78 | 74 | 38 | 54 | 33 | 193 |
| V372 | 41 | 44 | 40 | 52 | 55 | 40 | 582 |

^{a}Specific activity was estimated as μmole isomaltose consumed per minute per mg of isomaltase in reactions. Expression and reactions were performed in duplicate and averages shown. The coefficient of variation was less than 20% for all variants.
^{b}Stability at pH 3.9 was measured as the percent residual activity after pretreatment for 10 minutes at 37° C. in SGF, pH 3.9.
^{c}Tm was estimated as the temperature at which fractional residual activity is 0.5 after 30 minutes of pretreatment. The standard error was less than 0.4° C. for all variants.
^{d}Percent residual activity after pretreatment for 10 minutes at 37° C. with 20 μg/mL pepsin in SGF4.
^{e}Half-life in minutes of activity upon pancreatin pretreatment. Isomaltases were treated at 37° C. with 800 μg/mL pancreatin in SIF7 and activity was assayed at timepoints using the pNPG method.
^{f}Soluble expression was measured as μg isomaltase in the soluble fraction of culture lysates per mL of culture as estimated by PAGE densitometry. The coefficient of variation was less than 25% for all variants.

Results of key isomaltase properties for the most promising select variants are summarized in TABLE 13, where the variants listed show a clear statistically significant impact (++ or +++) on at least one property or show a possible positive impact (+) on at least one property without a significant negative impact on other properties.

TABLE 13

| | Specific | | Pancreatin | Low pH | Pepsin |
|---|---|---|---|---|---|
| Variant | Activity | Thermostability | Resistance | Stability | Resistance |
| V301 | + | | ++ | | + |
| V302 | + | | ++ | + | + |
| V303 | + | | ++ | | + |
| V305 | + | | ++ | | + |
| V306 | | − | ++ | + | + |
| V308 | + | | ++ | | + |
| V311 | + | | ++ | | + |
| V312 | + | | ++ | + | + |
| V313 | + | | ++ | | + |
| V315 | | − | ++ | + | + |
| V317 | + | − | ++ | + | + |
| V318 | | | ++ | | + |
| V320 | + | | + | | |
| V321 | | | ++ | + | + |
| V322 | + | − | ++ | | + |
| V327 | + | − | ++ | + | + |
| V329 | + | − | ++ | | + |
| V335 | + | | ++ | + | + |
| V336 | | − | ++ | + | + |
| V337 | − | − | ++ | + | + |
| V344 | | | ++ | + | + |
| V345 | + | − | ++ | + | + |
| V346 | + | − | ++ | − | |
| V349 | | − | ++ | + | + |
| V351 | + | − | ++ | | + |
| V352 | + | | ++ | + | + |
| V353 | + | | ++ | | + |
| V354 | + | − | ++ | | |
| V359 | − | − | + | | ++ |
| V361 | + | | ++ | + | + |
| V366 | + | − | ++ | + | + |
| V370 | | − | + | + | ++ |
| V371 | + | | + | | |
| V372 | − | | ++ | + | ++ |
| V374 | + | | ++ | + | + |
| V376 | + | | ++ | + | + |
| V378 | + | | + | + | + |
| V385 | | | + | + | + |
| V391 | + | | ++ | + | + |
| V392 | + | − | ++ | + | + |
| V393 | | | ++ | | + |
| V395 | | | ++ | | + |

A significant improvement was observed in pancreatin stability and specific activity among the retested variants although the top stable variant (V372) still displayed relatively poor specific activity, whereas the top active variants showed poor stability. Beneficial substitutions including K115I, R132K, A444G, and A531ID were present in a majority of the variants. Variant V353 had good overall properties and was used as a starting point for another phase of engineering.

### Example 4—Isomaltase Engineering—Phase 4

In this phase, V353 from Example 3 was chosen as a starting point for further engineering as it showed a promising combination of stability and activity.

In this Example, 32 variants were designed based on V353 in which substitutions with a positive impact based on the studies set forth in Example 3 studies were recombined in different combinations, without the introduction of any new amino acid substitutions. Briefly, 32 combinations (variants) of amino acids substitutions were designed to promote the simultaneous improvement of specific activity and pancreatin stability. Eleven total amino acid substitutions were sampled including a reversion of one substitution in V353 (namely, R132K) which had shown a positive impact on stability but a negative impact on activity

The top selected distinct amino acid substitutions in L. fermentum isomaltase used in this phase are listed in TABLE 14.

TABLE 14
Amino Acid Substitutions

| | |
|---|---|
| T89A | E310A |
| E93K | L366M |
| K115I | I421A |
| E122D | A444G |
| R132K | E524Q |
| H182R | A531D |
| A211E | F560L |
| D226S | F560V |
| E275D | |

A listing of the combinations of the amino acid substitutions (AA Sub) evaluated in this phase is set forth in TABLE 15.

TABLE 15

| Variant | AA Sub | AA Sub | AA Sub | AA Sub | AA Sub | AA Sub | AA Sub | AA Sub | AA Sub | AA Sub |
|---|---|---|---|---|---|---|---|---|---|---|
| No. | 1 | 2 | 3 | 4 | 5 | 6 | 7 | 8 | 9 | 10 |
| V401 | E93K | K115I | R132K | D226S | E310A | L366M | I421A | A444G | A531D | F560L |
| V402 | E93K | K115I | R132K | D226S | E310A | I421A | A444G | E524Q | A531D | F560L |
| V403 | E93K | K115I | R132K | D226S | E310A | I421A | A444G | A531D | F560V | |
| V404 | E93K | K115I | R132K | D226S | L366M | I421A | A444G | E524Q | A531D | F560L |
| V405 | E93K | K115I | R132K | D226S | L366M | I421A | A444G | A531D | F560V | |
| V406 | E93K | K115I | R132K | D226S | I421A | A444G | E524Q | A531D | F560V | |
| V407 | K115I | R132K | D226S | E310A | L366M | I421A | A444G | E524Q | A531D | F560L |
| V408 | K115I | R132K | D226S | E310A | L366M | I421A | A444G | A531D | F560V | |
| V409 | K115I | R132K | D226S | E310A | I421A | A444G | E524Q | A531D | F560V | |
| V410 | K115I | R132K | D226S | L366M | I421A | A444G | E524Q | A531D | F560V | |
| V411 | E93K | K115I | R132K | A211E | D226S | I421A | A444G | E524Q | A531D | F560V |
| V412 | E93K | K115I | R132K | D226S | L366M | I421A | A444G | E524Q | A531D | F560V |
| V413 | E93K | K115I | R132K | D226S | E275D | I421A | A444G | E524Q | A531D | F560V |
| V414 | T89A | E93K | K115I | R132K | D226S | I421A | A444G | E524Q | A531D | F560V |
| V415 | E93K | K115I | R132K | H182R | D226S | I421A | A444G | E524Q | A531D | F560V |
| V416 | E93K | K115I | E122D | R132K | D226S | I421A | A444G | E524Q | A531D | F560V |
| V417 | E93K | K115I | D226S | I421A | A444G | E524Q | A531D | F560V | | |
| V418 | E93K | K115I | D226S | I421A | A444G | E524Q | A531D | F560L | | |
| V419 | K115I | A211E | D226S | I421A | A444G | A531D | F560V | | | |
| V420 | K115I | D226S | L366M | I421A | A444G | E524Q | A531D | F560L | | |
| V421 | K115I | A211E | D226S | I421A | A444G | E524Q | A531D | F560L | | |
| V422 | K115I | R132K | D226S | E310A | L366M | I421A | A444G | A531D | F560L | |
| V423 | E93K | K115I | D226S | E310A | I421A | A444G | A531D | F560L | | |
| V424 | K115I | D226S | E310A | I421A | A444G | A531D | F560V | | | |
| V425 | E93K | K115I | D226S | L366M | I421A | A444G | A531D | F560L | | |
| V426 | K115I | D226S | I421A | A444G | E524Q | A531D | F560V | | | |
| V427 | K115I | D226S | E310A | I421A | A444G | E524Q | A531D | F560L | | |
| V428 | K115I | A211E | D226S | L366M | I421A | A444G | A531D | F560L | | |
| V429 | K115I | A211E | D226S | E310A | I421A | A444G | A531D | F560L | | |
| V430 | E93K | K115I | D226S | I421A | A444G | A531D | F560V | | | |
| V431 | E93K | K115I | A211E | D226S | I421A | A444G | A531D | F560L | | |
| V432 | K115I | D226S | L366M | I421A | A444G | A531D | F560V | | | |

Among the 32 variants produced, moderate improvements were observed in activity and stability. Exemplary results for a select number of variants relative to V322, V353, and V372 are shown in TABLE 16.

TABLE 16

| | | | | Stability | | | |
|---|---|---|---|---|---|---|---|
| | Activityª (U/mg) | | | pH 3.8^{b} | Tm^{c} | Pepsin^{d} | Pancreatin^{e} |
| Variant | pH 4 | pH 6 | PH 7 | (T_{1/2}, min) | (° C.) | (T_{1/2}, min) | (T_{1/2}, min) |
| V322 | 61 | 84 | 78 | 9.5 | 51 | 3.5 | 76 |
| V353 | 58 | 80 | 71 | 5.5 | 53 | 5.0 | 138 |
| V372 | 33 | 40 | 33 | 5.5 | 55 | 5.5 | 311 |
| V408 | 76 | 111 | 95 | 9 | 53 | 11.5 | 134 |
| V413 | 62 | 87 | 73 | 4.5 | 53 | 5.5 | 139 |
| V414 | 60 | 90 | 76 | 6.0 | 53 | 5.0 | 129 |
| V425 | 77 | 103 | 93 | 5.5 | 52 | 8.0 | 142 |
| V432 | 65 | 96 | 85 | 6 | 53 | 8.5 | 130 |

^{a}Specific activity was estimated as μmole isomaltose consumed per minute per mg of isomaltase in reactions. Expression and reactions were performed in duplicate and averages shown. The coefficient of variation was less than 20% for all variants.
^{b}Stability at pH 3.8 was measured as the half-life in minutes of activity in pretreatment at 37° C. in SGF, pH 3.8.
^{c}Tm was estimated as the temperature at which fractional residual activity is 0.5 after 30 minutes of pretreatment. The standard error was less than 0.5° C. for all variants.
^{d}Half-life in minutes of activity upon pepsin pretreatment. Isomaltases were treated at 37° C. with 20 μg/ml pepsin in SGF4 and activity was assayed at timepoints using the pNPG method. The coefficient of variation was less than 21% for all variants.
^{e}Half-life in minutes of activity upon pancreatin pretreatment. Isomaltases were treated at 37° C. with 800 μg/mL pancreatin in SIF7 and activity was assayed at timepoints using the pNPG method. The coefficient of variation was less than 15% for all variants.

Results of key isomaltase properties for select variants are summarized in TABLE 17, wherein the isomaltase variants listed show a statistically significant impact (++ or on at least one property or show a possible positive impact (+) on at least one property without a significant negative impact on other properties.

TABLE 17

| Variant | Specific | | Pancreatin | Low pH | Pepsin |
|---|---|---|---|---|---|
| Name | Activity | Thermostability | Resistance | Stability | Resistance |
| V401 | + | | ++ | + | ++ |
| V402 | | | ++ | | ++ |
| V403 | | | ++ | + | ++ |
| V404 | + | | ++ | | ++ |
| V405 | + | − | ++ | + | ++ |
| V406 | | | ++ | | ++ |
| V407 | + | − | ++ | + | ++ |
| V408 | + | | ++ | + | ++ |
| V409 | + | | ++ | + | ++ |
| V410 | + | | ++ | | ++ |
| V411 | | | ++ | | ++ |
| V412 | + | | ++ | | ++ |
| V413 | | | ++ | | ++ |
| V414 | + | | ++ | | ++ |
| V415 | + | | ++ | | ++ |
| V416 | | | ++ | | ++ |
| V417 | + | | ++ | | ++ |
| V418 | + | | ++ | | ++ |
| V419 | | | ++ | − | ++ |
| V420 | + | − | ++ | | ++ |
| V421 | | | ++ | − | ++ |
| V422 | + | | ++ | + | ++ |
| V423 | + | | ++ | | ++ |
| V424 | | | ++ | | ++ |
| V425 | + | | ++ | | ++ |
| V426 | | | ++ | | ++ |
| V427 | | | ++ | | ++ |
| V428 | + | | ++ | | ++ |
| V429 | | | ++ | | ++ |
| V430 | + | | ++ | | ++ |
| V431 | | | ++ | − | ++ |
| V432 | + | − | ++ | + | ++ |

Together, these studies identified several substitutions that can be used alone or in combination to positively impact activity, low pH stability, proteolytic stability, and thermostability of L. fermentum isomaltase. All the variants designed in this phase showed improvement relative to wild type. Variant V408 showed all-around significant improvement relative to wild type isomaltase.

Beneficial substitutions including K115I, D226S, I421A, A444G, A531D, and either F560V or F560L were present in all 32 variants.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

SEQUENCE LISTING

SEQ ID NO: 1 (L. fermentum isomaltase)
MIYTPKWWWQNSVVYQVYPRSFQDSNHDGIGDLKGIISRLDYIKKLGADIIWLNPIYRSPNV
DNGYDISDYRAIDPTFGSLTDFKELLTKAHELGLKIMMDLVVNHSSDENEWFKQSRQGKENP
YRDYYIWRDPVDGHEPTNWGSYFSGSAWQYDETSGQYYLHLFAVKQPDLNWENEAVRHSVYD
IMNWWADLGVDGFRMDVINLISKPAVYKDVPTAPGMQYGDVEPVVANGHRMHEFLQEMHQAV
MAKHDLVTVGETPGATTDDAKKYANLEQTELNMVFEFEHVGLDGNDNPALGKWSDKKVSLPE
LRDNLVKWQTQLNGKAWNSLYWNNHDQPRVVSRFGNDDPKYRVVSAKMLATMLHCLQGTPYI
YAGEELGMTNTTFNSLSDYRDLESINAYHQLVDEEHLVDGKTMSRYLAIHSRDNARTPMQWD
DSKNAGFSDAEPWIAVNPNYSEINAKAALADPSSVFYHYQKLIQMRHDLPVMTEGKFALVNG
NELDEQVFAYTRDDGETTLLVVANFTKETIKREYAAGQGKLLLSNYEDDMGETLRPYEAKVY
EFSSKR
SEQ ID NO: 2 (L. fermentum isomaltase DNA, AVAB01000001.1:867450-
869144)
ATGATATATACACCAAAATGGTGGTGGCAAAATTCAGTTGTCTACCAAGTCTATCCACGGAG
TTTTCAAGACAGCAATCATGATGGCATTGGTGATTTAAAAGGAATCATCAGTCGGCTTGACT
ATATTAAAAAACTAGGTGCTGATATTATCTGGCTCAATCCAATCTACCGTTCGCCAAACGTT
GATAATGGGTATGACATCAGTGATTATCGGGCAATTGATCCAACTTTTGGCTCATTGACCGA
CTTTAAGGAGCTGCTGACAAAGGCTCATGAACTTGGATTAAAAATAATGATGGATCTGGTGG
TCAATCACTCATCAGATGAAAATGAATGGTTTAAGCAAAGCCGCCAAGGAAAAGAAAATCCA
TACCGTGACTATTATATCTGGCGTGATCCAGTCGATGGCCATGAACCAACTAATTGGGGATC
ATACTTCTCTGGTTCAGCTTGGCAGTACGATGAGACAAGCGGTCAATACTACCTTCACCTGT
TCGCTGTAAAGCAGCCGGATCTAAACTGGGAAAATGAAGCCGTTCGTCATTCGGTATACGAC
ATCATGAATTGGTGGGCAGATCTCGGTGTCGATGGATTTCGAATGGATGTCATCAACTTAAT
TTCAAAACCGGCTGTCTACAAAGACGTTCCAACCGCGCCCGGAATGCAGTATGGCGATGTCG
AGCCGGTTGTTGCCAATGGTCATCGGATGCACGAATTCCTTCAAGAAATGCATCAAGCTGTA
ATGGCCAAGCATGATTTGGTAACAGTTGGCGAAACACCTGGTGCTACAACAGATGATGCCAA
GAAGTACGCCAACCTTGAACAGACTGAGCTGAACATGGTTTTTGAGTTTGAGCATGTCGGCT
TAGACGGTAACGACAACCCAGCTTTAGGAAAATGGAGCGACAAAAAAGTCAGTCTGCCAGAA
TTGCGAGATAACCTGGTGAAATGGCAGACTCAGCTGAATGGTAAGGCTTGGAACTCGCTTTA
CTGGAACAACCATGATCAGCCACGCGTCGTATCCCGTTTTGGCAATGATGATCCAAAATATC
GTGTCGTTTCGGCTAAAATGTTGGCAACAATGCTGCACTGTCTGCAAGGCACGCCTTATATC
TACGCAGGTGAGGAACTGGGAATGACCAACACGACCTTTAACTCTTTGTCTGACTATCGTGA
TTTGGAGAGCATTAATGCCTACCATCAGCTGGTTGACGAAGAGCATCTAGTTGATGGAAAGA
CAATGAGCAGATATCTGGCAATTCATTCTCGGGACAATGCCAGAACGCCAATGCAATGGGAT
GACAGCAAGAATGCCGGATTTTCGGATGCTGAACCTTGGATCGCGGTCAATCCCAACTATTC
AGAGATCAACGCAAAGGCGGCACTGGCGGATCCATCATCCGTCTTCTATCACTACCAGAAAC
TCATTCAAATGCGCCATGACTTGCCAGTAATGACAGAAGGAAAGTTTGCGTTGGTCAATGGC
AACGAATTGGATGAGCAGGTCTTTGCTTACACGCGTGATGATGGAGAAACAACGCTGCTTGT
AGTAGCCAACTTTACTAAGGAAACAATCAAGCGAGAATACGCGGCTGGTCAAGGCAAGCTCT
TATTAAGCAACTACGAGGATGACATGGGAGAAACTCTGCGTCCATATGAAGCTAAAGTATAT
GAATTCAGTTCAAAGAGGTAA
SEQ ID NO: 3 (L. fermentum isomaltase) V131
MIYTPKWWWQNSVVYQVYPRSFQDSNHDGIGDLKGIISRLDYIKKLGADIIWLNPIYRSPNV
DNGYDISDYRAIDPTFGSLTDFKELLTKAHELGLKIMMDLVVNHSSDENEWFKQSRQGKENP
YRDYYIWKDPVDGHEPTNWGSYFSGSAWQYDETSGQYYLHLFAVKQPDLNWENEAVRHSVYD
IMNWWADLGVDGFRMDVINLISKPAVYKDVPTAPGMQYGDVEPVVANGHRMHEFLQEMHQAV
MAKHDLVTVGETPGATTDDAKKYANLEQTELNMVFEFEHVGLDGNDNPALGKWSDKKVSLPE
LRDNLVKWQTQLNGKAWNSLYWNNHDQPRVVSRFGNDDPKYRVVSAKMLATMLHCLQGTPYI
YAGEELGMTNTTFNSLSDYRDLESINAYHQLVDEEHLVDGKTMSRYLAIHSRDNARTPMQWD
DSKNAGFSDAEPWIAVNPNYSEINAKAALADPSSVFYHYQKLIQMRHDLPVMTEGKFALVNG
NDLDEQVFAYTRDDGETTLLVVANFTKETIKREYDAGQGKLLLSNYEDDMGETLRPYEAKVY
EFSSKR.
SEQ ID NO: 4 (L. fermentum isomaltase) V203
MIYTPKWWWQNSVVYQVYPRSFQDSNHDGIGDLKGIISRLDYIKKLGADIIWLNPIYRSPNV
DNGYDISDYRAIDPTFGSLTDFKELLTKAHELGLKIMMDLVVNHSSDENEWFIQSRQGKENP
YRDYYIWKDPVDGHEPTNWGSYFSGSAWQYDETSGQYYLHLFAVKQPDLNWENEAVRHSVYD
IMNWWADLGVDGFRMDVINLISKPAVYKDVPTAPGMQYGDVEPVVANGHRMHEFLQEMHQAV
MAKHDLVTVGETPGATTDDAKKYANLEQTELNMVFEFEHVGLDGNDNPALGKWSDKKVSLPE
LRDNLVKWQTQLNGKAWNSLYWNNHDQPRVVSRFGNDDPKYRVVSAKMLATMLHCLQGTPYI
YAGEELGMTNTTFNSLSDYRDLESINAYHQLVDEEHLVDGKTMSRYLAIHSRDNARTPMQWD
DSKNAGFSDGEPWIAVNPNYSEINAKAALADPSSVFYHYQKLIQMRHDLPVMTEGKFALVNG
NELDEQVFAYTRDDGETTLLVVANFTKETIKREYDAGQGKLLLSNYEDDMGETLRPYEAKVY
EFSSKR.
SEQ ID NO: 5 (L. fermentum isomaltase) V353
MIYTPKWWWQNSVVYQVYPRSFQDSNHDGIGDLKGIISRLDYIKKLGADIIWLNPIYRSPNV
DNGYDISDYRAIDPTFGSLTDFKELLTKAHELGLKIMMDLVVNHSSDENEWFIQSRQGKENP
YRDYYIWKDPVDGHEPTNWGSYFSGSAWQYDETSGQYYLHLFAVKQPDLNWENEAVRHSVYD
IMNWWADLGVDGFRMDVINLISKPAVYKDVPTAPGMQYGSVEPVVANGHRMHEFLQEMHQAV
MAKHDLVTVGETPGATTDDAKKYANLEQTELNMVFEFEHVGLDGNDNPALGKWSDKKVSLPE
LRDNLVKWQTQLNGKAWNSLYWNNHDQPRVVSRFGNDDPKYRVVSAKMLATMLHCLQGTPYI
YAGEELGMTNTTFNSLSDYRDLESINAYHQLVDEEHLVDGKTMSRYLAAHSRDNARTPMQWD
DSKNAGFSDGEPWIAVNPNYSEINAKAALADPSSVFYHYQKLIQMRHDLPVMTEGKFALVNG
NELDEQVFAYTRDDGETTLLVVANFTKETIKREYDAGQGKLLLSNYEDDMGETLRPYEAKVY
ELSSKR.
SEQ ID NO: 6 (L. fermentum isomaltase) V372
MIYTPKWWWQNSVVYQVYPRSFQDSNHDGIGDLKGIISRLDYIKKLGADIIWLNPIYRSPNV
DNGYDISDYRAIDPTFGSLADFKELLTKAHELGLKIMMDLVVNHSSDENEWFIQSRQGKENP
YRDYYIWKDPVDGHEPTNWGSYFSGSAWQYDETSGQYYLHLFAVKQPDLNWENEAVRHSVYD
IMNWWADLGVDGFRMDVINLISKPAVYKDVPTAPGMQYGSVEPVVANGHRMHEFLQEMHQAV
MAKHDLVTVGETPGATTDDAKKYANLEQTELNMVFEFEHVGLDGNDNPALGKWSDKKVSLPE
LRDNLVKWQTQLNGKAWNSLYWNNHDQPRVVSRFGNDDPKYRVVSAKMLATMLHCLQGTPYI
YAGEELGMTNTTFNSLSDYRDIESINAYHQLVDEEHLVDGKTMSRYLAIHSRDNARTPMQWD
DSKNAGFSDGEPWIAVNPNYSEINAKAALADPSSVFYHYQKLIQMRHDLPVMTEGKFALVNG
NELDEQVFAYTRDDGETTLLVVANFTKETIKREYDAGQGKLLLSNYEDDMGETLRPYEAKVY
EFSSKR.
SEQ ID NO: 7 (L. fermentum isomaltase) V408
MIYTPKWWWQNSVVYQVYPRSFQDSNHDGIGDLKGIISRLDYIKKLGADIIWLNPIYRSPNV
DNGYDISDYRAIDPTFGSLTDFKELLTKAHELGLKIMMDLVVNHSSDENEWFIQSRQGKENP
YRDYYIWKDPVDGHEPTNWGSYFSGSAWQYDETSGQYYLHLFAVKQPDLNWENEAVRHSVYD
IMNWWADLGVDGFRMDVINLISKPAVYKDVPTAPGMQYGSVEPVVANGHRMHEFLQEMHQAV
MAKHDLVTVGETPGATTDDAKKYANLEQTELNMVFEFEHVGLDGNDNPALGKWSDKKVSLPA
LRDNLVKWQTQLNGKAWNSLYWNNHDQPRVVSRFGNDDPKYRVVSAKMLATMLHCMQGTPYI
YAGEELGMTNTTFNSLSDYRDLESINAYHQLVDEEHLVDGKTMSRYLAAHSRDNARTPMQWD
DSKNAGESDGEPWIAVNPNYSEINAKAALADPSSVFYHYQKLIQMRHDLPVMTEGKFALVNG
NELDEQVFAYTRDDGETTLLVVANFTKETIKREYDAGQGKLLLSNYEDDMGETLRPYEAKVY
EVSSKR.
SEQ ID NO: 8 (L. fermentum isomaltase) V107
MIYTPKWWWQNSVVYQVYPRSFQDSNHDGIGDLKGIISRLDYIKKLGADIIWLNPIYRSPNV
DNGYDISDYRAIDPTFGSLTDFKELLTKAHELGLKIMMDLVVNHSSDENEWFKQSRQGKENP
YRDYYIWRDPVDGHEPTNWGSYFSGSAWQYDEASGQYYLHLFAVKQPDLNWENEAVRHSVYD
IMNWWADLGVDGFRMDVINLISKPAVYKDVPTAPGMQYGDVEPVVANGHRMHEFLQEMHQAV
MAKHDLVTVGETPGATTDDAKKYANLDQTELNMVFEFEHVGLDGNDNPALGKWSDKKVSLPE
LRDNLVKWQTQLNGKAWNSLYWNNHDQPRVVSRFGNDDPKYRVVSAKMLATMLHCMQGTPYI
YAGEELGMTNTTFNSLSDYRDLESINAYHQLVDEEHLVDGKTMSRYLAIHSRDNARTPMQWD
DSKNAGFSDAEPWIAVNPNYSEINAKAALADPSSVFYHYQKLIQMRHDLPVMTEGKFALVNG
NELDEQVFAYTRDDGETTLLVVANFTKETIKREYAAGQGKLLLSNYEDDMGETLRPYEAKVY
EFSSKR.
SEQ ID NO: 9 (L. fermentum isomaltase) V177
MIYTPKWWWQNSVVYQVYPRSFQDSNHDGIGDLKGIISRLDYIKKLGADIIWLNPIYRSPNV
DNGYDISDYRAIDPTFGSLTDFKELLTKAHELGLKIMMDLVVNHSSDENEWFKQSRQGKENP
YRDYYIWRDPVDGHEPTNWGSYFSGSAWQYDETSGQYYLHLFAVKQPDLNWENEAVRHSVYD
IMNWWADLGVDGFRMDVINLISKPAVYKDVPTAPGMQYGDVEPVVANGHRMHEFLQEMHQAV
MAKHDLVTVGETPGATTDDAKKYANLEQTELNMVFEFEHVGLDGNDNPALGKWSDKKVSLPE
LRDNLVKWQTQLNGKAWNSLYWNNHDQPRVVSRFGNDDPKYRVVSAKMLATMLHCLQGTPYI
YAGEELGMTNTTFNSLSDYRDLESINAYHQLVDEEHLVDGKTMSRYLAIHSRDNARTPMQWD
DSKNAGFSDAEPWIAVNPNYSEINAKAALADPSSVFYHYQKLIQMRHDLPVMTEGKFALVNG
NELDEQVFAYTRDDGETTLLVVANFTKETIKREYAAEQGKLLLSNYDDDMSETLRPYEAKVY
EFSSKR.
SEQ ID NO: 10 (L. fermentum isomaltase) V202
MIYTPKWWWQNSVVYQVYPRSFQDSNHDGIGDLKGIISRLDYIKKLGADIIWLNPIYRSPNV
DNGYDISDYRAIDPTFGSLTDFKELLAKAHELGLKIMMDLVVNHSSDENEWFKQSRQGKENP
YRDYYIWKDPVDGHEPTNWGSYFSGSAWQYDETSGQYYLHLFAVKQPDLNWENPAVRHSVYD
IMNWWADLGVDGFRMDVINLISKPAVYKDVPTAPGMQYGDVEPVVANGHRMHEFLQEMHQAV
MAKHDLVTVGETPGATTDDAKKYANLEQTELNMVFEFEHVGLDGNDNPALGKWSDKKVSLPE
LRDNLVKWQTQLNGKAWNSLYWNNHDQPRVVSRFGNDDPKYRVVSAKMLATMLHCLQGTPYI
YAGEELGMTNTTFNSLSDYRDLESINAYHQLVDEEHLVDGKTMSRYLAIHSRDNARTPMQWD
DSKNAGFSDAEPWIAVNPNYSEINAKAALADPSSVFYHYQKLIQMRHDLPVMTEGKFALVNG
NDLDEQVFAYTRDDGETTLLVVANFTKETIKREYDAGQGKLLLSNYEDDMGETLRPYEAKVY
ELSSKR.
SEQ ID NO: 11 (L. fermentum isomaltase) V210
MIYTPKWWWQNSVVYQVYPRSFQDSNHDGIGDLKGIISRLDYIKKLGADIIWLNPIYRSPNV
DNGYDISDYRAIDPTFGSLTDFKELLTKAHELGLKIMMDLVVNHSSDENEWFIQSRQGKENP
YRDYYIWKDPVDGHEPTNWGSYFSGSAWQYDETSGQYYLHLFAVKQPDLNWENEAVRHSVYE
IMNWWADLGVDGFRMDVINLISKPAVYKDVPTAPGMQYGDVEPVVANGHRMHEFLQEMHQAV
MAKHDLVTVGETPGATTDDAKKYANLEQTELNMVFEFEHVGLDGNDNPALGKWSDKKVSLPE
LRDNLVKWQTQLNGKAWNSLYWNNHDQPRVVSRFGNDDPKYRVVSAKMLATMLHCLQGTPYI
YAGEELGMTNTTFNSLSDYRDLESINAYHQLVDEEHLVDGKTMSRYLAIHSRDNARTPMQWD
DSKNAGFSDAEPWIAVNPNYAEINAKAALADPSSVFYHYQKLIQMRHDLPVMTEGKFALVNG
NDLDEQVFAYTRDDGETTLLVVANFTKETIKREYDAGQGKLLLSNYEDDMGETLRPYEAKVY
EFSSKR.
SEQ ID NO: 12 (L. fermentum isomaltase) V237
MIYTPKWWWQNSVVYQVYPRSFQDSNHDGIGDLKGIISRLDYIKKLGADIIWLNPIYRSPNV
DNGYDISDYRAIDPTFGSLADFKELLTKAHELGLKIMMDLVVNHSSDENEWFKQSRQGKENP
YRDYYIWKDPVDGHEPTNWGSYFSGSAWQYDETSGQYYLHLFAVKQPDLNWENPAVRHSVYD
IMNWWADLGVDGFRMDVINLISKPAVYKDVPTAPGMQYGDVEPVVANGHRMHEFLQEMHQAV
MAKHDLVTVGETPGATTDDAKKYANLEQTELNMVFEFEHVGLDGNDNPALGKWSDKKVSLPE
LRDNLVKWQTQLNGKAWNSLYWNNHDQPRVVSRFGNDDPKYRVVSAKMLATMLHCLQGTPYI
YAGEELGMTNTTFNSLSDYRDLESINAYHQLVDEEHLVDGKTMSRYLAIHSRDNARTPMQWD
DSKNAGESDAEPWIAVNPNYSEINAKAALADPSSVFYHYQKLIQMRHDLPVMTEGKFALVNG
NDLDDQVFAYTRDDGETTLLVVANFTKETIKREYDAGQGKLLLSNYEDDMGETLRPYEAKVY
EFSSKR.
SEQ ID NO: 13 (wild type S. cerevisiae invertase, AFN08663.1):
MLLQAFLFLLAGFAAKISASMTNETSDRPLVHFTPNKGWMNDPNGLWYDEKDAKWHLYFQYN
PNDTVWGTPLFWGHATSDDLTHWEDEPIAIAPKRNDSGAFSGSMVVDYNNTSGFFNDTIDPR
QRCVAIWTYNTPESEEQYISYSLDGGYTFTEYQKNPVLAANSTQFRDPKVEWYEPSQKWIMT
AAKSQDYKIEIYSSDDLKSWKLESAFANEGFLGYQYECPGLIEVPTEQDPSKSYWVMFISIN
PGAPAGGSFNQYFVGSFNGTHFEAFDNQSRVVDFGKDYYALQTFFNTDPTYGSALGIAWASN
WEYSAFVPTNPWRSSMSLVRKFSLNTEYQANPETELINLKAEPILNISNAGPWSRFATNTTL
TKANSYNVDLSNSTGTLEFELVYAVNTTQTISKSVFPDLSLWFKGLEDPEEYLRMGFEASAS
SFFLDRGNSKVKFVKENPYFTNRMSVNNQPFKSENDLSYYKVYGLLDQNILELYENDGDVVS
TNTYFMTTGNALGSVNMTTGVDNLFYIDKFQVREVK
SEQ ID NO: 14 (wild type S. cerevisiae invertase without signal
sequence):
SMTNETSDRPLVHFTPNKGWMNDPNGLWYDEKDAKWHLYFQYNPNDTVWGTPLFWGHATSDD
LTHWEDEPIAIAPKRNDSGAFSGSMVVDYNNTSGFFNDTIDPRQRCVAIWTYNTPESEEQYI
SYSLDGGYTFTEYQKNPVLAANSTQFRDPKVFWYEPSQKWIMTAAKSQDYKIEIYSSDDLKS
WKLESAFANEGFLGYQYECPGLIEVPTEQDPSKSYWVMFISINPGAPAGGSFNQYFVGSFNG
THFEAFDNQSRVVDFGKDYYALQTFFNTDPTYGSALGIAWASNWEYSAFVPTNPWRSSMSLV
RKFSLNTEYQANPETELINLKAEPILNISNAGPWSRFATNTTLTKANSYNVDLSNSTGTLEF
ELVYAVNTTQTISKSVFPDLSLWFKGLEDPEEYLRMGFEASASSFFLDRGNSKVKFVKENPY
FTNRMSVNNQPFKSENDLSYYKVYGLLDQNILELYFNDGDVVSTNTYFMTTGNALGSVNMTT
GVDNLFYIDKFQVREVK
SEQ ID NO: 15 (wild type S. cerevisiae invertase DNA sequence):
ATGCTTTTGCAAGCTTTCCTTTTCCTTTTGGCTGGTTTTGCAGCCAAAATATCTGCATCAAT
GACAAACGAAACTAGCGATAGACCTTTGGTCCACTTCACACCCAACAAGGGCTGGATGAATG
ACCCAAATGGGTTGTGGTACGATGAAAAAGATGCCAAATGGCATCTGTACTTTCAATACAAC
CCAAATGACACCGTATGGGGTACGCCATTGTTTTGGGGCCATGCTACTTCCGATGATTTGAC
TCATTGGGAAGATGAACCCATTGCTATCGCTCCCAAGCGTAACGATTCAGGTGCTTTCTCTG
GCTCCATGGTGGTTGATTACAACAACACGAGTGGGTTTTTCAATGATACTATTGATCCAAGA
CAAAGATGCGTTGCGATTTGGACTTATAACACTCCTGAAAGTGAAGAGCAATACATTAGCTA
TTCTCTTGATGGTGGTTACACTTTTACTGAATACCAAAAGAACCCTGTTTTAGCTGCCAACT
CCACTCAATTCAGAGATCCAAAGGTGTTCTGGTATGAACCTTCTCAAAAATGGATTATGACG
GCTGCCAAATCACAAGACTACAAAATTGAAATTTACTCCTCTGATGACTTGAAGTCCTGGAA
GCTAGAATCTGCATTTGCTAATGAAGGTTTCTTAGGCTACCAATATGAATGTCCAGGTTTGA
TTGAAGTCCCAACTGAGCAAGATCCTTCCAAATCCTATTGGGTCATGTTTATTTCTATCAAT
CCAGGTGCACCTGCTGGCGGTTCCTTCAACCAATATTTTGTTGGATCCTTCAATGGTACTCA
TTTTGAAGCGTTTGACAATCAATCTAGAGTGGTAGATTTTGGTAAGGACTACTATGCCTTGC
AAACTTTCTTCAACACAGACCCAACGTACGGTTCAGCATTAGGTATTGCCTGGGCTTCAAAC
TGGGAGTACAGTGCCTTTGTCCCAACTAACCCATGGAGATCATCCATGTCTTTGGTCCGCAA
GTTTTCTTTGAACACTGAATATCAAGCTAATCCAGAGACTGAATTGATCAATTTGAAAGCCG
AACCAATATTGAACATTAGTAATGCTGGTCCCTGGTCTCGTTTTGCTACTAACACAACTCTA
ACTAAGGCCAATTCTTACAATGTCGATTTGAGCAACTCGACTGGTACCCTAGAGTTTGAGTT
GGTTTACGCTGTTAACACCACACAAACCATATCCAAATCCGTCTTTCCCGACTTATCACTTT
GGTTCAAGGGTTTAGAAGATCCTGAAGAATATTTAAGAATGGGTTTTGAAGCCAGTGCTTCT
TCCTTCTTTTTGGACCGTGGTAACTCTAAGGTCAAGTTTGTCAAGGAGAACCCATATTTCAC
AAACAGAATGTCTGTCAACAACCAACCATTCAAGTCTGAGAACGACCTAAGTTACTATAAAG
TGTACGGCCTACTGGATCAAAACATCTTGGAATTGTACTTCAACGATGGAGATGTGGTTTCT
ACAAATACCTACTTCATGACCACCGGTAACGCTCTAGGATCTGTGAACATGACCACTGGTGT
CGATAATTTGTTCTACATTGACAAGTTCCAAGTAAGGGAAGTAAAATAG
SEQ ID NO: 16 (wild type S. cerevisiae invertase with F172Y, N429A,
and P479A mutations and FAKS signal sequence in bold):
MRFPSIFTAVLFAASSALAAPVNTTTEDETAQIPAEAVIGYSDLEGDFDVAVLPFSNSTNNG
LLFINTTIASIAAKEEGVSLEKREAEASMTNETSDRPLVHFTPNKGWMNDPNGLWYDEKDAK
WHLYFQYNPNDTVWGTPLFWGHATSDDLTHWEDEPIAIAPKRNDSGAYSGSMVVDYNNTSGF
FNDTIDPRQRCVAIWTYNTPESEEQYISYSLDGGYTFTEYQKNPVLAANSTQFRDPKVFWYE
PSQKWIMTAAKSQDYKIEIYSSDDLKSWKLESAFANEGFLGYQYECPGLIEVPTEQDPSKSY
WVMFISINPGAPAGGSFNQYFVGSFNGTHFEAFDNQSRVVDFGKDYYALQTFFNTDPTYGSA
LGIAWASNWEYSAFVPTNPWRSSMSLVRKESLNTEYQANPETELINLKAEPILNISAAGPWS
RFATNTTLTKANSYNVDLSNSTGTLEFELVYAVNTTQTISKSVFADLSLWEKGLEDPEEYLR
MGFEASASSFFLDRGNSKVKFVKENPYFTNRMSVNNQPFKSENDLSYYKVYGLLDQNILELY
FNDGDVVSTNTYFMTTGNALGSVNMTTGVDNLFYIDKFQVREVK.
SEQ ID NO: 17 (wild type S. cerevisiae invertase with no signal
sequence but with F172Y, N429A, and P479A mutations relative to SEQ
ID NO: 16):
SMTNETSDRPLVHFTPNKGWMNDPNGLWYDEKDAKWHLYFQYNPNDTVWGTPLFWGHATSDD
LTHWEDEPIAIAPKRNDSGAYSGSMVVDYNNTSGFFNDTIDPRQRCVAIWTYNTPESEEQYI
SYSLDGGYTFTEYQKNPVLAANSTQFRDPKVFWYEPSQKWIMTAAKSQDYKIEIYSSDDLKS
WKLESAFANEGFLGYQYECPGLIEVPTEQDPSKSYWVMFISINPGAPAGGSFNQYFVGSFNG
THFEAFDNQSRVVDFGKDYYALQTFFNTDPTYGSALGIAWASNWEYSAFVPTNPWRSSMSLV
RKFSLNTEYQANPETELINLKAEPILNISAAGPWSRFATNTTLTKANSYNVDLSNSTGTLEF
ELVYAVNTTQTISKSVFADLSLWFKGLEDPEEYLRMGFEASASSFFLDRGNSKVKFVKENPY
FTNRMSVNNQPFKSENDLSYYKVYGLLDQNILELYENDGDVVSTNTYFMTTGNALGSVNMTT
GVDNLFYIDKFQVREVK
SEQ ID NO 18 (FAKS signal sequence):
MRFPSIFTAVLFAASSALAAPVNTTTEDETAQIPAEAVIGYSDLEGDFDVAVLPFSNSTNNG
LLFINTTIASIAAKEEGVSLEKREAEA
SEQ ID NO: 19 (Sc_S288C invertase with F172Y and N429A mutations
relative to SEQ ID NO: 16, with native signal sequence shown in bold):
MLLQAFLFLLAGFAAKISASMTNETSDRPLVHFTPNKGWMNDPNGLWYDEKDAKWHLYFQYN
PNDTVWGTPLFWGHATSDDLINWEDQPIAIAPKRNDSGAYSGSMVVDYNNTSGFFNDTIDPR
QRCVAIWTYNTPESEEQYISYSLDGGYTFTEYQKNPVLAANSTQFRDPKVFWYEPSQKWIMT
AAKSQDYKIEIYSSDDLKSWKLESAFANEGFLGYQYECPGLIEVPTEQDPSKSYWVMFISIN
PGAPAGGSFNQYFVGSFNGTHFEAFDNQSRVVDFGKDYYALQTFFNTDPTYGSALGIAWASN
WEYSAFVPTNPWRSSMSLVRKFSLNTEYQANPETELINLKAEPILNISAAGPWSRFATNTTL
TKANSYNVDLSNSTGTLEFELVYAVNTTQTISKSVFADLSLWFKGLEDPEEYLRMGFEVSAS
SFFLDRGNSKVKFVKENPYFTNRMSVNNQPFKSENDLSYYKVYGLLDQNILELYFNDGDVVS
TNTYFMTTGNALGSVNMTTGVDNLFYIDKFQVREVK
SEQ ID NO: 20 (Sc_S288C invertase with no signal sequence but with
F172Y and N429A mutations relative to SEQ ID NO:16):
SMTNETSDRPLVHFTPNKGWMNDPNGLWYDEKDAKWHLYFQYNPNDTVWGTPLFWGHATSDD
LTNWEDQPIAIAPKRNDSGAYSGSMVVDYNNTSGFFNDTIDPRQRCVAIWTYNTPESEEQYI
SYSLDGGYTFTEYQKNPVLAANSTQFRDPKVEWYEPSQKWIMTAAKSQDYKIEIYSSDDLKS
WKLESAFANEGFLGYQYECPGLIEVPTEQDPSKSYWVMFISINPGAPAGGSFNQYFVGSFNG
THFEAFDNQSRVVDFGKDYYALQTFFNTDPTYGSALGIAWASNWEYSAFVPTNPWRSSMSLV
RKFSLNTEYQANPETELINLKAEPILNISAAGPWSRFATNTTLTKANSYNVDLSNSTGTLEF
ELVYAVNTTQTISKSVFADLSLWFKGLEDPEEYLRMGFEVSASSFFLDRGNSKVKFVKENPY
FTNRMSVNNQPFKSENDLSYYKVYGLLDQNILELYFNDGDVVSTNTYFMTTGNALGSVNMTT
GVDNLFYIDKFQVREVK

What is claimed is:

1. A recombinant mutant isomaltase enzyme (e.g., a recombinant mutant Limosilactobacillus fermentum isomaltase enzyme), wherein the isomaltase comprises:
(i) increased activity at acidic pH (e.g., about pH 4.0 and 6.0) relative to a corresponding wild-type isomaltase;
(ii) increased activity at neutral pH (e.g., about pH 7.0) relative to a corresponding wild-type isomaltase;
(iii) increased stability at acidic pH (e.g., about pH 4) relative to a corresponding wild-type isomaltase;
(iv) increased thermal stability relative to a corresponding wild-type isomaltase;
(v) increased proteolytic stability in the presence of pancreatin and/or pepsin relative to a corresponding wild-type isomaltase; or
(vi) a combination of any of the foregoing features (i) to (v).

2. The isomaltase of claim 1, wherein the isomaltase comprises:
(a) a substitution of a residue at a position corresponding to position 115 of wild-type L. fermentum isomaltase;
(b) a substitution of a residue at a position corresponding to position 132 of wild-type L. fermentum isomaltase;
(c) a substitution of a residue at a position corresponding to position 226 of wild-type L. fermentum isomaltase;
(d) a substitution of a residue at a position corresponding to position 310 of wild-type L. fermentum isomaltase;
(e) a substitution of a residue at a position corresponding to position 366 of wild-type L. fermentum isomaltase;
(f) a substitution of a residue at a position corresponding to position 421 of wild-type L. fermentum isomaltase;
(g) a substitution of a residue at a position corresponding to position 444 of wild-type L. fermentum isomaltase;
(h) a substitution of a residue at a position corresponding to position 531 of wild-type L. fermentum isomaltase;
(i) a substitution of a residue at a position corresponding to position 560 of wild-type L. fermentum isomaltase;
(j) a substitution of a residue at a position corresponding to position 12 of wild-type L. fermentum isomaltase;
(k) a substitution of a residue at a position corresponding to position 82 of wild-type L. fermentum isomaltase;
(l) a substitution of a residue at a position corresponding to position 86 of wild-type L. fermentum isomaltase;
(m) a substitution of a residue at a position corresponding to position 89 of wild-type L. fermentum isomaltase;
(n) a substitution of a residue at a position corresponding to position 93 of wild-type L. fermentum isomaltase;
(o) a substitution of a residue at a position corresponding to position 122 of wild-type L. fermentum isomaltase;
(p) a substitution of a residue at a position corresponding to position 136 of wild-type L. fermentum isomaltase;
(q) a substitution of a residue at a position corresponding to position 157 of wild-type L. fermentum isomaltase;
(r) a substitution of a residue at a position corresponding to position 168 of wild-type L. fermentum isomaltase;
(s) a substitution of a residue at a position corresponding to position 178 of wild-type L. fermentum isomaltase;
(t) a substitution of a residue at a position corresponding to position 182 of wild-type L. fermentum isomaltase;
(u) a substitution of a residue at a position corresponding to position 186 of wild-type L. fermentum isomaltase;
(v) a substitution of a residue at a position corresponding to position 211 of wild-type L. fermentum isomaltase;
(w) a substitution of a residue at a position corresponding to position 246 of wild-type L. fermentum isomaltase;
(x) a substitution of a residue at a position corresponding to position 269 of wild-type L. fermentum isomaltase;
(y) a substitution of a residue at a position corresponding to position 275 of wild-type L. fermentum isomaltase;
(z) a substitution of a residue at a position corresponding to position 309 of wild-type L. fermentum isomaltase;
(aa) a substitution of a residue at a position corresponding to position 394 of wild-type L. fermentum isomaltase;
(bb) a substitution of a residue at a position corresponding to position 455 of wild-type L. fermentum isomaltase;
(cc) a substitution of a residue at a position corresponding to position 486 of wild-type L. fermentum isomaltase;
(dd) a substitution of a residue at a position corresponding to position 498 of wild-type L. fermentum isomaltase;
(ee) a substitution of a residue at a position corresponding to position 501 of wild-type L. fermentum isomaltase;
(ff) a substitution of a residue at a position corresponding to position 502 of wild-type L. fermentum isomaltase;
(gg) a substitution of a residue at a position corresponding to position 514 of wild-type L. fermentum isomaltase;
(hh) a substitution of a residue at a position corresponding to position 524 of wild-type L. fermentum isomaltase;
(ii) a substitution of a residue at a position corresponding to position 533 of wild-type L. fermentum isomaltase;
(jj) a substitution of a residue at a position corresponding to position 535 of wild-type L. fermentum isomaltase;
(kk) a substitution of a residue at a position corresponding to position 543 of wild-type L. fermentum isomaltase;
(11) a substitution of a residue at a position corresponding to position 554 of wild-type L. fermentum isomaltase;
(mm) a substitution of a residue at a position corresponding to position 556 of wild-type L. fermentum isomaltase;
or a combination of any of the foregoing substitutions, wherein the wild-type L. fermentum isomaltase comprises the amino acid sequence of SEQ ID NO:1.

3. The isomaltase of claim 2, wherein, in the isomaltase:
(a) the residue at a position corresponding to position 115 of wild-type L. fermentum isomaltase is substituted by I;
(b) the residue at a position corresponding to position 132 of wild-type L. fermentum isomaltase is substituted by K;
(c) the residue at a position corresponding to position 226 of wild-type L. fermentum isomaltase is substituted by S;
(d) the residue at a position corresponding to position 310 of wild-type L. fermentum isomaltase is substituted by A;
(e) the residue at a position corresponding to position 366 of wild-type L. fermentum isomaltase is substituted by M;
(f) the residue at a position corresponding to position 421 of wild-type L. fermentum isomaltase is substituted by A;
(g) the residue at a position corresponding to position 444 of wild-type L. fermentum isomaltase is substituted by G;
(h) the residue at a position corresponding to position 531 of wild-type L. fermentum isomaltase is substituted by D;
(i) the residue at a position corresponding to position 560 of wild-type L. fermentum isomaltase is substituted by V;
(j) the residue at a position corresponding to position 12 of wild-type L. fermentum isomaltase is substituted by A;
(k) the residue at a position corresponding to position 82 of wild-type L. fermentum isomaltase is substituted by A;
(l) the residue at a position corresponding to position 86 of wild-type L. fermentum isomaltase is substituted by Q;
(m) the residue at a position corresponding to position 89 of wild-type L. fermentum isomaltase is substituted by A;
(n) the residue at a position corresponding to position 93 of wild-type L. fermentum isomaltase is substituted by K;
(o)
(p) the residue at a position corresponding to position 122 of wild-type L. fermentum isomaltase is substituted by D;
(q) the residue at a position corresponding to position 136 of wild-type L. fermentum isomaltase is substituted by N;
(r) the residue at a position corresponding to position 157 of wild-type L. fermentum isomaltase is substituted by A;
(s) the residue at a position corresponding to position 168 of wild-type L. fermentum isomaltase is substituted by K;
(t) the residue at a position corresponding to position 178 of wild-type L. fermentum isomaltase is substituted by D;
(u) the residue at a position corresponding to position 178 of wild-type L. fermentum isomaltase is substituted by P;
(v) the residue at a position corresponding to position 182 of wild-type L. fermentum isomaltase is substituted by R;
(w) the residue at a position corresponding to position 186 of wild-type L. fermentum isomaltase is substituted by E;
(x) the residue at a position corresponding to position 211 of wild-type L. fermentum isomaltase is substituted by E;
(y) the residue at a position corresponding to position 246 of wild-type L. fermentum isomaltase is substituted by R;
(z) the residue at a position corresponding to position 269 of wild-type L. fermentum isomaltase is substituted by R;
(aa) the residue at a position corresponding to position 275 of wild-type L. fermentum isomaltase is substituted by D;
(bb) the residue at a position corresponding to position 309 of wild-type L. fermentum isomaltase is substituted by R;
(cc) the residue at a position corresponding to position 394 of wild-type L. fermentum isomaltase is substituted by I;
(dd) the residue at a position corresponding to position 455 of wild-type L. fermentum isomaltase is substituted by A;
(ee) the residue at a position corresponding to position 486 of wild-type L. fermentum isomaltase is substituted by I;
(ff) the residue at a position corresponding to position 498 of wild-type L. fermentum isomaltase is substituted by N;
(gg) the residue at a position corresponding to position 498 of wild-type L. fermentum isomaltase is substituted by D;
(hh) the residue at a position corresponding to position 501 of wild-type L. fermentum isomaltase is substituted by D;
(ii) the residue at a position corresponding to position 502 of wild-type L. fermentum isomaltase is substituted by E;
(jj) the residue at a position corresponding to position 514 of wild-type L. fermentum isomaltase is substituted by K;
(kk) the residue at a position corresponding to position 524 of wild-type L. fermentum isomaltase is substituted by Q;
(ll) the residue at a position corresponding to position 533 of wild-type L. fermentum isomaltase is substituted by E;
(mm) the residue at a position corresponding to position 535 of wild-type L. fermentum isomaltase is substituted by S;
(nn) the residue at a position corresponding to position 543 of wild-type L. fermentum isomaltase is substituted by D;
(oo) the residue at a position corresponding to position 543 of wild-type L. fermentum isomaltase is substituted by N;
(pp) the residue at a position corresponding to position 547 of wild-type L. fermentum isomaltase is substituted by S;
(qq) the residue at a position corresponding to position 551 of wild-type L. fermentum isomaltase is substituted by N;
(rr) the residue at a position corresponding to position 554 of wild-type L. fermentum isomaltase is substituted by Q;
(ss) the residue at a position corresponding to position 556 of wild-type L. fermentum isomaltase is substituted by I;
(tt) the residue at a position corresponding to position 560 of wild-type L. fermentum isomaltase is substituted by I;
(uu) the residue at a position corresponding to position 560 of wild-type L. fermentum isomaltase is substituted by L;
or the isomaltase comprises a combination of any of the foregoing substitutions.

4. The isomaltase of any one of claims 1-3, wherein the isomaltase comprises:
a substitution of a K residue at a position corresponding to position 115 of wild-type L. fermentum isomaltase (SEQ ID NO: 1); a substitution of a R residue at a position corresponding to position 132 of wild-type L. fermentum isomaltase (SEQ ID NO: 1); a substitution of a D residue at a position corresponding to position 226 of wild-type L. fermentum isomaltase (SEQ ID NO: 1); a substitution of an E residue at a position corresponding to position 310 of wild-type L. fermentum isomaltase (SEQ ID NO: 1); a substitution of a L residue at a position corresponding to position 366 of wild-type L. fermentum isomaltase (SEQ ID NO: 1); a substitution of a I residue at a position corresponding to position 421 of wild-type L. fermentum isomaltase (SEQ ID NO: 1); a substitution of an A residue at a position corresponding to position 444 of wild-type L. fermentum isomaltase (SEQ ID NO: 1); a substitution of an A residue at a position corresponding to position 531 of wild-type L. fermentum isomaltase (SEQ ID NO: 1); a substitution of a F residue at a position corresponding to position 560 of wild-type L. fermentum isomaltase (SEQ ID NO: 1); a substitution of a T residue at a position corresponding to position 82 of wild-type L. fermentum isomaltase (SEQ ID NO: 1); a substitution of an E residue at a position corresponding to position 86 of wild-type L. fermentum isomaltase (SEQ ID NO: 1); a substitution of a T residue at a position corresponding to position 89 of wild-type L. fermentum isomaltase (SEQ ID NO: 1); a substitution of an E residue at a position corresponding to position 93 of wild-type L. fermentum isomaltase (SEQ ID NO: 1); a substitution of an E residue at a position corresponding to position 122 of wild-type L. fermentum isomaltase (SEQ ID NO: 1); a substitution of a D residue at a position corresponding to position 136 of wild-type L. fermentum isomaltase (SEQ ID NO: 1); a substitution of a T residue at a position corresponding to position 157 of wild-type L. fermentum isomaltase (SEQ ID NO: 1); a substitution of a V residue at a position corresponding to position 168 of wild-type L. fermentum isomaltase (SEQ ID NO: 1); a substitution of an E residue at a position corresponding to position 178 of wild-type L. fermentum isomaltase (SEQ ID NO: 1); a substitution of a H residue at a position corresponding to position 182 of wild-type L. fermentum isomaltase (SEQ ID NO: 1); a substitution of a D residue at a position corresponding to position 186 of wild-type L. fermentum isomaltase (SEQ ID NO: 1); a substitution of an A residue at a position corresponding to position 211 of wild-type L. fermentum isomaltase (SEQ ID NO: 1); a substitution of a Q residue at a position corresponding to position 246 of wild-type L. fermentum isomaltase (SEQ ID NO: 1); a substitution of a K residue at a position corresponding to position 269 of wild-type L. fermentum isomaltase (SEQ ID NO: 1); a substitution of an E residue at a position corresponding to position 275 of wild-type L. fermentum isomaltase (SEQ ID NO: 1); a substitution of a P residue at a position corresponding to position 309 of wild-type L. fermentum isomaltase (SEQ ID NO: 1); a substitution of a L residue at a position corresponding to position 394 of wild-type L. fermentum isomaltase (SEQ ID NO: 1); a substitution of a S residue at a position corresponding to position 455 of wild-type L. fermentum isomaltase (SEQ ID NO: 1); a substitution of a M residue at a position corresponding to position 486 of wild-type L. fermentum isomaltase (SEQ ID NO: 1); a substitution of a E residue at a position corresponding to position 498 of wild-type L. fermentum isomaltase (SEQ ID NO: 1); a substitution of an E residue at a position corresponding to position 501 of wild-type L. fermentum isomaltase (SEQ ID NO: 1); a substitution of a Q residue at a position corresponding to position 502 of wild-type L. fermentum isomaltase (SEQ ID NO: 1); a substitution of a T residue at a position corresponding to position 514 of wild-type L. fermentum isomaltase (SEQ ID NO: 1); a substitution of an E residue at a position corresponding to position 524 of wild-type L. fermentum isomaltase (SEQ ID NO: 1); a substitution of a G residue at a position corresponding to position 533 of wild-type L. fermentum isomaltase (SEQ ID NO: 1); a substitution of a G residue at a position corresponding to position 535 of wild-type L. fermentum isomaltase (SEQ ID NO: 1); a substitution of an E residue at a position corresponding to position 543 of wild-type L. fermentum isomaltase (SEQ ID NO: 1); a substitution of a G residue at a position corresponding to position 547 of wild-type L. fermentum isomaltase (SEQ ID NO: 1); a substitution of a R residue at a position corresponding to position 551 of wild-type L. fermentum isomaltase (SEQ ID NO: 1); a substitution of an E residue at a position corresponding to position 554 of wild-type L. fermentum isomaltase (SEQ ID NO: 1); a substitution of a K residue at a position corresponding to position 556 of wild-type L. fermentum isomaltase (SEQ ID NO: 1); or a combination of any of the foregoing substitutions.

5. The isomaltase of any one of claims 1-3, wherein the isomaltase comprises:
(a) the K residue at a position corresponding to position 115 of wild-type L. fermentum isomaltase is substituted by I (K115I);
(b) the R residue at a position corresponding to position 132 of wild-type L. fermentum isomaltase is substituted by K (R132K);
(c) the D residue at a position corresponding to position 226 of wild-type L. fermentum isomaltase is substituted by S (D226S);
(d) the E residue at a position corresponding to position 310 of wild-type L. fermentum isomaltase is substituted by A (E310A);
(e) the L residue at a position corresponding to position 366 of wild-type L. fermentum isomaltase is substituted by M (L366M);
(f) the I residue at a position corresponding to position 421 of wild-type L. fermentum isomaltase is substituted by A (I421A);
(g) the A residue at a position corresponding to position 444 of wild-type L. fermentum isomaltase is substituted by G (A444G);
(h) the A residue at a position corresponding to position 531 of wild-type L. fermentum isomaltase is substituted by D (A531D);
(i) the F residue at a position corresponding to position 560 of wild-type L. fermentum isomaltase is substituted by V (F560V);
(j) the S residue at a position corresponding to position 12 of wild-type L. fermentum isomaltase is substituted by A (S12A);
(k) the T residue at a position corresponding to position 82 of wild-type L. fermentum isomaltase is substituted by A (T82A);
(l) the E residue at a position corresponding to position 86 of wild-type L. fermentum isomaltase is substituted by Q (E86Q);
(m) the T residue at a position corresponding to position 89 of wild-type L. fermentum isomaltase is substituted by A (T89A);
(n) the E residue at a position corresponding to position 93 of wild-type L. fermentum isomaltase is substituted by K (E93K);
(o) the E residue at a position corresponding to position 122 of wild-type L. fermentum isomaltase is substituted by D (E122D);
(p) the D residue at a position corresponding to position 136 of wild-type L. fermentum isomaltase is substituted by N (D136N);
(q) the T residue at a position corresponding to position 157 of wild-type L. fermentum isomaltase is substituted by A (T157A);
(r) the V residue at a position corresponding to position 168 of wild-type L. fermentum isomaltase is substituted by K (V168K);
(s) the E residue at a position corresponding to position 178 of wild-type L. fermentum isomaltase is substituted by D (E178D);
(t) the E residue at a position corresponding to position 178 of wild-type L. fermentum isomaltase is substituted by P (E178P);
(u) the H residue at a position corresponding to position 182 of wild-type L. fermentum isomaltase is substituted by R (H182R);
(v) the D residue at a position corresponding to position 186 of wild-type L. fermentum isomaltase is substituted by E (D186E);
(w) the A residue at a position corresponding to position 211 of wild-type L. fermentum isomaltase is substituted by E (A211E);
(x) the Q residue at a position corresponding to position 246 of wild-type L. fermentum isomaltase is substituted by R (Q246R);
(y) the K residue at a position corresponding to position 269 of wild-type L. fermentum isomaltase is substituted by R (K269R);
(z) the E residue at a position corresponding to position 275 of wild-type L. fermentum isomaltase is substituted by D (E275D);
(aa) the P residue at a position corresponding to position 309 of wild-type L. fermentum isomaltase is substituted by R (P309R);
(bb) the L residue at a position corresponding to position 394 of wild-type L. fermentum isomaltase is substituted by I (L394I);
(cc) the S residue at a position corresponding to position 455 of wild-type L. fermentum isomaltase is substituted by A (S455A);
(dd) the M residue at a position corresponding to position 486 of wild-type L. fermentum isomaltase is substituted by I (M486I);
(ee) the E residue at a position corresponding to position 498 of wild-type L. fermentum isomaltase is substituted by N (E498N);
(ff) the E residue at a position corresponding to position 498 of wild-type L. fermentum isomaltase is substituted by D (E498D);
(gg) the E residue at a position corresponding to position 501 of wild-type L. fermentum isomaltase is substituted by D (E501D);
(hh) the Q residue at a position corresponding to position 502 of wild-type L. fermentum isomaltase is substituted by E (Q502E);
(ii) the T residue at a position corresponding to position 514 of wild-type L. fermentum isomaltase is substituted by K (T514K);
(jj) the E residue at a position corresponding to position 524 of wild-type L. fermentum isomaltase is substituted by Q (E524Q);
(kk) the G residue at a position corresponding to position 533 of wild-type L. fermentum isomaltase is substituted by E (G533E);
(ll) the G residue at a position corresponding to position 535 of wild-type L. fermentum isomaltase is substituted by S (G535S);
(mm) the E residue at a position corresponding to position 543 of wild-type L. fermentum isomaltase is substituted by D (E543D);
(nn) the E residue at a position corresponding to position 543 of wild-type L. fermentum isomaltase is substituted by N (E543N);
(oo) the G residue at a position corresponding to position 547 of wild-type L. fermentum isomaltase is substituted by S (G547S);
(pp) the R residue at a position corresponding to position 551 of wild-type L. fermentum isomaltase is substituted by N (R551N);
(qq) the E residue at a position corresponding to position 554 of wild-type L. fermentum isomaltase is substituted by Q (E554Q);
(rr) the K residue at a position corresponding to position 556 of wild-type L. fermentum isomaltase is substituted by I (K556I);
(ss) the F residue at a position corresponding to position 560 of wild-type L. fermentum isomaltase is substituted by I (F560I);
(tt) the F residue at a position corresponding to position 560 of wild-type L. fermentum isomaltase is substituted by L (F560L);
or the isomaltase comprises a combination of any of the foregoing substitutions.

6. The isomaltase of any one of claims 1-5, wherein the isomaltase comprises one, two, three, four, five, six, seven, eight, nine, ten, or more than ten mutations relative to the corresponding wild-type L. fermentum isomaltase (SEQ ID NO:1).

7. The isomaltase of any one of claims 1-6, wherein the isomaltase comprises an amino acid sequence comprising:
(a) at least one substitution relative to SEQ ID NO: 1 selected from the group consisting of K115I, R132K, A444G, and A531D;
(b) the amino acid substitutions K115I, R132K, A444G, and A531D relative to SEQ ID NO: 1;
(c) at least one substitution selected from the group consisting of K115I, D226S, I421A, A444G, and A531D relative to SEQ ID NO: 1;
(d) the amino acid substitutions K115I, D226S, I421A, A444G, and A531D, and optionally further comprises either F560V or F560L relative to SEQ ID NO: 1;
(e) the amino acid substitutions K115I, D226S, I421A, A444G, and A531D, and optionally further comprises one or more substitutions selected from the group consisting of T89A, E93K, E122D, R132K, H182R, A211E, E275D, E310A, L366M, and E524Q relative to SEQ ID NO. 1;
(f) an amino acid sequence of SEQ. ID NO: 1 with at least the amino acid substitutions K115I, D226S, I421A, A444G, and A531D; or
(g) an amino acid sequence of SEQ. ID NO: 1 with at least the amino acid substitutions K115I, D226S, I421A, A444G, A53 iD, and either F560V or F560L.

8. The isomaltase of claim 3 or 4, wherein the isomaltase comprises:
(a) the K115I, R132K, D226S, E310A, L366M, I421A, A444G, A531D, and F560V substitutions;
(b) the E93K, K115I, R132K, D226S, E310A, L366M, I421A, A444G, A531D, and F560L substitutions;
(c) the E93K, K115I, R132K, D226S, E310A, I421A, A444G, E524Q, A531D, and F560L substitutions;
(d) the E93K, K115I, R132K, D226S, E310A, I421A, A444G, A531D, and F560V substitutions;
(e) the E93K, K115I, R132K, D226S, L366M, I421A, A444G, E524Q, A531D, and F560L substitutions;
(f) the E93K, K115I, R132K, D226S, L366M, I421A, A444G, A531D, and F560V substitutions;
(g) the E93K, K115I, R132K, D226S, I421A, A444G, E524Q, A531D, and F560V substitutions;
(h) the K115I, R132K, D226S, E310A, L366M, I421A, A444G, E524Q, A531D, and F560L substitutions;
(i) the K115I, R132K, D226S, E310A, I421A, A444G, E524Q, A531D, and F560V substitutions;
(j) the K115I, R132K, D226S, L366M, I421A, A444G, E524Q, A531D, and F560V substitutions;
(k) the E93K, K115I, R132K, A211E, D226S, I421A, A444G, E524Q, A531D, and F560V substitutions;
(l) the E93K, K115I, R132K, D226S, L366M, I421A, A444G, E524Q, A531D, and F560V substitutions;
(m) the E93K, K115I, R132K, D226S, E275D, I421A, A444G, E524Q, A531D, and F560V substitutions;
(n) the T89A, E93K, K115I, R132K, D226S, I421A, A444G, E524Q, A531D, and F560V substitutions;
(o) the E93K, K115I, R132K, H182R, D226S, I421A, A444G, E524Q, A531D, and F560V substitutions;
(p) the E93K, K115I, E122D, R132K, D226S, I421A, A444G, E524Q, A531D, and F560V substitutions;
(q) the E93K, K115I, D226S, I421A, A444G, E524Q, A531D, and F560V substitutions;
(r) the E93K, K115I, D226S, I421A, A444G, E524Q, A531D, and F560L substitutions;
(s) the K115I, A211E, D226S, I421A, A444G, A531D, and F560V substitutions;
(t) the K115I, D226S, L366M, I421A, A444G, E524Q, A531D, and F560L substitutions;
(u) the K115I, A211E, D226S, I421A, A444G, E524Q, A531D, and F560L substitutions;
(v) the K115I, R132K, D226S, E310A, L366M, I421A, A444G, A531D, and F560L substitutions;
(w) the E93K, K115I, D226S, E310A, I421A, A444G, A531D, and F560L substitutions;
(x) the K115I, D226S, E310A, I421A, A444G, A531D, and F560V substitutions;
(y) the E93K, K115I, D226S, L366M, I421A, A444G, A531D, and F560L substitutions;
(z) the K115I, D226S, I421A, A444G, E524Q, A531D, and F560V substitutions;
(aa) the K115I, D226S, E310A, I421A, A444G, E524Q, A531D, and F560L substitutions;
(bb) the K115I, A211E, D226S, L366M, I421A, A444G, A531D, and F560L substitutions;
(cc) the K115I, A211E, D226S, E310A, I421A, A444G, A531D, and F560L substitutions;
(dd) the E93K, K115I, D226S, I421A, A444G, A531D, and F560V substitutions;
(ee) the E93K, K115I, A211E, D226S, I421A, A444G, A531D, and F560L substitutions; or
(ff) the K115I, D226S, L366M, I421A, A444G, A531D, and F560V substitutions.

9. The isomaltase of claim 3 or 4, wherein the isomaltase comprises:
(a) the S12A, T89A, K115I, R132K, E275D, A444G, and A531D substitutions;
(b) the T89A, K115I, R132K, A444G, S455A, A531D, and F560I substitutions;
(c) the T89A, K115I, R132K, D226S, P309R, A444G, and A531D substitutions;
(d) the T82A, K115I, R132K, E275D, A444G, S455A, and A531D substitutions;
(e) the K115I, R132K, H182R, A444G, T514K, A531D, and E543N substitutions;
(f) the E93K, K115I, T157A, A444G, and A531D substitutions;
(g) the K115I, R132K, A211E, K269R, E275D, A444G, and A531D substitutions;
(h) the E86Q, K115I, R132K, A444G, M486I, A531D, and F560V substitutions;
(i) the K115I, R132K, T157A, D226S, A444G, E524Q, and A531D substitutions;
(j) the E93K, K115I, R132K, D136N, A444G, A53 iD, and K556I substitutions;
(k) the K115I, R132K, T157A, H182R, A444G, S455A, and A531D substitutions;
(l) the K115I, R132K, E275D, A444G, A531D, R551N, and F560V substitutions;
(m) the K115I, E275D, P309R, A444G, and A531D substitutions;
(n) the K115I, R132K, E178D, L366M, A444G, A531D, and F560I substitutions;
(o) the S12A, K115I, R132K, H182R, K269R, A444G, and A531D substitutions;
(p) the T89A, E93K, K115I, R132K, H182R, A444G, and A531D substitutions;
(q) the K115I, A211E, L366M, A444G, and A531D substitutions;
(r) the T82A, K115I, R132K, A444G, Q502E, A531D, and F560L substitutions;
(s) the S12A, K115I, R132K, E178D, E310A, A444G, and A531D substitutions;
(t) the K115I, R132K, Q246R, A444G, A531D, G535S, and K556I substitutions;
(u) the E93K, K115I, R132K, E275D, A444G, A531D, and F560I substitutions;
(v) the T82A, E93K, K115I, R132K, L366M, A444G, and A531D substitutions;
(w) the E93K, K115I, R132K, V168K, A444G, M486I, and A531D substitutions;
(x) the S12A, T82A, K115I, R132K, and A531D substitutions;
(y) the E86Q, K115I, R132K, E275D, L366M, A444G, and A531D substitutions;
(z) the E93K, K115I, R132K, A444G, and G535S substitutions;
(aa) the K115I, R132K, D226S, I421A, A444G, A531D, and F560L substitutions;
(bb) the S12A, E93K, K115I, R132K, A444G, S455A, and A531D substitutions;
(cc) the K115I, R132K, K269R, A444G, A531D, E554Q, and K556I substitutions;
(dd) the K115I, E122D, R132K, E310A, A444G, A531D, and E543N substitutions;
(ee) the S12A, K115I, R132K, D136N, A444G, A531D, and G535S substitutions;
(ff) the K115I, R132K, E178D, E498N, and A531D substitutions;
(gg) the K115I, R132K, K269R, A444G, and S455A substitutions;
(hh) the T82A, K115I, R132K, D226S, L394I, A444G, and A531D substitutions;
(ii) the K115I, R132K, S455A, A531D, and F560L substitutions;
(jj) the K115I, R132K, E310A, A444G, Q502E, T514K, and A531D substitutions;
(kk) the S12A, K115I, R132K, L366M, and A444G substitutions;
(ll) the T82A, T89A, K115I, R132K, and A444G substitutions;
(mm) the K115I, R132K, T157A, A444G, A531D, G535S, and F560L substitutions;
(nn) the T82A, K115I, R132K, Q246R, A444G, A531D, and E543N substitutions;
(oo) the E93K, K115I, R132K, A211E, A444G, T514K, and A531D substitutions; or
(pp) the K115I, R132K, E178D, A211E, A444G, A531D, and F560L substitutions.

10. The isomaltase of any one of claims 1-9, wherein the isomaltase comprises the amino acid sequence of any one of SEQ ID NOs: 7, 1-6, and 8-12, or an amino acid sequence that has at least 85%, 90%, 95%, 96%, 97%, 98%, or 99% sequence identity to any one of SEQ ID NOs: 7, 1-6, and 8-12.

11. A recombinant mutant L. fermentum isomaltase enzyme comprising a substitution, or combination of substitutions listed in TABLE 1.

12. The isomaltase of any one of claims 1-11, wherein the isomaltase has a specific activity at about pH 4 of at least 25, 30, 35, 40, 45, 50, 55, 60, 65, 70, 75, or 80 μmol isomaltose consumed per minute per milligram of isomaltase.

13. The isomaltase of any one of claims 1-11, wherein the isomaltase has at least 1 fold, 1.5 fold, 2 fold, 2.5 fold, or 3 fold higher activity at about pH 4, compared to a corresponding wild-type isomaltase.

14. The isomaltase of any one of claims 1-11, wherein the isomaltase has a specific activity at about pH 6.0 of at least 30, 35, 40, 45, 50, 55, 60, 65, 70, 75, 80, 85, 90, 95, 100, 105, 110, or 115 μmol isomaltose consumed per minute per milligram of isomaltase.

15. The isomaltase of any one of claims 1-11, wherein the isomaltase has at least 1 fold, 1.5 fold, 2 fold, 2.5 fold, or 3 fold higher activity at about pH 6.0, compared to a corresponding wild-type isomaltase.

16. The isomaltase of any one of claims 1-11, wherein the isomaltase has a specific activity at about pH 7.0 of at least 25, 30, 35, 40, 45, 50, 55, 60, 65, 70, 75, 80, 85, 90, or 95, μmol isomaltose consumed per minute per milligram of isomaltase.

17. The isomaltase of any one of claims 1-11, wherein the isomaltase has at least 1 fold, 1.5 fold, 2 fold, 2.5 fold, or 3 fold higher activity at about pH 7.0, compared to a corresponding wild-type isomaltase.

18. The isomaltase of any one of claims 1-17, wherein the isomaltase has a Tm of at least 50, 51, 52, 53, 54, 55, 56° C.

19. The isomaltase of any one of claims 1-17, wherein the isomaltase has a Tm that is at least 1, 2, 3, 4, 5, 6, 7, 8, 9, or 10° C. higher than a corresponding wild-type isomaltase.

20. The isomaltase of any one of claims 1-19, wherein the isomaltase has higher stability in the presence of pancreatin or pepsin compared to a corresponding wild-type isomaltase.

21. A nucleic acid encoding the isomaltase of any one of claims 1-20.

22. An expression vector comprising the nucleic acid of claim 21.

23. A cell comprising the expression vector of claim 22.

24. The cell of claim 23, wherein the cell is an E. coli or a S. cerevisiae cell.

25. A method of producing a recombinant mutant L. fermentum isomaltase enzyme, the method comprising
a) growing the cell of claim 23 or 24 under conditions so that the host cell expresses the isomaltase, and
b) purifying the isomaltase.

26. A pharmaceutical composition comprising the isomaltase of any one of claims 1-20, and a pharmaceutically acceptable carrier and/or an excipient.

27. A pharmaceutical composition comprising an isomaltase enzyme of any one of claims 1-20, a separate invertase enzyme, and a pharmaceutically acceptable carrier and/or an excipient.

28. The pharmaceutical composition of claim 27, wherein the isomaltase is spray-dried.

29. The pharmaceutical composition of claim 27 or 28, wherein the invertase is spray-dried.

30. The pharmaceutical composition of any one of claims 26-29, wherein the isomaltase is a microbial or fungal isomaltase, or a functional fragment or variant thereof.

31. The pharmaceutical composition of any one of claims 27-30, wherein the invertase is a microbial or fungal invertase, or a functional fragment or variant thereof.

32. The pharmaceutical composition of any one of claims 26-31, wherein the isomaltase is derived from L. fermentum.

33. The pharmaceutical composition of claim 32, wherein the isomaltase comprises a sequence of any one of SEQ ID NOs: 7, 1-6, and 8-12, or a functional fragment or variant thereof.

34. The pharmaceutical composition of claim 32, wherein the isomaltase is the recombinant mutant L. fermentum isomaltase enzyme of any one of claims 1-20.

35. The pharmaceutical composition of any one of claims 27-34, wherein the invertase is derived from Saccharomyces cerevisiae.

36. The pharmaceutical composition of claim 35, wherein the invertase comprises any one of SEQ ID NOs: 16 or 17, or a functional fragment or variant thereof.

37. The pharmaceutical composition of claim 35, wherein the invertase comprises any one of SEQ ID NOs: 13-14, or a functional fragment or variant thereof.

38. The pharmaceutical composition of claim 35, wherein the invertase comprises any one of SEQ ID NOs: 19 or 20, or a functional fragment or variant thereof.

39. The pharmaceutical composition of any one of claims 26-38, wherein the composition is formulated as an oral dosage form.

40. The pharmaceutical composition of claim 39, wherein the composition is a formulated as a powder, satchel, granulate, pellet, micropellet, tablet, or minitablet.

41. The pharmaceutical composition of claim 39, wherein the composition is formulated as a powder, satchel, or tablet.

42. The pharmaceutical composition of any one of claims 26-41, wherein the composition has a shelf-life at room temperature of at least 3 months, 6 months, 9 months, 12 months, 15 months, 18 months, 21 months, 24 months, 36 months, 48 months, 60 months, 72 months, 84 months, 96 months, 108 months, or 120 months.

43. A method of treating congenital sucrase-isomaltase deficiency (CSID) in a subject in need thereof, the method comprising administering to the subject an effective amount of the pharmaceutical composition of any one of claims 26-42.

44. The method of claim 43, wherein the pharmaceutical composition is administered to the subject together with a meal or snack.

45. A method of reducing sucrose and branched (1-6 linked) α-limit dextrin concentration in a subject, the method comprising administering to the subject an effective amount of the pharmaceutical composition of any one of claims 26-44.

46. The method of claim 45, wherein the method reduces sucrose and branched (1-6 linked) α-limit dextrin concentration in the subject as measured by a hydrogen breath test.

47. A method of treating congenital sucrase-isomaltase deficiency (CSID) in a subject in need thereof, the method comprising administering to the subject (i) an isomaltase enzyme of any one of claims 1-20 and (ii) a separate invertase enzyme.

48. The method of claim 47, wherein the isomaltase is the recombinant mutant L. fermentum isomaltase enzyme of any one of claims 1-20.

49. The method of claim 48, wherein the invertase is the recombinant mutant S. cerevisiae or invertase enzyme of any one of claims 35-38.

50. The method of any one of claims 43-49, wherein the subject is a mammal.

51. The method of any one of claims 43-49, wherein the subject is a human.
